SEQUENCE LISTING (81 sequences)

SEQ ID NO: 1 - protein, 450 aa, synthetic construct
  REGION 1..450: note = Synthesized_1901Ab1 Heavy Chain
EVQLLESGGGLVQPGGSLRLSCAASGFTFSSFAMTWVRQAPGKGLEWVSSLSGSGRSTYYAASMKGRFTISRDNSKNTLYLQMNSLRAEDTAIYYCAKSLSYYHYYFDYWGQGTLVTVTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Synthesized_1901Ab1 and 1901Ab2 Light Chain
DIQLTQSPSFLSASVGDRVPITCRASQGISNFLAWYQQKPGKAPELLIHSASTLQSGVPSRFSGSGSGTEFTLTISNLQPQDFATYYCQQVNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 717 aa, synthetic construct
  REGION 1..717: note = Synthesized_1901Ab2 Heavy Chain
EVQLLESGGGLVQPGGSLRLSCAASGFTFSSFAMTWVRQAPGKGLEWVSSLSGSGRSTYYAASMKGRFTISRDNSKNTLYLQMNSLRAEDTAIYYCAKSLSYYHYYFDYWGQGTLVTVTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 4 - protein, 450 aa, synthetic construct
  REGION 1..450: note = Synthesized_1901Ab3 Heavy Chain
EVQLLESGGGLVQPGGSLRLSCAASGFTFSSFAMTWVRQAPGKGLEWVSSLSGSGRSTYYAASMKGRFTISRDNSKNTLYLQMNSLRAEDTAIYYCAKSLSYYHYYFDYWGQGTLVTVTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 483 aa, synthetic construct
  REGION 1..483: note = Synthesized_1901Ab3 Light Chain
DIQLTQSPSFLSASVGDRVPITCRASQGISNFLAWYQQKPGKAPELLIHSASTLQSGVPSRFSGSGSGTEFTLTISNLQPQDFATYYCQQVNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 6 - protein, 454 aa, synthetic construct
  REGION 1..454: note = Synthesized_1923Ab4 Heavy Chain
QVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 213 aa, synthetic construct
  REGION 1..213: note = Synthesized_1923Ab4 Light Chain
SYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 451 aa, synthetic construct
  REGION 1..451: note = Synthesized_1912Ab1 Heavy Chain
QVQLVQSGAEVKKPGASVKVSCKASGNTFTSYDINWVRQATGQGLEWMGWMNPNSGNTGYAQKFQGRVTMTRDTSISTAYMELSSLRSEDTAVYYCVYYNYVWGSFRFDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
  REGION 1..214: note = Synthesized_1912 Ab1 and 1912 Ab3 Light Chain
DIQLTQSPSFLSASVGDRVTITCRASQGISSYLAWYQQKPGKAPKLLIYAASTLQSGVPSRFSGSGSGTEFTLAVSSLQPEDFATYYCQQLNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, synthetic construct
  REGION 1..448: note = Synthesized_1912Ab2 Heavy Chain
QVQLVESGGGVVQPGRSLRLSCTASGFTFSHYGMHWVRQAPGKGLEWVSFLWFDGSKDNYADSVKGRFTISRDNSKNMLYLRMDSLRAEDTAVYYCARNGGLTIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
  REGION 1..214: note = Synthesized_1912 Ab2 and 1912Ab4 Light Chain
AIQLTQSPSSLSASVGDRVTITCRASQGINSWLAWYQQKPEKAPKSLIYAASSLQSGVPSRFSGSGSGTDFTLTISSLQPEDFATYYCQQYNFYPYTFGQGTKLEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 718 aa, synthetic construct
  REGION 1..718: note = Synthesized_1912Ab3 Heavy Chain
QVQLVQSGAEVKKPGASVKVSCKASGNTFTSYDINWVRQATGQGLEWMGWMNPNSGNTGYAQKFQGRVTMTRDTSISTAYMELSSLRSEDTAVYYCVYYNYVWGSFRFDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 13 - protein, 715 aa, synthetic construct
  REGION 1..715: note = Synthesized_1912Ab4 Heavy Chain
QVQLVESGGGVVQPGRSLRLSCTASGFTFSHYGMHWVRQAPGKGLEWVSFLWFDGSKDNYADSVKGRFTISRDNSKNMLYLRMDSLRAEDTAVYYCARNGGLTIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 14 - protein, 713 aa, synthetic construct
  REGION 1..713: note = Synthesized_1925Ab1 Heavy Chain
EVQLVQSGAEVKKPGASVKVSCKASGYTFTTYYIHWVRQAPGQRLEWIGWIYPGNVNTKYTENFKDRATLTADKSASTAYMELSSLRSEDTAVYYCARGLYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 15 - protein, 214 aa, synthetic construct
  REGION 1..214: note = Synthesized_1925Ab1 and 1925Ab3 Light Chain
DIQMTQSPSSLSASVGDRVTITCKASQSVNNDVAWYQQKPGKAPKLLIYYASNRDTGVPSRFSGSGSGTDFTLTISSLQPEDFATYYCQQDYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 446 aa, synthetic construct
  REGION 1..446: note = Synthesized_1925Ab2 Heavy Chain
EVQLVQSGAEVKKPGASVKVSCKASGYTFTTYYIHWVRQAPGQRLEWIGWIYPGNVNTKYTENFKDRATLTADKSASTAYMELSSLRSEDTAVYYCARGLYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 483 aa, synthetic construct
  REGION 1..483: note = Synthesized_1925Ab2 Light Chain
DIQMTQSPSSLSASVGDRVTITCKASQSVNNDVAWYQQKPGKAPKLLIYYASNRDTGVPSRFSGSGSGTDFTLTISSLQPEDFATYYCQQDYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 18 - protein, 720 aa, synthetic construct
  REGION 1..720: note = Synthesized_1925Ab3 Heavy Chain
QVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVLGGGGSGGGGSGGGGSGGGGSEVQLVQSGAEVKKPGASVKVSCKASGYTFTTYYIHWVRQAPGQRLEWIGWIYPGNVNTKYTENFKDRATLTADKSASTAYMELSSLRSEDTAVYYCARGLYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 446 aa, synthetic construct
  REGION 1..446: note = Synthesized_1925Ab4 Heavy Chain
QVQLQQSGPELVKPGASVRISCKASGYTFTTYYIHWVKQRPGQGLEWIGWIYPGNVNTKYTENFKDKATLTADKSSSTAYMQLSSLTSEDSAVYFCARGLYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
  REGION 1..214: note = Synthesized_1925Ab4 Light Chain
SIVMTQTPKFLLVSAGDRVTITCKASQSVNNDVAWYQQKPGQSPKLLIYYASNRDTGVPDRFTGSGFGTDFTFTISSVQAEDLAVYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 120 aa, synthetic construct
  REGION 1..120: note = Synthesized_1901Ab1_VH
EVQLLESGGGLVQPGGSLRLSCAASGFTFSSFAMTWVRQAPGKGLEWVSSLSGSGRSTYYAASMKGRFTISRDNSKNTLYLQMNSLRAEDTAIYYCAKSLSYYHYYFDYWGQGTLVTVTS

SEQ ID NO: 22 - protein, 107 aa, synthetic construct
  REGION 1..107: note = Synthesized_1901Ab1 and 1901Ab2_VL
DIQLTQSPSFLSASVGDRVPITCRASQGISNFLAWYQQKPGKAPELLIHSASTLQSGVPSRFSGSGSGTEFTLTISNLQPQDFATYYCQQVNSYPLTFGGGTKVEIK

SEQ ID NO: 23 - protein, 127 aa, synthetic construct
  REGION 1..127: note = Synthesized_1923Ab4_VH
QVQLVESGGGVVQPGRSLRLSCAASGFTFSNYGMHWVRQAPGKGLEWVAIIWYDGINKYYADSVKGRFTISRDNSKNTLYLQMNSLKAEDTAVYYCARDQDYDILTGYYKEDYFDFWGQGTLVTVSS

SEQ ID NO: 24 - protein, 107 aa, synthetic construct
  REGION 1..107: note = Synthesized_1923Ab4_VL
SYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGGGTKLTVL

SEQ ID NO: 25 - protein, 121 aa, synthetic construct
  REGION 1..121: note = Synthesized_1912Ab1_VH
QVQLVQSGAEVKKPGASVKVSCKASGNTFTSYDINWVRQATGQGLEWMGWMNPNSGNTGYAQKFQGRVTMTRDTSISTAYMELSSLRSEDTAVYYCVYYNYVWGSFRFDDWGQGTLVTVSS

SEQ ID NO: 26 - protein, 107 aa, synthetic construct
  REGION 1..107: note = Synthesized_1912Ab1_VL
DIQLTQSPSFLSASVGDRVTITCRASQGISSYLAWYQQKPGKAPKLLIYAASTLQSGVPSRFSGSGSGTEFTLAVSSLQPEDFATYYCQQLNSYPYTFGQGTKLEIK

SEQ ID NO: 27 - protein, 118 aa, synthetic construct
  REGION 1..118: note = Synthesized_1912Ab2_VH
QVQLVESGGGVVQPGRSLRLSCTASGFTFSHYGMHWVRQAPGKGLEWVSFLWFDGSKDNYADSVKGRFTISRDNSKNMLYLRMDSLRAEDTAVYYCARNGGLTIFDYWGQGTLVTVSS

SEQ ID NO: 28 - protein, 107 aa, synthetic construct
  REGION 1..107: note = Synthesized_1912Ab2_VL
AIQLTQSPSSLSASVGDRVTITCRASQGINSWLAWYQQKPEKAPKSLIYAASSLQSGVPSRFSGSGSGTDFTLTISSLQPEDFATYYCQQYNFYPYTFGQGTKLEII

SEQ ID NO: 29 - protein, 116 aa, synthetic construct
  REGION 1..116: note = Synthesized_1925Ab4_VH(Hz)
EVQLVQSGAEVKKPGASVKVSCKASGYTFTTYYIHWVRQAPGQRLEWIGWIYPGNVNTKYTENFKDRATLTADKSASTAYMELSSLRSEDTAVYYCARGLYYFDFWGQGTTLTVSS

SEQ ID NO: 30 - protein, 107 aa, synthetic construct
  REGION 1..107: note = Synthesized_1925Ab4_VL(Hz)
DIQMTQSPSSLSASVGDRVTITCKASQSVNNDVAWYQQKPGKAPKLLIYYASNRDTGVPSRFSGSGSGTDFTLTISSLQPEDFATYYCQQDYSSPYTFGQGTKLEIK

SEQ ID NO: 31 - protein, 116 aa, synthetic construct
  REGION 1..116: note = Synthesized_1925Ab4_VH
QVQLQQSGPELVKPGASVRISCKASGYTFTTYYIHWVKQRPGQGLEWIGWIYPGNVNTKYTENFKDKATLTADKSSSTAYMQLSSLTSEDSAVYFCARGLYYFDFWGQGTTLTVSS

SEQ ID NO: 32 - protein, 107 aa, synthetic construct
  REGION 1..107: note = Synthesized_1925Ab4_VL
SIVMTQTPKFLLVSAGDRVTITCKASQSVNNDVAWYQQKPGQSPKLLIYYASNRDTGVPDRFTGSGFGTDFTFTISSVQAEDLAVYFCQQDYSSPYTFGGGTKLEIK

SEQ ID NO: 33 - protein, 5 aa, synthetic construct
  REGION 1..5: note = Synthesized_CDR1
SFAMT

SEQ ID NO: 34 - protein, 17 aa, synthetic construct
  REGION 1..17: note = Synthesized_CDR2
SLSGSGRSTYYAASMKG

SEQ ID NO: 35 - protein, 11 aa, synthetic construct
  REGION 1..11: note = Synthesized_CDR3
SLSYYHYYFDY

SEQ ID NO: 36 - protein, 11 aa, synthetic construct
  REGION 1..11: note = Synthesized_CDR1
RASQGISNFLA

SEQ ID NO: 37 - protein, 7 aa, synthetic construct
  REGION 1..7: note = Synthesized_CDR2
SASTLQS

SEQ ID NO: 38 - protein, 9 aa, synthetic construct
  REGION 1..9: note = Synthesized_CDR3
QQVNSYPLT

SEQ ID NO: 39 - protein, 5 aa, synthetic construct
  REGION 1..5: note = Synthesized_CDR1
NYGMH

SEQ ID NO: 40 - protein, 17 aa, synthetic construct
  REGION 1..17: note = Synthesized_CDR2
IIWYDGINKYYADSVKG

SEQ ID NO: 41 - protein, 18 aa, synthetic construct
  REGION 1..18: note = Synthesized_CDR3
DQDYDILTGYYKEDYFDF

SEQ ID NO: 42 - protein, 11 aa, synthetic construct
  REGION 1..11: note = Synthesized_CDR1
SGDALPKKYAY

SEQ ID NO: 43 - protein, 7 aa, synthetic construct
  REGION 1..7: note = Synthesized_CDR2
EDSKRPS

SEQ ID NO: 44 - protein, 10 aa, synthetic construct
  REGION 1..10: note = Synthesized_CDR3
YSTDSSGNRV

SEQ ID NO: 45 - protein, 5 aa, synthetic construct
  REGION 1..5: note = Synthesized_HCDR1
SYDIN

SEQ ID NO: 46 - protein, 17 aa, synthetic construct
  REGION 1..17: note = Synthesized_HCDR2
WMNPNSGNTGYAQKFQG

SEQ ID NO: 47 - protein, 12 aa, synthetic construct
  REGION 1..12: note = Synthesized_HCDR3
YNYVWGSFRFDD

SEQ ID NO: 48 - protein, 11 aa, synthetic construct
  REGION 1..11: note = Synthesized_LCDR1
RASQGISSYLA

SEQ ID NO: 49 - protein, 7 aa, synthetic construct
  REGION 1..7: note = Synthesized_LCDR2
AASTLQS

SEQ ID NO: 50 - protein, 9 aa, synthetic construct
  REGION 1..9: note = Synthesized_LCDR3
QQLNSYPYT

SEQ ID NO: 51 - protein, 5 aa, synthetic construct
  REGION 1..5: note = Synthesized_HCDR1
HYGMH

SEQ ID NO: 52 - protein, 17 aa, synthetic construct
  REGION 1..17: note = Synthesized_HCDR2
FLWFDGSKDNYADSVKG

SEQ ID NO: 53 - protein, 9 aa, synthetic construct
  REGION 1..9: note = Synthesized_HCDR3
NGGLTIFDY

SEQ ID NO: 54 - protein, 11 aa, synthetic construct
  REGION 1..11: note = Synthesized_LCDR1
RASQGINSWLA

SEQ ID NO: 55 - protein, 7 aa, synthetic construct
  REGION 1..7: note = Synthesized_LCDR2
AASSLQS

SEQ ID NO: 56 - protein, 9 aa, synthetic construct
  REGION 1..9: note = Synthesized_LCDR3
QQYNFYPYT

SEQ ID NO: 57 - protein, 5 aa, synthetic construct
  REGION 1..5: note = Synthesized_HCDR1
TYYIH

SEQ ID NO: 58 - protein, 17 aa, synthetic construct
  REGION 1..17: note = Synthesized_HCDR2
WIYPGNVNTKYTENFKD

SEQ ID NO: 59 - protein, 7 aa, synthetic construct
  REGION 1..7: note = Synthesized_HCDR3
GLYYFDF

SEQ ID NO: 60 - protein, 11 aa, synthetic construct
  REGION 1..11: note = Synthesized_LCDR1
KASQSVNNDVA

SEQ ID NO: 61 - protein, 7 aa, synthetic construct
  REGION 1..7: note = Synthesized_LCDR2
YASNRDT

SEQ ID NO: 62 - protein, 9 aa, synthetic construct
  REGION 1..9: note = Synthesized_LCDR3
QQDYSSPYT

SEQ ID NO: 63 - protein, 254 aa, synthetic construct
  REGION 1..254: note = Synthesized_Anti-CD137 scFv* with 4x (G4S) linker
QVQLVESGGGVVQPGRSLRLSCAASGFTFSNYGMHWVRQAPGKCLEWVAIIWYDGINKYYADSVKGRFTISRDNSKNTLYLQMNSLKAEDTAVYYCARDQDYDILTGYYKEDYFDFWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNRVFGCGTKLTVL

SEQ ID NO: 64 - protein, 15 aa, synthetic construct
  REGION 1..15: note = Synthesized_3x (G4S) linker
GGGGSGGGGSGGGGS

SEQ ID NO: 65 - protein, 20 aa, synthetic construct
  REGION 1..20: note = Synthesized_4x (G4S) linker
GGGGSGGGGSGGGGSGGGGS

SEQ ID NO: 66 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 330 aa, Homo sapiens [sequence omitted]

SEQ ID NO: 68 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 69 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 107 aa, Homo sapiens [sequence omitted]

SEQ ID NO: 71 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - protein, 261 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 73 - protein, 261 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKTYDGGAHTEDELQSYPSKHDYV

SEQ ID NO: 74 - protein, 261 aa, synthetic construct
MSVTACQGLGFVVSLIGFAGIIAATCMDQWSTQDLYNNPVTAVFNYQGLWRSCVRESSGFTECRGYFTLLGLPAMLQAVRALMIVGIVLGVIGILVSIFALKCIRIGSMDDSAKAKMTLTSGIMFIISGVCAIIGVSVFANMLVTNFWMSTANMYSGMGGMVQTVQTRYTFGAALFVGWIAGGLTLIGGVMMCIACRGLTPDDRNFKAVSYHASGQNVAYKPGGFKASTGFGSNARNKKIYDGGARTEDDEQSHPTKYDYV

SEQ ID NO: 75 - protein, 220 aa, synthetic construct
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 76 - protein, 220 aa, synthetic construct
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSRGPSHYMARYSTSAPAISRGPSEYPTKNYV

SEQ ID NO: 77 - protein, 219 aa, synthetic construct
MASTGLQILGIVLTLLGWVNALVSCALPMWKVTAFIGNSIVVAQMVWEGLWMSCVVQSTGQMQCKVYDSLLALPQDLQAARALCVVTLLIVLLGLLVYLAGAKCTTCVEDRNSKSRLVLISGIIFVISGVLTLIPVCWTAHSIIQDFYNPLVADAQKRELGASLYLGWAASGLLLLGGGLLCCACSSGGTQGPRHYMACYSTSVPHSRGPSEYPTKNYV

SEQ ID NO: 78 - protein, 510 aa, synthetic construct
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 79 - protein, 510 aa, synthetic construct
MPLSLGAEMWGPEAWLLLLLLLASFTGRCPAGELETSDVVTVVLGQDAKLPCFYRGDSGEQVGQVAWARADAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHVGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRTEEEEDQDEGIKQAMNHFVQENGTLRAKPTGNGIYINGRGHLV

SEQ ID NO: 80 - protein, 508 aa, synthetic construct
MPLSLGAEMWGPEAWLLLLFLASFTGRYSAGELETSDLVTVVLGQDAKLPCFYRGDPDEQVGQVAWARVDPNEGTRELALLHSKYGLHVSPAYEDRVEQPPPPRDPLDGSILLRNAVQADEGEYECRVSTFPAGSFQARMRLRVLVPPLPSLNPGPPLEEGQGLTLAASCTAEGSPAPSVTWDTEVKGTQSSRSFKHSRSAAVTSEFHLVPSRSMNGQPLTCVVSHPGLLQDQRITHTLQVAFLAEASVRGLEDQNLWHVGREGATLKCLSEGQPPPKYNWTRLDGPLPSGVRVKGDTLGFPPLTTEHSGVYVCHVSNELSSRASQVTVEVLDPEDPGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRTEEEDDQDEGIKQAMNHFVQENGTLRAKPTGNGIYINGRGHLV

SEQ ID NO: 81 - protein, 508 aa, synthetic construct
MPLSLGAEMWGPEAWLRLLFLASFTGQYSAGELETSDVVTVVLGQDAKLPCFYRGDPDEQVGQVAWARVDPNEGIRELALLHSKYGLHVNPAYEDRVEQPPPPRDPLDGSVLLRNAVQADEGEYECRVSTFPAGSFQARMRLRVLVPPLPSLNPGPPLEEGQGLTLAASCTAEGSPAPSVTWDTEVKGTQSSRSFTHPRSAAVTSEFHLVPSRSMNGQPLTCVVSHPGLLQDRRITHTLQVAFLAEASVRGLEDQNLWQVGREGATLKCLSEGQPPPKYNWTRLDGPLPSGVRVKGDTLGFPPLTTEHSGVYVCHVSNELSSRDSQVTVEVLDPEDPGKQVDLVSASVIIVGVIAALLFCLLVVVVVLMSRYHRRKAQQMTQKYEEELTLTRENSIRRLHSHHSDPRSQPEESVGLRAEGHPDSLKDNSSCSVMSEEPEGRSYSTLTTVREIETQTELLSPGSGRTEEDDDQDEGIKQAMNHFVQENGTLRAKPTGNGIYINGRGHLV

(19) United States
(12) Patent Application Publication
(10) Pub. No.: US 2024/0383978 A1
(43) Pub. Date: Nov. 21, 2024

(54) BISPECIFIC BINDING PROTEINS THAT BIND CD137 AND A TUMOR ASSOCIATED ANTIGEN

(71) Applicant: NOVAROCK BIOTHERAPEUTICS, LTD., Ewing, NJ (US)

(72) Inventors: Yi Pei, Paoli, PA (US); Ming Lei, Princeton, NJ (US); Haichun Huang, Fremont, CA (US); Yick Loi, East Brunswick, NJ (US); Chang Hung Chen, Germantown, MD (US); Han LI, Newtown, PA (US)

(21) Appl. No.: 18/688,630

(22) PCT Filed: Sep. 1, 2022
(86) PCT No.: PCT/US2022/075846

§ 371 Date: Mar. 1, 2024

Related U.S. Application Data
(60) Provisional application No. 63/327,700, filed on Apr. 5, 2022.
(60) Provisional application No. 63/240,402, filed on Sep. 3, 2021.

(51) Int. Cl. C07K 16/28; A61K 39/00; A61P 35/00; C07K 16/30

(52) U.S. Cl. CPC C07K 16/28; A61P 35/00; C07K 16/2878; C07K 16/30; A61K 2039/505; C07K 2317/31; C07K 2317/622; C07K 2317/73

(57) ABSTRACT

The present disclosure provides bispecific binding proteins and fragments thereof which bind to human CD137 and a tumor associated antigen (e.g., Claudin-6, Claudin 18.2, or Nectin-4), to polynucleotide sequences encoding these antibodies and to cells producing them. The disclosure further relates to therapeutic compositions comprising these antibodies, and to methods of their use for cancer detection, prognosis and antibody-based immunotherapy.

### CROSS REFERENCE TO RELATED APPLICATIONS

This international patent application claims the benefit of U.S. Provisional Patent Application No. 63/240,402, filed on Sep. 3, 2021, and U.S. Provisional Patent Application No. 63/327,700, filed on Apr. 5, 2022, the entire contents of which are incorporated by reference herein.

### STATEMENT REGARDING SEQUENCE LISTING

The Sequence Listing associated with this application is provided in XML format in lieu of a paper copy, and is hereby incorporated by reference into the specification. The name of the XLM file containing the Sequence Listing is 122863-5007_Sequence_Listing_ST.26.xml. The text file is about 111,345 bytes, was created on or about Aug. 28, 2022, and is being submitted electronically via EFS-Web.

### FIELD

The present disclosure is in the field of immunotherapy and relates to bispecific binding proteins and fragments thereof which bind to human CD137 and a tumor associated antigen (e.g., Claudin-6, Claudin 18.2, or Nectin-4), to polynucleotide sequences encoding these antibodies and to cells producing them. The disclosure further relates to therapeutic compositions comprising these bispecific binding proteins, and to methods of their use for cancer detection, prognosis and antibody-based immunotherapy.

### BACKGROUND

Activation of T cells plays a central role in antitumor immunity. Two key signals are required to activate naïve T cells. Signal one is provided through the T-cell receptor (TCR), while signal two is that of co-stimulation. The CD28: B7 molecules are some of the best-studied costimulatory pathways, thought to be the main mechanism through which primary T cell stimulation occurs. However, a number of other molecules have been identified which serve to amplify and diversify the T cell response following initial T cell activation. These include CD137/CD137 ligand (CD137L) molecules, also known as 4-1BB: 4-1BB ligand (4-1BBL).

CD137 (4-1BB, tumor necrosis factor receptor superfamily 9) is a member of the TNF-receptor superfamily (TNFRSF) and is a costimulatory molecule expressed following the activation of immune cells, both innate and adaptive immune cells. CD137 plays an essential role in modulating the activity of various immune cells. Therapies targeting the CD137/CD137L signaling pathway have been shown to have antitumor effects in a number of model systems, and agonistic anti-CD137 antibodies have also entered clinical development (Yonezawa et al., Clin. Cancer Res. 2015 Jul. 15; 21 (14): 3113-20; Tolcher et al., Clin Cancer Res. 2017 Sep. 15; 23 (18): 5349-5357). CD137 agonists enhance immune cell proliferation, survival, secretion of cytokines and cytolytic activity CD8 T cells. Many other studies showed that activation of CD137 enhances immune response to eliminate tumors in mice. In the clinic, CD137 monoclonal antibody therapies have shown promising anti-tumor effects, but systemic immune stimulation has induced dose-limiting hepatic toxicities (Chester, C. et al., Cancer Immunol Immunother 65, 1243-1248 (2016); Segal, N. H et al., Clin. Cancer Res. 2017, 23, 1929-1936).

New CD137 agonist moieties are being developed, aiming at potent co-stimulation targeted to the tumor microenvironment (TME) to avoid side effects of liver inflammation and broaden the therapeutic window. Different approaches are applied. The most advanced ones under clinical development are the CD137-based bispecific constructs designed to bring CD137 co-stimulation specifically to the TME, such as bispecific Ab targeting a tumor antigen (e.g., a TAA or a TSA) and CD137. Anti-tumor activities are possibly achieved by directing the host immune system toward tumor-associated antigens. Linking tumor cells with CD137 expressing T cells to increase cellular cytotoxicity represents a promising strategy in cancer therapy.

A CD137-Her2 bispecific antibody indicated good tolerability of the construct and showed evidence of clinical activity (Hinner M J et al., Clin Cancer Res. 2019 Oct. 1; 25 (19): 5878-5889; Piha-Paul S et al., Phase 1 dose escalation study of PRS-343, a HER2/4-1BB bispecific molecule, in patients with HER2+ malignancies. 34th Annual Meeting & Pre-Conference Programs of the Society for Immunotherapy of Cancer, National Harbor, MD, 2019), bispecific Ab targeting the tumor antigen 5T4 and CD137 also demonstrated good pre-clinical activity (Nelson M et al., Potent tumor-directed T cell activation and tumor inhibition induced by ALG.APV-527, a 4-1BB×5T4 ADAPTIR™ bispecific antibody, 34th Annual Meeting & Pre-Conference Programs of the Society for Immunotherapy of Cancer, National Harbor, MD, 2019).

Claudin superfamily members are key components of tight junction, maintaining cellular polarity and sealing the spaces between adjacent cells. Claudin 6 (CLDN6) is a carcinoembryonic protein expressed during early development but silenced in healthy adult human tissues. CLDN6 expression has been reported in a wide range of non-hematological cancer such as pediatric brain tumors, gastric adenocarcinomas and germ cell tumors, and ovarian and testicle cancers. Its expression is often correlated with a poor prognosis. Claudin18.2 is widely expressed in a wide range of human malignancies including gastric, esophageal, pancreatic, lung, and ovarian cancers. Claudin18.2 has a superior target safety profile. In normal tissue, Claudin18.2 expression is restricted to the stomach and only on short-lived differentiated cells. Nectin family proteins mediate cell-cell adhesion through homophilic and heterophilic trans-interactions, in which heterophilic trans-interactions are much stronger than homophilic trans-interaction. As a Nectin protein family member, nectin-4 is an important driver for tumorigenesis and metastasis. Over-expression of Nectin-4 in cancer tissue is associated with cancer progression and poor prognosis.

Cancers of epithelial origin represent a significant global medical challenge that impacts patients, their families, and the health care system. For the unmet medical needs of patients with tumors overexpress epithelial tumor antigens, such as CLDN6, CLDN18.2, or Nectin-4, specific binding proteins that bind CD137 and these tumor antigens that alone, or in combination with other agents can be used for antibody-based immunotherapy, providing potentially effective and safe treatment solutions.

### SUMMARY

The present disclosure addresses the above need by providing bispecific binding proteins that bind to CD137 and a tumor associated antigen (TAA). In particular embodiments the disclosure provides bispecific antibodies that bind to the tumor specific antigens CLDN18.2, CLDN6 or Nectin-4, and to the costimulatory CD137 receptor. Such bispecific binding proteins may be useful for the treatment of a disease or disorder such as cancer.

Also provided herein is a bispecific binding protein that binds a tumor associated antigen and CD137 comprising: (a) an antibody scaffold module comprising a first antigen-binding site that binds the tumor associated antigen and a second antigen-binding site that binds the tumor associated antigen; and (b) at least one first binding module comprising a third antigen-binding site that binds CD137.

In some embodiments, the tumor associated antigen is selected from the group consisting of: Claudin 6, Claudin 18.2, and Nectin-4.

In some embodiments, the tumor associated antigen is Claudin 6.

In some embodiments, the tumor associated antigen is Claudin 18.2.

In some embodiments, the tumor associated antigen is Nectin-4.

In some embodiments, the antibody scaffold module is an IgG.

In some embodiments, the first antigen-binding site and the second antigen-binding site both bind Claudin 6 and comprise: (i) a heavy chain variable region sequence comprising CDR1: SEQ ID NO: 45, CDR2: SEQ ID NO: 46, and CDR3: SEQ ID NO: 47; and a light chain variable region sequence comprising CDR1: SEQ ID NO: 48, CDR2: SEQ ID NO: 49, and CDR3: SEQ ID NO: 50; or (ii) a heavy chain variable region sequence comprising CDR1: SEQ ID NO: 51, CDR2: SEQ ID NO: 52, and CDR3: SEQ ID NO: 53; and a light chain variable region sequence comprising CDR1: SEQ ID NO: 54, CDR2: SEQ ID NO: 55, and CDR3: SEQ ID NO: 56.

In some embodiments, the first antigen-binding site and the second antigen-binding site both bind Claudin 6 and comprise a heavy chain variable region sequence as set forth in SEQ ID NO: 25 or SEQ ID NO: 27; and a light chain variable region sequence as set forth in SEQ ID NO: 26 or SEQ ID NO: 28.

In some embodiments, the first antigen-binding site and the second antigen-binding site both bind Claudin 6 and comprise: (i) a heavy chain variable region sequence as set forth in SEQ ID NO: 25 and a light chain variable region sequence as set forth in SEQ ID NO: 26; or (ii) a heavy chain variable region sequence as set forth in SEQ ID NO: 27 and a light chain variable region sequence as set forth in SEQ ID NO: 28.

In some embodiments, the first antigen-binding site and the second antigen-binding site both bind Claudin 18.2 and comprise: a heavy chain variable region sequence comprising CDR1: SEQ ID NO: 33, CDR2: SEQ ID NO: 34, and CDR3: SEQ ID NO: 35; and a light chain variable region sequence comprising CDR1: SEQ ID NO: 36, CDR2: SEQ ID NO: 37, and CDR3: SEQ ID NO: 38.

In some embodiments, the first antigen-binding site and the second antigen-binding site both bind Claudin 18.2 and comprise a heavy chain variable region sequence as set forth in SEQ ID NO: 21; and a light chain variable region sequence as set forth in SEQ ID NO: 22.

In some embodiments, the first antigen-binding site and the second antigen-binding site both bind Nectin-4 and comprise: a heavy chain variable region sequence comprising CDR1: SEQ ID NO: 57, CDR2: SEQ ID NO: 58, and CDR3: SEQ ID NO: 59; and a light chain variable region sequence comprising CDR1: SEQ ID NO: 60, CDR2: SEQ ID NO: 61, and CDR3: SEQ ID NO: 62.

In some embodiments, the first antigen-binding site and the second antigen-binding site both bind Nectin-4 and comprise a heavy chain variable region sequence as set forth in SEQ ID NO: 29 or SEQ ID NO: 31; and a light chain variable region sequence as set forth in SEQ ID NO: 30 or SEQ ID NO: 32.

In some embodiments, the first antigen-binding site and the second antigen-binding site both bind Nectin-4 and comprise: (i) a heavy chain variable region sequence as set forth in SEQ ID NO: 29 and a light chain variable region sequence as set forth in SEQ ID NO: 30; or (ii) a heavy chain variable region sequence as set forth in SEQ ID NO: 31 and a light chain variable region sequence as set forth in SEQ ID NO: 32.

In some embodiments, the bispecific binding protein comprises one first binding module.

In some embodiments, the binding protein comprises two first binding modules.

In some embodiments, the first binding module is an antibody fragment.

In some embodiments, the antibody fragment is an scFv.

In some embodiments, the first binding module binds CD137.

In some embodiments, the first binding module is an scFV that binds CD137.

In some embodiments, the antibody scaffold module comprises two heavy chain sequences both having a C-terminus and an N-terminus, and wherein the antibody scaffold module comprises two light chain sequences both having a C-terminus and a N-terminus. The first binding module is covalently attached to the C-terminus of one or both of the antibody scaffold module heavy chain sequences, the C-terminus of one or both of the antibody scaffold module light chain sequences, the N-terminus of one or both of the antibody scaffold module heavy chain sequences, the N-terminus of one or both of the antibody scaffold module light chain sequences, or combinations thereof, and wherein the first binding module and the antibody scaffold module are covalently attached to each other directly or through an interlinker.

In some embodiments, the first binding module and the antibody scaffold module are covalently attached to each other through an interlinker having a sequence as set forth in SEQ ID NO: 64 or SEQ ID NO: 65.

In some embodiments, the first binding module is covalently attached to the C-terminus of both of the antibody scaffold module heavy chain sequences.

In some embodiments, the first binding module is covalently attached to the C-terminus of both of the antibody scaffold module light chain sequences.

In some embodiments, the first binding module is covalently attached to the N-terminus of both of the antibody scaffold module heavy chain sequences.

In some embodiments, the first binding module binds CD137 and comprises: a heavy chain variable region sequence comprising CDR1: SEQ ID NO: 39, CDR2: SEQ ID NO: 40, and CDR3: SEQ ID NO: 41; and a light chain variable region sequence comprising CDR1: SEQ ID NO: 42, CDR2: SEQ ID NO: 43, and CDR3: SEQ ID NO: 44.

In some embodiments, the first binding module bind CD137 and comprises: a heavy chain variable region sequence as set forth in SEQ ID NO: 23; and a light chain variable region sequence as set forth in SEQ ID NO: 24.

In some embodiments, the bispecific binding protein comprises two first binding modules that bind CD137 and wherein: the first antigen-binding site and the second antigen-binding site both bind Claudin 18.2 and comprise a heavy chain variable region sequence as set forth in SEQ ID NO: 21, and a light chain variable region sequence as set forth in SEQ ID NO: 22; the first binding modules each comprise a heavy chain variable region sequence as set forth in SEQ ID NO: 23, and a light chain variable region sequence as set forth in SEQ ID NO: 24, wherein the first binding modules are separately attached to the C-terminus of each heavy chain in the antibody scaffold module by a glycine-serine linker.

In some embodiments, the glycine-serine linker is a 3×G4S linker (SEQ ID NO: 64).

In some embodiments, the heavy chain variable region sequence and the light chain variable region sequence in the first binding modules are attached by a glycine-serine linker.

In some embodiments, the glycine-serine linker is a 4×G4S linker (SEQ ID NO: 65).

In some embodiments, the heavy chain of the antibody scaffold module, the glycine-serine linker, and the first binding module comprise a sequence as set forth in SEQ ID NO: 3.

In some embodiments, the bispecific binding protein comprises two first binding modules that bind CD137 and wherein: the first antigen-binding site and the second antigen-binding site both bind Claudin 18.2 and comprise a heavy chain variable region sequence as set forth in SEQ ID NO: 21, and a light chain variable region sequence as set forth in SEQ ID NO: 22; the first binding modules each comprise a heavy chain variable region sequence as set forth in SEQ ID NO: 23, and a light chain variable region sequence as set forth in SEQ ID NO: 24, wherein the first binding modules are separately attached to the C-terminus of each light chain in the antibody scaffold module by a glycine-serine linker.

In some embodiments, the glycine-serine linker is a 3×G4S linker (SEQ ID NO: 64).

In some embodiments, the heavy chain variable region sequence and the light chain variable region sequence in the first binding modules are attached by a glycine-serine linker.

In some embodiments, the glycine-serine linker is a 4×G4S linker (SEQ ID NO: 65).

In some embodiments, the light chain of the antibody scaffold module, the glycine-serine linker, and the first binding module comprise a sequence as set forth in SEQ ID NO: 5.

In some embodiments, the bispecific binding protein comprises two first binding modules that bind CD137 and wherein: the first antigen-binding site and the second antigen-binding site both bind Claudin 6 and comprise a heavy chain variable region sequence as set forth in SEQ ID NOs: 25 or 27, and a light chain variable region sequence as set forth in SEQ ID NOs: 26 or 28; the first binding modules each comprise a heavy chain variable region sequence as set forth in SEQ ID NO: 23, and a light chain variable region sequence as set forth in SEQ ID NO: 24, wherein the first binding modules are separately attached to the C-terminus of each heavy chain in the antibody scaffold module by a glycine-serine linker.

In some embodiments, the glycine-serine linker is a 3×G4S linker (SEQ ID NO: 64).

In some embodiments, the heavy chain variable region sequence and the heavy chain variable region sequence in the first binding modules are attached by a glycine-serine linker.

In some embodiments, the glycine-serine linker is a 3×G4S linker (SEQ ID NO: 64).

In some embodiments, the heavy chain of the antibody scaffold module, the glycine-serine linker, and the first binding module comprise a sequence as set forth in SEQ ID NO: 12, 13 or 72.

In some embodiments, the bispecific binding protein comprises two first binding modules that bind CD137 and wherein: the first antigen-binding site and the second antigen-binding site both bind Nectin-4 and comprise a heavy chain variable region sequence as set forth in SEQ ID NOs: 29 or 31, and a light chain variable region sequence as set forth in SEQ ID NOs: 30 or 32; the first binding modules each comprise a heavy chain variable region sequence as set forth in SEQ ID NO: 23, and a light chain variable region sequence as set forth in SEQ ID NO: 24, wherein the first binding modules are separately attached to the C-terminus of each light chain in the antibody scaffold module by a glycine-serine linker.

In some embodiments, the glycine-serine linker is a 3×G4S linker (SEQ ID NO: 64).

In some embodiments, the heavy chain variable region sequence and the light chain variable region sequence in the first binding modules are attached by a glycine-serine linker.

In some embodiments, the glycine-serine linker is a 4×G4S linker (SEQ ID NO: 65).

In some embodiments, the light chain of the antibody scaffold module, the glycine-serine linker, and the first binding module comprise a sequence as set forth in SEQ ID NO: 17.

In some embodiments, the bispecific binding protein comprises two first binding modules that bind CD137 and wherein: the first antigen-binding site and the second antigen-binding site both bind Nectin-4 and comprise a heavy chain variable region sequence as set forth in SEQ ID NOs: 29 or 31, and a light chain variable region sequence as set forth in SEQ ID NOs: 30 or 32; the first binding modules each comprise a heavy chain variable region sequence as set forth in SEQ ID NO: 23, and a light chain variable region sequence as set forth in SEQ ID NO: 24, wherein the first binding modules are separately attached to the N-terminus of each heavy chain in the antibody scaffold module by a glycine-serine linker.

In some embodiments, the glycine-serine linker is a 4×G4S linker (SEQ ID NO: 65).

In some embodiments, the heavy chain variable region sequence and the light chain variable region sequence in the first binding modules are attached by a glycine-serine linker.

In some embodiments, the glycine-serine linker is a 4×G4S linker (SEQ ID NO: 65).

In some embodiments, the first binding module, the glycine-serine linker, and the heavy chain of the antibody scaffold module comprise a sequence as set forth in SEQ ID NO: 18.

In some embodiments, the bispecific binding protein comprises two first binding modules that bind CD137 and wherein: the first antigen-binding site and the second antigen-binding site both bind Nectin-4 and comprise a heavy chain variable region sequence as set forth in SEQ ID NOs: 29 or 31, and a light chain variable region sequence as set forth in SEQ ID NOs: 30 or 32; the first binding modules each comprise a heavy chain variable region sequence as set forth in SEQ ID NO: 23, and a light chain variable region sequence as set forth in SEQ ID NO: 24, wherein the first binding modules are separately attached to the C-terminus of each heavy chain in the antibody scaffold module by a glycine-serine linker.

In some embodiments, the glycine-serine linker is a 3×G4S linker (SEQ ID NO: 64).

In some embodiments, the heavy chain variable region sequence and the light chain variable region sequence in the first binding modules are attached by a glycine-serine linker.

In some embodiments, the glycine-serine linker is a 4×G4S linker (SEQ ID NO: 65).

In some embodiments, the heavy chain of the antibody scaffold module, the glycine-serine linker, and the first binding module comprise a sequence as set forth in SEQ ID NO: 14.

In some embodiments, the antibody scaffold module further comprises a constant region.

In some embodiments, the constant region comprises one or more Fc silencing mutations.

In some embodiments, the Fc silencing mutation can be L234A/L235A (LALA) alone or in combination with P329A mutation (LALAP) or N297A

In some embodiments, the constant region comprises SEQ ID NO: 66, SEQ ID NO: 67, SEQ ID NO: 68, SEQ ID NO: 69, or SEQ ID NO: 73.

Also provided herein is a bispecific binding protein that binds a tumor associated antigen and CD137 comprising: (a) an antibody scaffold module comprising a means for binding the tumor associated antigen via a first antigen-binding site and a second antigen-binding site; and (b) at least one first binding module comprising a means for binding CD137 via a third antigen-binding site.

The present disclosure also provides a pharmaceutical composition comprising the bispecific binding protein as disclosed herein and a pharmaceutically acceptable carrier.

The present disclosure also provides a method of treating or preventing cancer, the method comprising administering the bispecific binding protein as disclosed herein to a patient in need thereof.

Also provided herein is an isolated polynucleotide comprising a sequence encoding the bispecific binding protein as disclosed herein. The present disclosure also provides vectors or cells comprising the polynucleotide disclosed herein. Also provided herein is a method for the production of the bispecific binding protein as disclosed herein comprising culturing the cell disclosed herein.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 FIGS. 1A-1C provide the amino acid sequences of the heavy chain and light chain sequences of the CLDN18.2/CD137, CLDN6/CD137, and Nectin-4/CD137 bispecifics.

FIG. 1D provides the amino acid sequences of the VH and VL domains of the binding proteins that bind CD137, CLDN6, CLDN18.2, or Nectin-4. Sequences of the other components of the TAA/CD137 bispecific proteins are also provided. The CDR sequences (Kabat numbering) of the VH and VL domains are underlined in their respective variable domain sequences. Sequence identifiers are provided.

FIG. 2 shows three exemplary bispecific binding protein formats, including i) a first having an antibody scaffold module with two antigen binding sites that bind a tumor associated antigen and two first binding modules (e.g., scFvs) that bind CD137 each separately attached to the C-terminus of the heavy chain constant regions of the antibody scaffold module (BsAb_A), ii) a second having an antibody scaffold module with two antigen binding sites that bind a tumor associated antigen and two first binding modules (e.g., scFvs) that binds CD137 each separately attached to the C-terminus of the light chain constant regions of the antibody scaffold module (BsAb_B), and iii) a third having an antibody scaffold module with two antigen binding sites that bind a tumor associated antigen and two first binding modules (e.g., scFvs) that binds CD137 each separately attached to the N-terminus of the heavy chain variable regions of the antibody scaffold module (BsAb_C).

FIG. 3 shows the heavy and light chain composition of several binding proteins, including 1901Ab1, 1901Ab2, 1901Ab3, 1923Ab4, 1912Ab1, 1912Ab2, 1912Ab3, 1912Ab4, 1912Ab5, 1925Ab1, 1925Ab2, 1925Ab3, and 1925Ab4.

FIG. 4 shows the composition of the antibody scaffold module and binding module (if present) of several binding proteins, including 1901Ab1, 1901Ab2, 1901Ab3, 1923Ab4, 1912Ab1, 1912Ab2, 1912Ab3, 1912Ab4, 1912Ab5, 1925Ab1, 1925Ab2, 1925Ab3, and 1925Ab4.

FIG. 5 shows the sequence identifiers for the sequences in several binding proteins, including 1901Ab1, 1901Ab2, 1901Ab3, 1923Ab4, 1912Ab1, 1912Ab2, 1912Ab3, 1912Ab4, 1912Ab5, 1925Ab1, 1925Ab2, 1925Ab3, and 1925Ab4. For 1901Ab2, 1912Ab3, 1912Ab4, 1912Ab5, 1925Ab1, and 1925Ab3, the heavy chain sequence includes the amino acid sequence of the heavy chain of the antibody scaffold module, the glycine-serine linker, and the first binding module. For 1901Ab3 and 1925Ab2, the light chain sequence includes the amino acid sequence of the light chain of the antibody scaffold module, the glycine-serine linker, and the first binding module.

FIGS. 6A-B shows the binding activity towards tumor antigen Claudin6. FIG. 6A shows monospecific antibodies (1912Ab1 and 1912Ab2) and bispecific antibodies CLDN6/CD137 (1912Ab3 and 1912Ab4) BsAbs to human Claudin 6 on the cell surface of NEC8 wild-type cells compared to NEC8 CLDN6 knock-out cells. FIG. 6B shows the bsAb 1912Ab5 binds to NEC8 wild-type cells. FIG. 6C shows that 1912Ab5 selectively binds to Claudin6, not Claudin9.

FIGS. 7A-B shows CD137 binding activity. FIG. 7A shows the surface plasmon resonance (SPR) binding analysis of 1912Ab5 binding to CD137. FIG. 7B shows human CD137 binding of 1912Ab3, 1912Ab4 and 1923Ab4 in a HEK-CD137 cell-based binding assay. FIG. 7C shows dose response binding curves of 1912Ab5 and Urelumab-NR binding to human CD137. Urelumab-NR is an in-house control anti-CD137 antibody based on the publicly available information published in the U.S. Pat. No. 7,288,638

FIGS. 8A-D shows Claudin-6 dependent activation of CD137 signaling by CLDN6/CD137 BsAbs using Jurkat T cell CD137 NFKB reporter cells. FIG. 8A shows the activity from 1912Ab3, 1912Ab4 or benchmark control Urelumab-NR in the co-culture assay in the presence of the NEC8 wild-type cells or Claudin6 knock-out NEC8 cells. FIG. 8B shows the dose-dependent activity CLDN6/CD137 BsAbs 1912Ab3, 1912Ab4 or benchmark control Urelumab-NR in the signaling assay in the presence of the NEC8 wild-type cells. FIG. 8C shows the NFKB activation by 1912Ab5 or Urelumab-NR in the co-culture signaling assay using NEC8 target cells. FIG. 8D shows the NFKB activation by 1912Ab5 or Urelumab-NR in the co-culture signaling assay using OV90 target cells.

FIG. 9 shows Claudin 6 dependent activation of CD8 T cells inducing IFNγ secretion by CLDN6/CD137 BsAbs. FIG. 9A shows the activity from 1912Ab3, 1912Ab4 or benchmark control Urelumab-NR in the co-culture assay in the presence of the NEC8 wild-type cells or Claudin6 knock-out NEC8 cells. FIG. 9B shows the dose-dependent activity of the CLDN6/CD137 BsAbs 1912Ab3, 1912Ab4 and benchmark control Urelumab-NR in the presence of the NEC8 wild-type cells. FIG. 9C shows the IFNγ secretion by 1912Ab5 or Urelumab-NR in the co-culture signaling assay using NEC8 wild-type cells. FIG. 9D shows the NFKB activation by 1912Ab5 or Urelumab-NR in the co-culture signaling assay using Claudin6 knock-out NEC8 cells.

FIGS. 10A-B shows T cell-derived killing of target cell killing. FIG. 10A shows NEC8 cell killing by the CLDN6/CD137 bispecific antibodies 1912Ab3 and 1912Ab4. FIG. 10B shows T cell-derived OV90 cell killing by the CLDN6/CD137 bispecific antibodies 1912Ab5.

FIGS. 11A-C shows in vivo efficacy and safety data using a murine MC38 tumor model. FIG. 11A shows-Claudin 6 inhibition of MC38-Claudin 6 tumor growth in vivo by the CLDN6/CD137 BsAbs1912Ab3 and 1912Ab4. The mouse live enzyme activity was measured using day 21 serum. The ALT activity is shown in FIG. 11B, and the AST activity is shown in FIG. 11C. FIG. 11D shows the results from the rechallenge study using the mice previously treated by 1912Ab3 or 1912Ab4 and had complete tumor remission.

FIG. 12 shows the anti-tumor growth effect of 1912Ab5 at 0.3 mpk, 1 mpk, and 3 mpk.

FIG. 13 shows the anti-tumor growth effect of 1912Ab5 at 0.1 mpk and benchmark antibody Urelumab-NR at 0.1 mpk

FIG. 14 shows the anti-tumor effect of 1912Ab5 treating established large tumor

FIG. 15 shows fluorescent immunohistochemistry (IHC) data of control (FIG. 15A) or 1912Ab5 (FIG. 15B) treated tumors.

FIGS. 16A-F shows tumor infiltrated lymphocyte results of control or 1912Ab5 treated tumors. The data describes immune cell profiling comparing data of control and 1912Ab5 treated cells in CD4 (FIG. 16A), CD8 (FIG. 16B), T cem (FIG. 16C), Trm (FIG. 16D), exhausted T cells (FIG. 16E) and M2-like macrophage cells (FIG. 16F).

FIG. 17 shows the anti-tumor growth effect of 1912Ab5 in treating B16-F10 tumors.

FIG. 18 shows the binding activity of monospecific 1901Ab1 and CLDN18.2/CD137 BsAbs 1901Ab2 and 1901Ab3 to human Claudin 18.2 on NUGC4 cells.

FIG. 19 shows the binding activity of CLDN18.2/CD137 BsAbs 1901Ab2 and 1901Ab3, and monospecific anti-CD137 antibody 1923Ab4 to human CD137 on a cell surface.

FIGS. 20A-B show Claudin18.2 dependent activation of CD137 signaling by CLDN18.2/CD137 BsAbs using Jurkat T cell CD137 reporter cells. FIG. 20A shows the bar graph and FIG. 20B shows the dose dependence activity of Claudin18.2-CD137 bispecific antibodies.

FIG. 21 shows dose-response curves of CLDN18.2/CD137 BsAbs to induce CD8 T cell activation in the presence of NUGC4 cells.

FIG. 22 shows T cell-derived target cell killing by the CLDN18.2/CD137 BsAbs 1901Ab2 and 1901Ab3.

FIG. 23 shows inhibition of MC38-Claudin18.2 tumor growth in vivo by CLDN18.2/CD137 BsAb 1901Ab2.

FIG. 24 shows the binding activity of Nectin4/CD137 BsAbs 1925Ab1, 1925Ab2, and 1925Ab3 to human Nectin4 on CHO cells compared to the binding activity of the parental murine monoclonal antibody 1925Ab4.

FIG. 25 shows the binding activity of the Nectin4/CD137 BsAbs 1925Ab1, 1925Ab2 and 1925Ab3 to human CD137 on a cell surface compared to the binding activity of the parental murine monoclonal antibody 1925Ab4.

FIGS. 26A-B show that Nectin-4/CD137 BsAbs induce target cell-dependent CD137 agonism using Jurkat T cell CD137 reporter cells. FIG. 26A shows the bar graph and FIG. 26B shows the dose dependence activity of Nectin4/CD137 bispecific antibodies.

FIGS. 27A-C show the immune cell infiltration induced by control antibody, Urelumab-NR or 1912Ab5 treatment. Mouse liver IHC sections stained by CD4 (A), CD8 (B) and F4/80 (C) were used to show the T cell infiltration and macrophage infiltration.

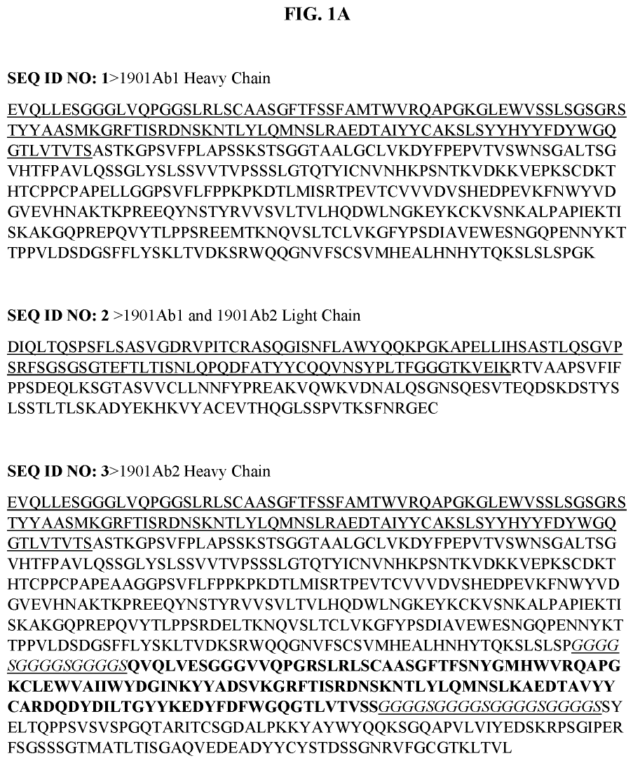

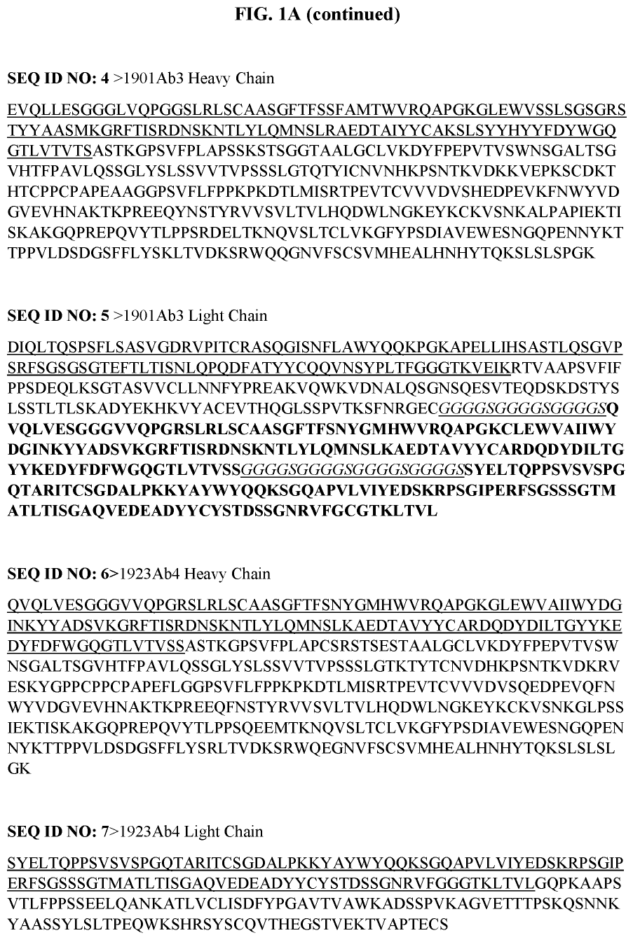

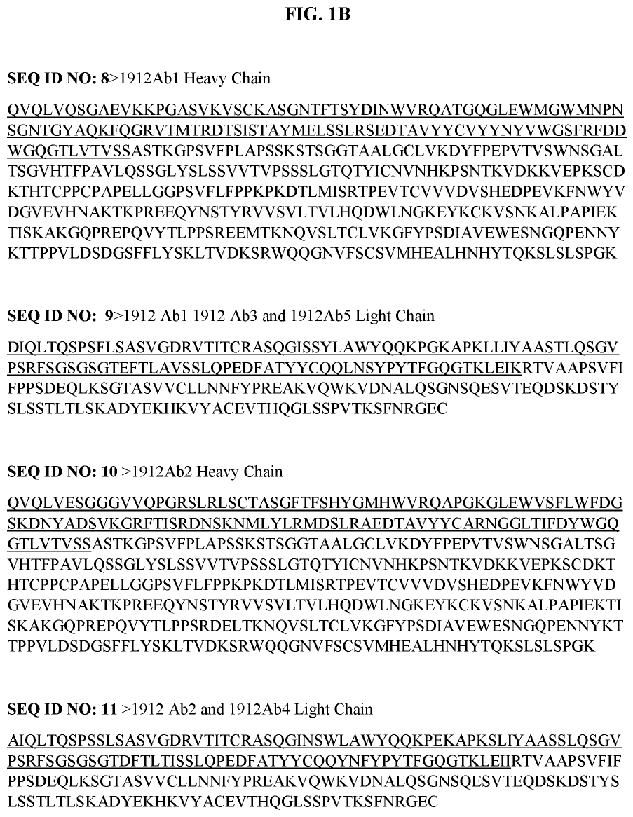

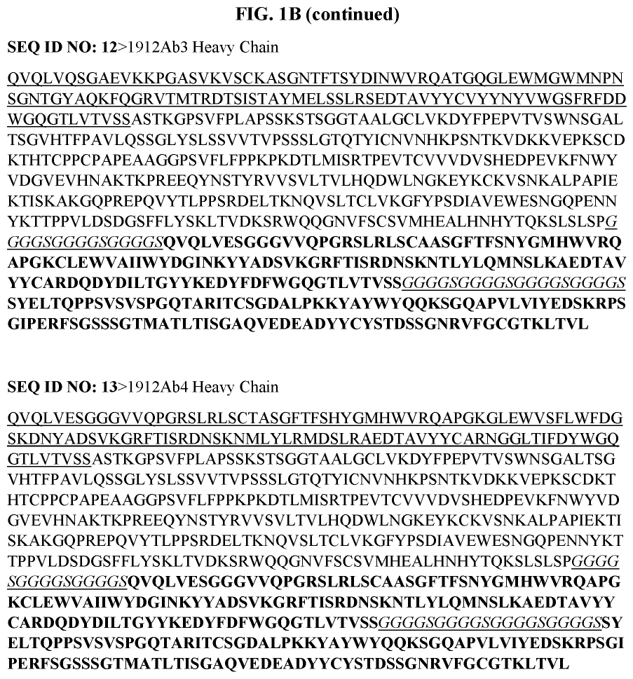

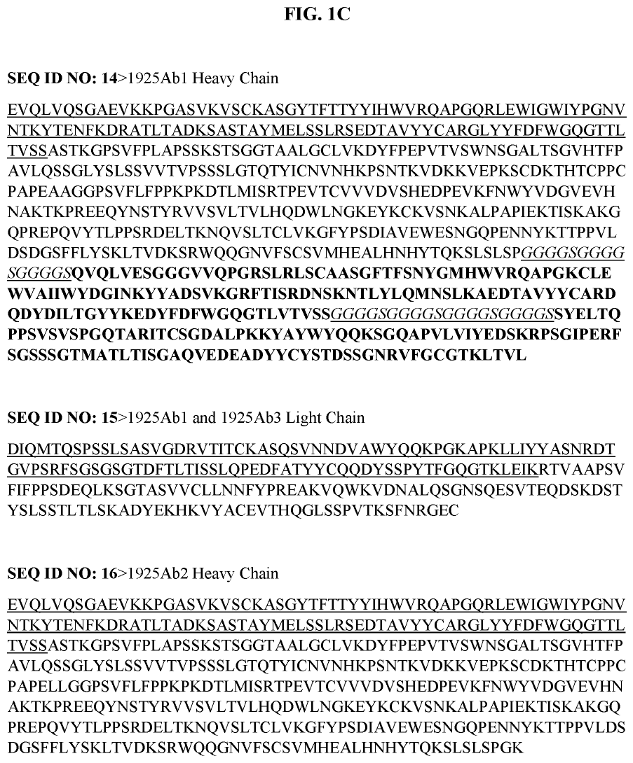

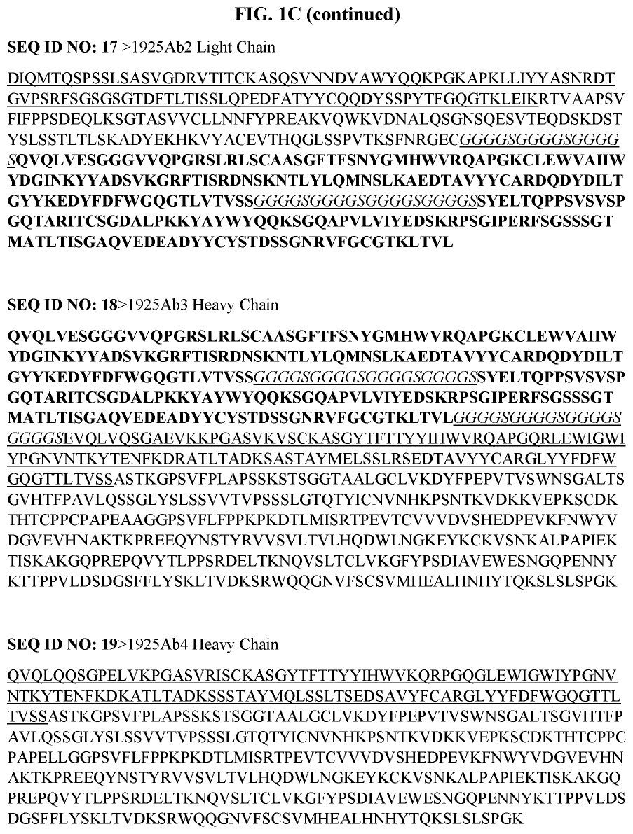

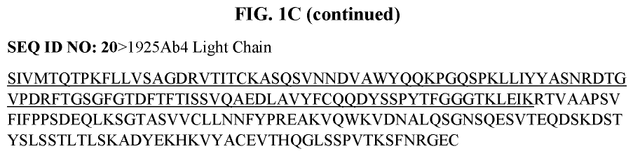

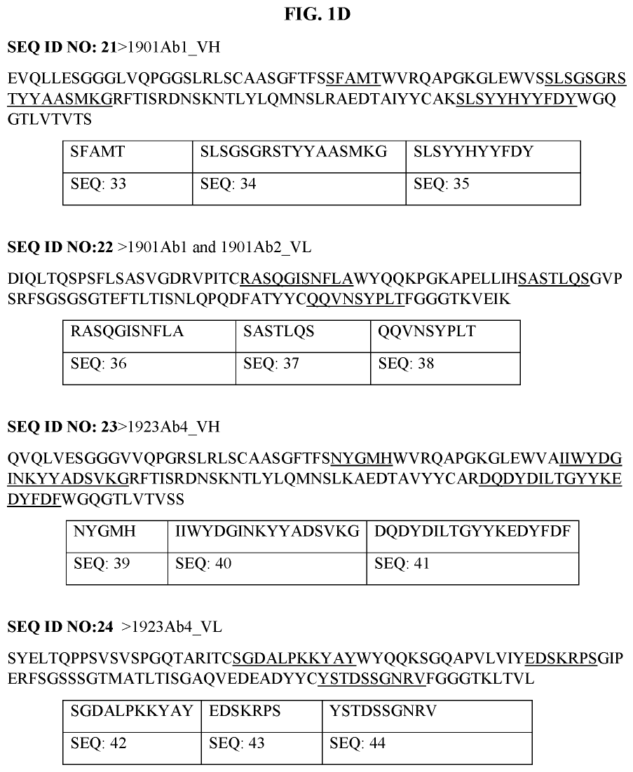

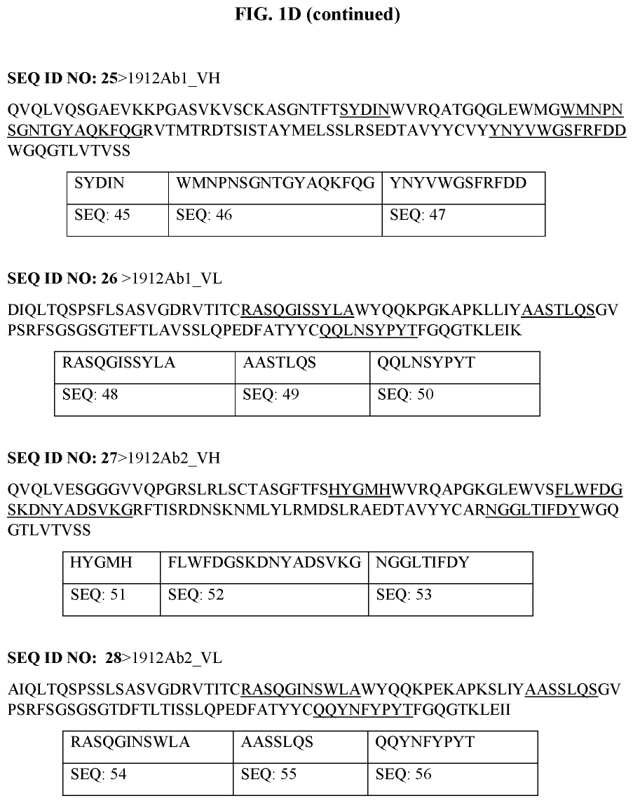

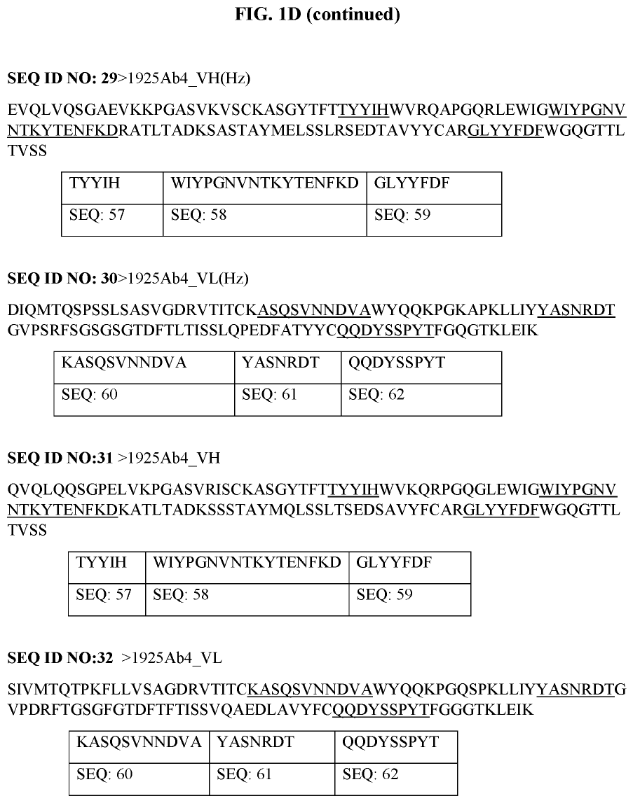

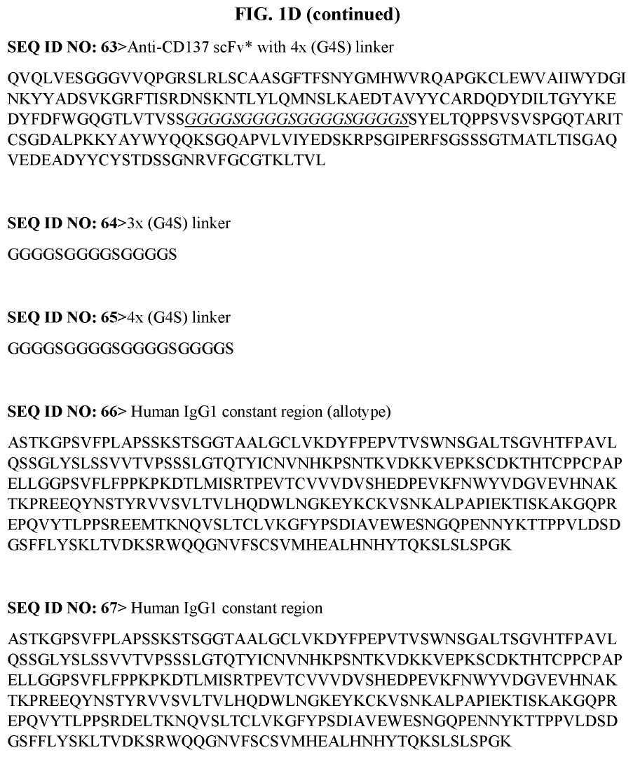

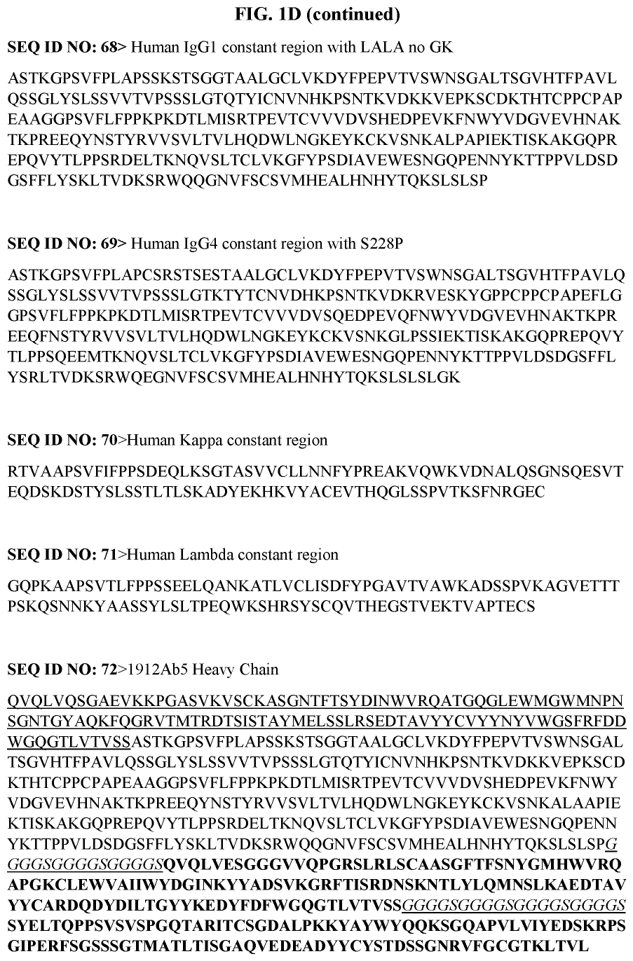

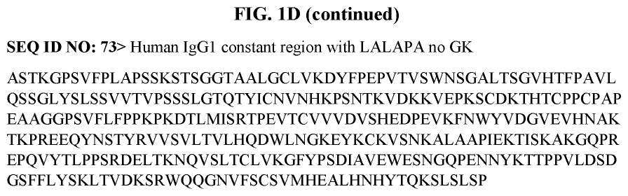

### DETAILED DESCRIPTION

The present disclosure provides bispecific binding proteins that bind CD137 and a tumor associated antigen (TAA). Exemplary TAAs include, but are not limited to Claudin 6, Claudin 18.2, and Nectin 4. Advantageously, the bispecific binding proteins disclosed herein are able to overcome on-target toxicity. For example, in a tissue, such as liver, where a tumor associated antigen is not expressed or accessible, the molecules of the present disclosure would be safe as they cannot activate CD137-mediated cytotoxicity. In a tumor tissue wherein a tumor associated antigen is overexpressed or accessible, by contrast, the antibodies undergo tumor associated antigen binding-dependent CD137 signaling activation, leading to CD137-mediated immune cell activation, thereby treating the tumor. The bispecific binding proteins can be used for the treatment of cancer. Additionally, the bispecific binding proteins disclosed herein result in lower dose formulations, resulting in less frequent and/or more effective dosing, and lead to reduced cost and increased efficiency.

In the tumor microenvironment, full T cell activation relies on two signals: one is mediated through TCR/CD3 activation, the other is mediated by a co-stimulatory pathway. Among the surface receptors that provide T cell co-stimulation, CD137 is an important regulator. Tumor-targeting CD137 agonistic antibody can be used alone or in combination with tumor-targeting CD3 agnostic antibody to promote T cell proliferation, survival, memory formation, and tumor-killing function.

CD137 co-stimulation (i.e., agonism) has been reported to lead to extended T-cell proliferation, reactivating anergic T cells, promoting memory T cell formation and maintenance (Hashimoto K. Cancers (Basel). 2021 May 11; 13 (10): 2288; Chester C et al., Blood 2018 Jan. 4; 131 (1): 49-57)). Activating CD137 with agonistic antibodies provides an opportunity to improve the therapeutic efficacy of immune checkpoint inhibitors (ICIs) or overcome resistance to ICIs. Additionally, a bispecific antibody that activates CD137 signaling only in the presence of TAA could help reduce the dose-dependent hepatotoxicity observed in clinical trials with monoclonal anti-CD137 agonistic antibody to the activation of CD137 signaling in liver resident Kupffer cells. Accordingly, the disclosure provides novel tetravalent TAA/CD137 binding proteins (i.e., bispecific antibodies) uniquely designed to activate the CD137 co-stimulatory pathway in the tumor microenvironment through TAA-mediated clustering of CD137.

So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

Throughout this disclosure, the following abbreviations will be used:
BsAb—Bispecific antibody.
mAb or Mab or MAb—Monoclonal antibody.
CDR—Complementarity determining region in the immunoglobulin variable regions.
VH or VH—Immunoglobulin heavy chain variable region.
VL or VL—Immunoglobulin light chain variable region.
FR—Antibody framework region, the immunoglobulin variable regions excluding the CDR regions.

The term “CD137” refers to 4-1BB, or TNFRSF9 (TNF Receptor Superfamily Member 9), a member of the TNF-receptor superfamily (TNFRSF) and is a co-stimulatory molecule which is expressed following the activation of immune cells (both innate and adaptive immune cells). As used herein, 4-1BB may be originated from a mammal, for example, Homo sapiens (human) (NCBI Accession No. NP_001552). As described herein, the term CD137 includes variants, isoforms, homologs, orthologs, and paralogs. For example, antibodies specific to a human CD137 protein may, in certain cases, cross-react with a CD137 protein from a species other than human. In other embodiments, the antibodies specific for a human CD137 protein may be completely specific for the human CD-137 protein and may exhibit species or other types of cross-reactivity, or may cross-react with CD137 from certain other species but not all other species (e.g., cross-react with monkey CD137, but not mouse 4-1BB). The term “cyno CD137” refers to cynomolgus monkey CD137, such as the complete amino acid sequence having NCBI Accession No. XP 005544945.1. The term “mouse CD137” refers to mouse sequence 4-1BB, such as the complete amino acid sequence of mouse 4-1BB having NCBI Accession No. NP_035742.1. The human CD137 sequence in the disclosure may differ from the human CD137 of NCBI Accession No. NP_001552 by having, e.g., conserved mutations or mutations in non-conserved regions, and the CD137 in the disclosure has substantially the same biological function as the human CD137 of NCBI Accession No. NP 001552.

The term “tumor associated antigen” or “TAA” refers to an antigen that is expressed on the surface of a tumor cell in a higher amount (e.g., 10%, 20%, 30%, 40%, 50%, 60%, 70%, 80%, 90%, 100% or greater amount) than is observed on normal cells (i.e., non-tumor cells). The term “tumor specific antigen” or “TSA” refers to antigens unique to tumors. Non-limiting examples of TAA and TSA include AFP, BAGE, BCMA, Claudin6, Claudin18.2, CAMEL, CEA, CD19, CD20, CD22, CD30, CD38, CD71, CD123, CD133, DAM-6, GPRC5D, PCMA, EGFR, cMET, HER2, HER3, TROP2, ROR1, ROR2, MSLN, B7H3, B7H4, PD-L1, MAGE, MUC1, MUC16, NY-ESO-1, PSM, TRP-2, Wt-1, PSA and SART-1.

The terms “Claudin 6” or “CLDN6” (used interchangeably herein) preferably relates to human CLDN6 and, in particular, to a protein comprising the amino acid sequence according to SEQ ID NO: 75 of the sequence listing or a variant of said amino acid sequence. The term “CLDN6” includes any CLDN6 variants such as post-translationally modified variants and conformation variants. The amino acid sequences for human, cynomolgus, and murine CLDN6 are provided in NCBI Reference Sequences: NP_067018.2 (human) (SEQ ID NO: 75), XP_005591080.1 (cynomolgus monkey (SEQ ID NO: 76), and NP_061247.1 (mouse) (SEQ ID NO: 77). Orthologs of CLDN6 share>99% and ˜88% identity to the human protein in cynomolgus monkeys and mice, respectively.

As used herein, the term “claudin 18 isoform 2” (used interchangeably with CLDN18.2) refers to a peptide comprising or consisting of the amino acid sequence provided in NCBI entry NP_001002026.1, Claudin-18 isoform 2 including post-translationally modified variants and species homologs present on the surface of normal or transformed cancer cells or are expressed on cells transfected with a CLDN18.2 gene. Claudin 18.2 preferably has the amino acid sequence according to SEQ ID NO: 72.

The term “Nectin-4” (N4), or “Nectin-4 protein” includes human Nectin-4, in particular the native-sequence polypeptide, isoforms, chimeric polypeptides, all homologs, fragments, and precursors of Nectin-4. The amino acid sequences for human, cynomolgus, rat and murine Nectin-4 are provided in NCBI Reference Sequences: NP_112178.2 (human) (SEQ ID NO: 78), XP_005541277.1 (cynomolgus monkey (SEQ ID NO: 79), NP_001102546.1 (rat) (SEQ ID NO: 80), and NP_082169.2 (mouse) (SEQ ID NO: 81). Orthologs of Nectin-4 share>99%, ˜94% and ˜92% homology to the human protein in cynomolgus monkey, rat and mouse, respectively.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman: 1981, Ads App. Math. 2, 482, by means of the local homology algorithm of Neddiernan and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The term “antibody” herein is used in the broadest sense and encompasses various antibody structures, including but not limited to monoclonal antibodies, polyclonal antibodies, and multispecific antibodies (e.g., bispecific antibodies).

The term “antibody scaffold module” herein refers to a Y-shaped antibody having two heavy and two light chains. The two heavy chains are linked to each other by disulfide bonds and each heavy chain is linked to a light chain by a disulfide bond. An antibody scaffold may have one or more binding modules attached to one or more of its heavy and/or light chains. The antibody binding scaffold comprises two Fabs and a Fc portion having two constant region sequences.

The term “cross-reacts,” as used herein, refers to the ability of anti-human CD137 or TAA antibody described herein to bind to CD137 or TAA, respectively, from a different species. For example, an antibody described herein may also bind CD137 or TAA from another species (e.g., rat or mouse CD137 or TAA).

An exemplary antibody such as an IgG comprises two heavy chains and two light chains. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL are composed of three CDRs and four FRs, arranged from amino terminus to carboxy-terminus in the following order: FR1, CDR1, FR2, CDR2, FR3, CDR3, FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917.

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring the production of the antibody by any method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to a recombinant antibody in which a portion of the heavy and/or light chain is identical with or homologous to corresponding sequences in antibodies derived from a particular species, or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. In addition, complementarity determining region (CDR) grafting may be performed to alter certain properties of the antibody molecule including affinity or specificity. Typically, the variable domains are obtained from an antibody from an experimental animal (the “parental antibody”), such as a rodent, and the constant domain sequences are obtained from human antibodies, so that the resulting chimeric antibody can direct effector functions in a human subject and will be less likely to elicit an adverse immune response than the parental (e.g., mouse) antibody from which it is derived.

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al., Monoclonal Antibodies and Cancer Therapy, Alan R. Liss, p. 77 (1985); Boerner et al., J. Immunol, 147 (I): 86-95 (1991). See also van Dijk and van de Winkel, Curr. Opin. Pharmacol, 5:368-74 (2001). Human antibodies can be prepared by administering the antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., lgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546), which consist of a VH domain; (vi) isolated complementarity determining regions (CDR), and (vii) combinations of two or more isolated CDRs which may optionally be joined by a synthetic linker.

The “variable domain” (V domain) of an antibody mediates binding and confers antigen specificity of a particular antibody. However, the variability is not evenly distributed across the 110-amino acid span of the variable domains. Instead, the V regions consist of relatively invariant stretches called framework regions (FRs) of 15-30 amino acids separated by shorter regions of extreme variability referred to herein as “hypervariable regions” or CDRs that are each 9-12 amino acids long. As will be appreciated by those in the art, the exact numbering and placement of the CDRs can be different among different numbering systems. However, it should be understood that the disclosure of a variable heavy and/or variable light sequence includes the disclosure of the associated CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3).

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH. Unless stated otherwise herein, CDR and framework regions are annotated according to the Kabat numbering scheme (Kabat E. A. et al., 1991, Sequences of Proteins of Immunological Interest, In: NIH Publication No. 91-3242, US Department of Health and Human Services, Bethesda, Md).

In other embodiments, the CDRs of an antibody can be determined according to MacCallum R M et al, (1996) J Mol Biol 262:732-745, herein incorporated by reference in its entirety. In other embodiments, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety. CDRs may also be defined by sequence comparison in Kabat et al., 1991, In: Sequences of Proteins of Immunological Interest, 5th Ed. Public Health Service, National Institutes of Health, Bethesda, Md., whereas HVLs are structurally defined according to the three-dimensional structure of the variable domain, as described by Chothia and Lesk, 1987, J. Mol. Biol. 196:901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. IMGT and NORTH provide alternative definitions of the CDRs (see, Lefranc M P. Unique database numbering system for immunogenetic analysis. Immunol Today (1997) 18:509; and North B, Lehmann A, Dunbrack R L J. A new clustering of antibody CDR loop conformations. J Mol Biol. (2011) 406:228-56). Additionally, CDRs may be defined per the Chemical Computing Group (CCG) numbering (Almagro et al., Proteins 2011; 79:3050-3066 and Maier et al., Proteins 2014; 82:1599-1610). The CDR1, CDR2, CDR3 of the heavy and light chains therefore define the unique and functional properties specific to a given antibody.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup Ill as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, the numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

The term “antibody fragment” refers to a molecule other than an intact antibody that comprises a portion of an intact antibody that binds to the antigen to which the intact antibody binds. Examples of antibody fragments include but are not limited to Fv, Fab, Fab′, Fab′-SH, F(ab)₂; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH) and the first constant domain of one heavy chain (CH1). Pepsin treatment of an antibody yields a single large F(ab)₂ fragment which roughly corresponds to two disulfide-linked Fab fragments having divalent antigen-binding activity and are still capable of cross-linking antigen. Fab fragments differ from Fab′ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” consists of a dimer of one heavy- and one light-chain variable region domain in a tight, non-covalent association. From the folding of these two domains emanate six hypervariable loops (3 loops each from the H and L chain) that contribute to the amino acid residues for antigen binding and confer antigen binding specificity to the antibody.

A “single-chain variable fragment” or “scFv” refers to a fusion protein of the variable regions of the heavy (V_{H}) and light chains (V_{L}) of immunoglobulins. For a review of sFv, see Plückthun in The Pharmacology of Monoclonal Antibodies, vol. 113, Rosenburg and Moore eds., Springer-Verlag, New York, pp. 269-315 (1994). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removing the constant regions and introducing the linker. Disulfide-stabilized scFv can be engineered by introducing paired cysteine mutating specific VH or VL residues. These residues are at the interface of VH and VL. Please see reference Weatherill, E. E. et al. Towards a universal disulphide stabilized single chain Fv format: importance of interchain disulphide bond location and VL-VH orientation. Protein Eng Des Sel 25, 321-329, NovaRock used VH44-VL100.

The term “multispecific antibody” is used in the broadest sense and specifically covers an antibody comprising a heavy chain variable domain (VH) and a light chain variable domain (VL), where the VH-VL unit has polyepitopic specificity (e.g., is capable of binding to two different epitopes on one biological molecule or each epitope on a different biological molecule). Such multispecific antibodies include, but are not limited to, full-length antibodies, antibodies having two or more VL and VH domains, bispecific diabodies and triabodies. “Polyepitopic specificity” refers to the ability to specifically bind to two or more different epitopes on the same or different target(s).

“Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody in an IgG1 form binds to each epitope with an affinity of 5 μM to 0.001 pM, 3 μM to 0.001 pM, 1 μM to 0.001 pM, 0.5 μM to 0.001 pM or 0.1 μM to 0.001 pM. “Monospecific” refers to the ability to bind only one epitope. Multi-specific antibodies can have structures similar to full immunoglobulin molecules and include Fc regions, for example, IgG Fc regions. Such structures can include, but are not limited to, IgG-Fv, IgG-(scFv)₂, DVD-Ig, (scFv)₂-(scFv)₂-Fc and (scFv)₂-Fc-(scFv)₂. In the case of IgG-(scFv)₂, the scFv can be attached to either the N-terminal or the C-terminal end of either the heavy chain or the light chain.

As used herein, the term “bispecific antibodies” (BsAb) refers antibodies, often human or humanized, that have binding specificities for at least two different antigens. In the disclosure, one of the binding specificities can be directed towards CD137 and the other for CLDN6, CLDN18.2, or Nectin-4.

As used herein, the term “diabodies” refers to bivalent antibodies comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain.

The term “isolated antibody” when used to describe the various antibodies disclosed herein, means an antibody that has been identified and separated and/or recovered from a cell or cell culture from which it was expressed. An isolated antibody or antibody fragment may include variants of the antibody or antibody fragment having one or more post-translational modifications that arise during production, purification, and/or storage of the antibody or antibody fragment. Contaminant components of its natural environment are materials that would typically interfere with diagnostic or therapeutic uses for the polypeptide. They can include enzymes, hormones, and other proteinaceous or non-proteinaceous solutes. In some embodiments, an isolated antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87 (2007). In a preferred embodiment, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining the binding of a molecule compared to the binding of a control molecule. For example, specific binding can be determined by competition with a control molecule similar to the target, such as an excess of a non-labeled target. In this case, specific binding is indicated if the binding of the labeled target to a probe is competitively inhibited by the excess unlabeled target. The term “specific binding” or “specifically binds to” or is “specific for” a particular polypeptide or an epitope on a particular polypeptide target as used herein can be exhibited, for example, by a molecule having a Kd for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a Kd in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to CD137, CLDN6, CLDN18.2, or Nectin-4 (or to a CD137, CLDN6, CLDN18.2, or Nectin-4 epitope) without substantially binding to any other polypeptide or polypeptide epitope.

As used herein the term “binds CD137”, “binds CLDN6”, “binds CLDN18.2”, “binds Nectin-4” refers to the ability of an antibody, or antigen-binding fragment to recognize and bind endogenous human CD137, CLDN6, CLDN18.2, or Nectin-4, respectively, as it occurs on the surface of normal or malignant cells or on the surface of recombinant host cells engineered to overexpress CD137, CLDN6, CLDN18.2, or Nectin-4, respectively.

The term “affinity,” as used herein, means the strength of the binding of an antibody to an epitope. The affinity of an antibody is given by the dissociation constant Kd, defined as [Ab]×>[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

An “epitope” is a term of art that indicates the site or sites of interaction between an antibody and its antigen(s). As described by (Janeway, C, Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein . . . [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of the polypeptide chain is termed a continuous or linear epitope” (Janeway, C. Jr., P. Travers, et al. (2001). Immunobiology: the immune system in health and disease. Part II, Section 3-8. New York, Garland Publishing, Inc.).

The term “KD”, as used herein, refers to the equilibrium dissociation constant, which is obtained from the ratio of kd to ka (e.g., kd/ka) and is expressed as a molar concentration (M). KD values for antibodies can be determined using methods well established in the art. Preferred methods for determining the KD of an antibody include biolayer interferometry (BLI) analysis, preferably using a Fortebio Octet RED device, surface plasmon resonance, preferably using a biosensor system such as a BIACORE® surface plasmon resonance system, or flow cytometry and Scatchard analysis.

The term “KD”, as used herein, is intended to refer to the dissociation constant of a particular antibody-antigen interaction. It is calculated by the formula: Koff/Kon=KD. Binding kinetics describe how fast a antibody binds to its target (Kon) and how fast it dissociates from it (Koff). Antibody residence time on its target, for example, CD137 is determined by these kinetic features (Schuetz, D A, et al. (2017) Kinetics for Drug Discovery: an industry-driven effort to target drug residence time. Drug Discov Today 22:896-911).

The term “IC₅₀”, as used herein, is intended to refer to the effective concentration of a bispecific binding protein disclosed herein needed to neutralize 50% of the bioactivity of an antigen to which it binds.

“EC₅₀” with respect to an agent and a particular activity (e.g., binding to a cell, inhibition of enzymatic activity, activation or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

As used herein the term “antibody-drug conjugate” (ADC) refers to immunoconjugates consisting of recombinant monoclonal antibodies covalently linked to cytotoxic agents (known as payloads) via synthetic linkers. Immunoconjugates (Antibody-drug conjugates, ADCs) are a class of highly potent antibody-based cancer therapeutics. ADCs consist of recombinant monoclonal antibodies covalently linked to cytotoxic agents (known as payloads) via synthetic linkers. ADCs combine the specificity of monoclonal antibodies and the potency of small-molecule chemotherapy drugs, and facilitate the targeted delivery of highly cytotoxic small molecule drug moieties directly to tumor cells.

As used herein the term “endocytosis” refers to the process where eukaryotic cells internalize segments of the plasma membrane, cell-surface receptors, and components from the extracellular fluid. Endocytosis mechanisms include receptor-mediated endocytosis. The term “receptor-mediated endocytosis” refers to a biological mechanism by which a ligand, upon binding to its target, triggers membrane invagination and pinching, gets internalized and delivered into the cytosol or transferred to appropriate intracellular compartments.

The term “bystander effect” refers to target-cell mediated killing of healthy cells adjacent to tumor cells targeted for by an antibody drug conjugate. The bystander effect is generally caused by cellular efflux of hydrophobic cytotoxic drugs, capable of diffusing out of an antigen-positive target cell and into adjacent antigen-negative healthy cells. The presence or absence of the bystander effect can be attributed to aspects of the linker and conjugation chemistries used to produce an immunoconjugate.

The term “effector functions,” deriving from the interaction of an antibody Fc region with certain Fc receptors, include but are not necessarily limited to C1q binding, complement dependent cytotoxicity (CDC), Fc receptor binding, FcyR-mediated effector functions such as ADCC, antibody dependent cell-mediated phagocytosis (ADCP), T cell dependent cellular cytotoxicity (TCDD) and down regulation of a cell surface receptor. Such effector functions generally require the Fc region to be combined with an antigen binding domain (e.g., an antibody variable domain).

As used herein the terms “antibody-based immunotherapy” and “immunotherapies” are used to broadly refer to any form of therapy that relies on the targeting specificity of binding protein that binds CD137 and CLDN6, CD137 and CLDN18.2, or CD137 and Nectin-4, to mediate a direct or indirect effect on a CD137, CLDN6, CLDN18.2, and/or Nectin-4 expressing cell.

The term “Fc receptor” or “FcR” describes an antibody receptor that binds to the Fc region of an immunoglobulin, which is involved in antigen recognition located at the membrane of certain immune cells including B lymphocytes, natural killer cells, macrophages, neutrophils, and mast cells. Fc receptors recognizing the Fc portion of IgG are called Fc gamma receptors (FcγRs). The FcγR family includes allelic variants and alternatively spliced forms of these receptors. Based on the differences in structure, function, and affinity for IgG binding, FcγRs are classified into three major groups: FcγRI, FcγRII (FcγRIIa and FcγRIIb) and FcγRIII (FcγRIIIa and FcγRIIIb). Among them, FcγRI (CD64), FcγRIIa (CD32a), and FcγRIIIa (CD16a) are activating receptors containing the signal transduction motif, immunoreceptor tyrosine-based activation motif (ITAM), in the γ subunit of FcγRI and FcγRIIIa, or in the cytoplasmic tail of FcγRIIa. After binding of antigen-antibody complexes the activatory Fcγ receptors (human: FcγRI, FcγRIIA, FcγRIIC, FcγRIIIA, FcγRIIIB and murine: FcγRI, FcγRIII, FcγRIV) trigger immune effector functions. In contrast, FcγRIIb (CD32b) is an inhibitory receptor. Cross-linking of FcγRIIb leads to the phosphorylation of the immunoreceptor tyrosine-based inhibitory motif (ITIM) and inhibitory signaling transduction (Patel et al. Front Immunol. 2019; 10:223.).

The term “Fc silenced” refers to the Fc region that is engineered to minimize/abolish binding activity with FcγRs and complement, leading to silence or eliminate of Fc-mediated effector functions. The strategies for engineering Fc include modification of Fc glycosylation, using hybrid of IgG subclasses, or introducing one or more mutations in the hinge and/or CH2 regions. The residues are important for effector functions and respective mutations that silence Fc are known in the art, for example, Strohl, WR and Strohl L M, “Antibody Fc engineering for optimal antibody performance” In Therapeutic Antibody Engineering, Cambridge: Woodhead Publishing (2012), pp 242, International Patent Publication No. WO 2017/008169A1 and WO 2021/055669.

Specific, non-limiting examples for sites that can be engineered to silence human IgG1 Fc include L234, L235, G237, D265, N297, P329, P331, all in EU numbering.

As used herein, the term “bispecific” refers to binding proteins comprising an antibody scaffold module and a first binding module, wherein the modules are derived from antibodies and/or receptor proteins that have binding specificities for two different antigens. In one embodiment, the antibody scaffold module has binding specificity for a tumor associated antigen (TAA), and the first binding module has binding specificity for CD137 (e.g., human CD137).

With regard to the binding of a bispecific binding protein to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10-10⁻¹⁰ M or lower, alternatively 10⁻¹¹ M or lower, alternatively 10⁻¹² M or lower or a Kd in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “affinity,” as used herein, means the strength of the binding of a bispecific binding protein to an epitope. The affinity of an bispecific binding protein is given by the dissociation constant Kd, defined as [bispecific binding protein]×[Ag]/[bispecific binding protein−Ag], where [bispecific binding protein−Ag] is the molar concentration of the bispecific binding protein-antigen complex, [bispecific binding protein] is the molar concentration of the unbound bispecific binding protein and [Ag] is the molar concentration of the unbound antigen. The affinity constant Ka is defined by 1/Kd. Methods for determining the affinity of binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of bispecific binding proteins is the use of surface plasmon resonance (SPR) screening (such as by analysis with a BIAcore™ SPR analytical device).

The term “linker” refers to at least one atom that forms a covalent bond between two chemical entities. The term “linker” may refer to at least one atom that forms a covalent bond between the scaffold module and another covalent bond to the binding module. If the scaffold module and binding module are linked solely through peptide bonds, the linker is referred to as a “peptide linker”. Otherwise, the linker is referred to as a “chemical linker”. Further, a “flexible peptide linker” comprises mostly small, non-polar or polar amino acids whereas a “rigid peptide linker” comprises alpha-helix forming sequences and/or are rich in proline residues (Chen et al., 2013. Adv Drug Deliv Rev. 65 (10): 1357-1369).

### CD137 (4-1BB)

CD137 (4-1BB) is an inducible costimulatory receptor expressed on activated T and natural killer (NK) cells. The 4-1BB protein has four extracellular cysteine-rich pseudo repeats (CRD) domains, CRD1, CRD2, CRD3 and CRD4 (see the amino acid sequence and the CRD regions in the table below). 4-1BB trimer clustering by 4-1BB ligand (41 BBL) trimer on T cells triggers a signaling cascade that results in upregulation of antiapoptotic molecules, cytokine secretion, and enhanced effector function. On NK cells, 4-1BB signaling can increase antibody-dependent cell-mediated cytotoxicity.

CD137, a member of the TNF receptor superfamily, was first identified as an inducible molecule expressed by activated by T cells (Kwon and Weissman, 1989, Proc Natl Acad Sci USA 86, 1963-1967). Subsequent studies demonstrated that many other immune cells also express 4-1BB, including NK cells, B cells, NKT cells, monocytes, neutrophils, mast cells, dendritic cells (DCs) and cells of non-hematopoietic origin such as endothelial and smooth muscle cells (Vinay and Kwon, 2011, Cell Mol Immunol 8, 281-284). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002, J Immunol 168, 3755-3762; Zhang et al., 2010, Clin Cancer Res 13, 2758-2767).

4-1BB ligand (4-1BBL or CD137L) was identified in 1993 (Goodwin et al., 1993, Eur J Immunol 23, 2631-2641). It has been shown that expression of 4-1BBL was restricted on professional antigen presenting cells (APC) such as B-cells, DCs and macrophages. Inducible expression of 4-1BBL is characteristic of T-cells, including both αβ and γδ T-cell subsets, and endothelial cells (Shao and Schwarz, 2011, J Leukoc Biol 89, 21-29).

Co-stimulation through the 4-1BB receptor (for example by 4-1BBL ligation) activates multiple signaling cascades within the T cell (both CD4 and CD8 subsets), powerfully augmenting T cell activation (Bartkowiak and Curran, 2015). In combination with TCR triggering, agonistic 4-1BB-specific antibodies enhance the proliferation of T-cells, stimulate lymphokine secretion and decrease sensitivity of T-lymphocytes to activation-induced cells death (Snell et al., 2011, Immunol Rev 244, 197-217). This mechanism was further advanced as the first proof of concept in cancer immunotherapy. In a preclinical model, administration of an agonistic antibody against 4-1BB in tumor bearing mice led to a potent anti-tumor effect (Melero et al., 1997, Nat Med 3, 682-685). Later, accumulating evidence indicated that 4-1BB usually exhibits its potency as an anti-tumor agent only when administered in combination with other immunomodulatory compounds, chemotherapeutic reagents, tumor-specific vaccination or radiotherapy (Bartkowiak and Curran, 2015, Front Oncol 5, 117).

Agonistic monoclonal antibodies targeting 4-1BB have been developed to harness 4-1BB signaling for cancer immunotherapy. Preclinical results in a variety of induced and spontaneous tumor models suggest that targeting 4-1BB with agonist antibodies can lead to tumor clearance and durable antitumor immunity. Additionally, fusion proteins composed of one extracellular domain of a 4-1BB ligand and a single chain antibody fragment (Homig et al., 2012, J Immunother 35, 418-429; Muller et al., 2008, J Immunother 31, 714-722) or a single 4-1BB ligand fused to the C-terminus of a heavy chain (Zhang et al., 2007, Clin Cancer Res 13, 2758-2767) have been made. WO 2010/010051 discloses the generation of fusion proteins that consist of three TNF ligand ectodomains linked to each other and fused to an antibody part.

The first generation of immune agonist CD137 antibodies such as Urelumab and Utomilumab have not achieved the desired efficacy in the clinic. For T cell costimulate agonists to work as cancer therapies, many factors need to be considered: the target-engaging affinity, binding kinetics, binding valency, clustering formation, Fc receptor-mediated activities, etc. A fit-for-purpose tumor antigen-CD137 configuration design has been used for improving the potency and safety of the disclosed bispecific antibodies. Specifically, highly specific tumor antigen-binding antibodies were selected for tumor cell engagement, bi-valency for tumor antigen binding was used to maximize target engagement. 2) Sub-optimal activation of T cells was considered to have less T-cell exhaustion and a long-lasting anti-tumor effect (Stone J D et al., Immunology. 2009; 126 (2): 165-176). CD137 antibodies with fast-on, fast-off features were anticipated to avoid a constant stimulation signal to the T cell, therefore, work better than slow-off antibodies (Garble K, Nature Reviews Drug Discovery 19, 3-5 (2020). Additionally, CD137 agonism with clustering dependency can avoid the systemic activation of circulation T cells and only activate the Tumor cell experienced T cells at the tumor site. This was achieved by using a tumor antigen-clustering-dependent CD137 agonism. Furthermore, silencing Fc to eliminate effector function and Fc mediated receptor clustering can further reduce the Kuffer cell activation derived liver toxicity.

### Claudin Protein Family

The Claudin (CLDN) family is composed of 27 members and displays distinct expression patterns in cell- and tissue-type-selective manners. Claudins are integral membrane proteins located within the tight junctions (TJs) of epithelia and endothelia. CLDNs interact with each other, both in the same cell (cis-interaction) and on adjacent cells (trans-interaction), resulting in the constitution of TJs with tissue-specific barrier functions. Individual cell types express more than one of the claudin family members. In normal physiology, the claudins interact with multiple proteins and are intimately involved in signal transduction to and from the tight junction (Lal-Nag, M and Morin, P. J., Genome Biol 10:235, 2009).

CLDN proteins comprise four transmembrane (TM) helices (TM1, TM2, TM3, and TM4) and two extracellular loops (ELI and EL2). The extracellular loops of claudins from adjacent cells interact with each other to seal the cellular sheet and regulate paracellular transport between the luminal and basolateral spaces. The claudin protein structure is highly conserved among the different family members and CLDN6 comprises 220 amino acids, is 23 kDa in size and exhibits a claudin-typical protein structure.

The first claudin family of protein was first cloned and named in 1998 as crucial structural and functional components of tight junctions. As a family, claudins are a multigene family of tetra-transmembrane proteins involved in the barrier functions of epithelial and endothelial cells and the maintenance of the cytoskeleton (Furuse et al., J. Cell. Biol. 141 (7): 1539-50, 1998). Claudins are integral membrane proteins comprising a major structural protein of tight junctions, the most apical cell-cell adhesion junction in polarized cell types such as those found in epithelial or endothelial cell sheets.

The first extracellular domain (ECD) of a claudin protein typically consists of about 50 amino acids, while the second one is smaller having about 22 amino acids (Hashimoto, et al. Drug Discovery Today 21 (10): 1711-1718, 2016). The N-terminal end is usually very short (e.g., about four to ten amino acids) while the C-terminal end ranges from 21 to about 63 amino acids and is required for localization of the proteins in tight junctions.

The observation that tight junction permeability is often higher in tumor tissues than in normal tissues, has led to speculation that claudin proteins on tumor cells may be more accessible than in normal tissues with intact tight junctions. This observation also makes claudin proteins attractive targets for therapeutic cancer interventions.

The claudin family of proteins in humans is comprised of at least 27 members, ranging in size from 22-34 kDa. All claudins possess a tetraspanin topology in which both protein termini are located on the intracellular face of the membrane, resulting in the formation of two extracellular (EC) loops, EC1 and EC2. Typically, EC1 is about 50-60 amino acids in size and EC2 is smaller than EC1 and usually comprises approximately 25 amino acids. The EC loops mediate head-to-head homophilic, and for certain combinations of claudins, heterophilic interactions that lead to formation of tight junctions.

### Claudin-6

Unlike the majority of Claudin proteins that are broadly expressed, CLDN6 is characterized by selective expression (Hewitt, et al., BMC Cancer, 6:186, 2006). CLDN6 is an oncofetal tight junction molecule expressed in several types of embryonic epithelial cells.

Disturbance of tight junctions and dysregulation of tight junction molecules is a frequent hallmarks of cancer cells and frequently associated with malignant transformation. CLDN6 expression is aberrantly activated in various cancer types, including gastric, lung and ovarian adenocarcinomas, endometrial and embryonal carcinomas, pediatric tumors of the brain (e.g., atypical teratoid/rhabdoid tumors) and germ cell tumors (Hassimoto et al., J Pharmacol Exp Ther 368:179-186, 2019; Kojima et al., Cancers 2020, 12, 2748). Increased expression of CLDN6 in several human malignancies is associated with poor prognosis such as ovarian cancer and gastric cancer (Zavala-Zendejas V E, et al., Cancer Invest. 29:1-11. 2011; Wang L, et al. Diagn Pathol. 8:1902013.). Therefore, CLDN6 is a promising tumor-associated antigen (TAA) for tumor-targeting therapeutics such as CART and T cell engaging bispecific antibodies.

As a tumor-associated antigen it can be classified as a differentiation antigen due to its expression during the early stage of epidermal morphogenesis where it is crucial for epidermal differentiation and barrier formation. The distinct expression pattern of CLDN6 in cancer but not in normal adult tissues combined with its cell surface accessibility to antibodies qualifies CLDN6 as a promising target for diagnostic as well as immunotherapeutic approaches in a wide variety of cancer types.

There is a high degree of sequence conservation between CLDN6 to other claudin proteins. The high homology of CLDN6 with other Claudin proteins (e.g., CLDN9, CLDN4 and CLDN3) makes it difficult to provide CLDN6 antibodies which have properties suitable for therapeutic use such as specificity, affinity and safety.

CLDN6 is generally expressed in humans as a 220-amino acid precursor protein, the first 21 amino acids of which constitute the signal peptide. The amino acid sequence of the CLDN6 precursor protein is publicly available at the National Center for Biotechnology Information (NCBI) website as NCBI Reference Sequence NP 067018.2 and is provided herein as SEQ ID NO: 75.

Expression CLDN6 is highly expressed in germ cell tumors, including seminomas, embryonal carcinomas and yolk sac tumors, as well as in some cases of gastric adenocarcinomas, lung adenocarcinomas, ovarian adenocarcinomas, and endometrial carcinomas (Ushiku T et al., Histopathology 61 (6): 1043-1056, 2012, Hewitt K J, Agarwal R, Morin P. The claudin gene family: expression in normal and neoplastic tissues. BMC Cancer 2006; 6; 186; Micke, P. et al. (2014) Aberrantly activated Claudin-6 and 18.2 as potential therapeutic targets in non-small-cell lung cancer. Int. J. Cancer 135, 2206-2214; Lal-Nag, M. et al. (2012) Claudin-6: a novel receptor for CPE-mediated cytotoxicity in ovarian cancer. Oncogenesis 1, e33; Ben-David, U. et al. (2013) Immunologic and chemical targeting of the tight junction protein Claudin-6 eliminates tumorigenic human pluripotent stem cells. Nat. Commun. 4, 1992).

Human CLDN6 protein is very closely related to the human CLDN9 protein sequence in the extracellular domains (ECD), with >98% identity in ECD1 and >91% identity in ECD2. Human CLDN4 is also closely related to human CLDN6 in the ECD sequences, with >84% identity in ECD1 and >78% identity in ECD2. Monoclonal antibody (MAb) discovery against CLDN6 has been encumbered by the high homology of endogenously expressed Claudin-9 (CLDN9), which varies from CLDN6 by only 3 amino acids (2 in ECD1 and 1 in ECD2) in their extracellular domains. Deduced cynomolgus monkey protein ECD sequences for CLDN4, CLDN6, and CLDN9 proteins are 100% identical to the respective human ECD sequences. In addition, the Claudin-6 gene is highly conserved among different species, for example, human and murine genes exhibit 88% homology at DNA and protein levels.

### Claudin 18.2

Tight junction molecule claudin-18, another member of the claudin family of proteins is normally found in the cellular tight junctions of gastric mucosa and intestinal epithelium. Two alternatively spliced human claudin 18 transcript variants, encoding distinct isoforms that exhibit lung-restricted (CLDN18.1) and stomach-restricted (CLDN18.2) expression (Niimi et al., Mol. Cell. Biol. 21:7380-90, 2001), in a promoter-dependent manner, have previously been described. The primary protein sequences of the splice variants differ in the N-terminal portion that comprises the N-terminal intracellular region, first transmembrane region (TMD1), and extracellular loop one (ECL1). CLDN18.2 is one of a few members of the human claudin family with strict restrictions to one cell lineage (Tureci et al.). More specifically, it provides a highly selective gastric lineage (e.g., gastrocyte-specific) marker with an expression pattern that is restricted to short-lived differentiated epithelial cells and absent from the stem cell zone of gastric glands (Sahin et al., Clin. Cancer Res. 14 (23) 7624-7634, 2008).

CLDN18.2 is retained in malignant transformation and is expressed in a significant portion of primary tumors and their metastasis. Sahin et al. also reported that CLDN18.2, but not CLDN18.1, is frequently overexpressed in several different types of cancers, including pancreatic, stomach, esophageal, lung, and ovarian cancers. Therefore, in the context of cancer, CLDN18.2 does not remain restricted to the gastric cell lineage (Sahin et al.). Considered together, the findings of published reports establish that CLDN18.2 provides both a diagnostic tool and a druggable target for the development of cancer immunotherapies of diseases associated with epithelial cell-derived tumors.

It has been reported that tight junction permeability is often higher in tumor tissues than in normal tissues, leading to the speculation that claudin proteins on tumor cells may be more accessible than in normal tissues with intact tight junctions. This possibility makes claudin proteins attractive targets for therapeutic cancer interventions. In addition, published expression profiling results suggest that cancer therapies targeting CLDN18.2 will have favorable systemic toxicity profiles because normal turnover and homeostasis processes replenish gastrointestinal epithelial cells every two to seven days (Sahin et al). Transient gastrointestinal toxicity of limited duration is a common and manageable adverse event for cancer immunotherapeutics.

Pancreatic and gastroesophageal cancers are among the malignancies with the highest unmet medical need (Sahin, et al). Despite the fact that gastric cancer and pancreatic cancer contribute to significant cancer-related morbidity and mortality, the treatment options are limited. Thus, the need exists for anti-CLDN18.2 specific antibodies and binding agents for use in the immunotherapy of cancer associated with epithelial cell-derived primary and metastatic solid tumors.

CLDN18.2 comprises four membrane spanning domains with two small extracellular loops (loop 1 embraced by hydrophobic region 1 and hydrophobic region 2; loop 2 embraced by hydrophobic regions 3 and 4). CLDN18.2 is a transmembrane protein, therefore epitopes present within, or formed by, its extracellular loops represent desirable targets for antibody-based cancer immunotherapy. However, given that CLDN18.1 is expressed by alveolar epithelial cells in normal lung tissue, which is a tissue that is highly relevant to toxicity, exclusive splice variant specificity was a recognized prerequisite for the use of CLDN18.2-specific antibodies for antibody-based cancer immunotherapy. Sahin et al were the first to report proof-of-concept results validating CLDN18.2 as a druggable target for cancer immunotherapies based on the isolation of antibodies (polyclonal and monoclonal) that exclusively bind to CLDN18.2 and not to CLDN18.1 (Sahin et al, Clin. Cancer Res. 14 (23) 7624-7634, 2008).

CLDN18.2 is expressed in a number of primary tumors and their metastasis, including gastric cancer, esophageal cancer, pancreatic cancer, lung cancer such as non-small cell lung cancer, ovarian cancer, colon cancer, hepatic cancer, head-neck cancer, and cancers of the gall bladder. Dysregulated expression of claudins are detected in many cancers and may contribute to tumorigenesis and cancer invasiveness (Singh et al, J Oncology 2010; 2010:541957). The expression of CLDN18.2 is notably elevated in pancreatic ductal adenocarcinomas (PDAC) (Tanaka et al, J Histochem Cytochem. 2011; 59:942-952), esophageal tumors, non-small cell lung cancers (NSCLC), ovarian cancers (Sahin et al., Hu Cancer Biol. 2008; 14:7624-7634), and bile duct adenocarcinomas (Keira et al, Virchows Arch. 2015; 466:265-277).

Despite the fact that gastric cancer contributes to significant cancer-related morbidity and mortality, the treatment options for gastric cancer are limited. Claudins are present in normal tissues, benign neoplasms, hyperplastic conditions and cancers (Ding et al., Cancer Manag. Res. 5:367-375 (2013)). The expression pattern of claudins is highly tissue-specific, and most tissues express multiple claudins. Claudin proteins can interact with claudins from adjacent cells in a homotypic or heterotypic fashion to form tight junctions (Ding et al.). Alterations in claudin expression and signaling pathways are known to be associated with cancer development and an association between the function of impaired tight junctions and tumor progression has been widely reported.

### Nectin Protein Family

Nectins (from the Latin word “necto” meaning “to connect”) interact with Nectins on other cell surface molecules through their Ig-like V-domain of their ECD. Nectins first bind to form cis-dimers on the same cell, and then function to promote cell-cell adhesion by forming homophilic or heterophilic trans-dimers with Nectins or other members of the immunoglobulin super family (IgSF) on an adjacent cell (Miyoshi et al., Am J Nephrol, 27:590, 2007). Heterophilic trans-dimers have been reported to form stronger cell-cell interactions than homophilic trans-dimers. The specificity of binding is different for each Nectin (e.g., Nectin-4 binds to itself and to Nectin-1).

The human Nectin family comprises 9 homologues (Nectin-1 to Nectin-4 and Nectin-like-1 to -5) (Duraivelan et al., Sci Rep, 10:9434, 2020). Nectin proteins (Nectin-1, Nectin-2, Nectin-3, and Nectin-4), are calcium-independent immunoglobulin super family (IgSF) cell adhesion molecules that homophilically or heterophilically trans-interact to mediate cell-cell adhesion at adherens junctions in epithelial cells. In normal epithelium, adherens junctions define cell polarity, a characteristic that is often lost during tumorigenesis.

Nectin-1, -2, -3, and -4 are expressed as single-pass type I glycoproteins, and are characterized by a common domain organization, consisting of an extracellular domain (ECD) with three tandem immunoglobulin-like domains/loops arranged as an N-terminal Ig-like variable domain (D1) followed by two Ig-like constant domains (D2 and D3). Nectins interact with each other via V-domain to V-domain binding interactions thereby creating a trans-hetero-interaction network supporting cell-cell adhesion. Heterophilic interactions among Nectin-3/Nectin-1, Nectin-3/Nectin-2, Nectin-1/Nectin-4 have been reported (Harrison et al., Nat Struct Mol Biol, 19 (9): 906-915, 2012). In addition to their role in cell-cell adhesion the Nectins play important roles in regulating a diverse range of physiologic cellular activities, in viral entry and in immune modulation.

The members of the Nectin family are expressed as single-pass type I glycoproteins, and are characterized by a common domain organization, consisting of three Ig-like domains in the ectodomain (membrane distal IgV domain followed by two IgC domains) a transmembrane region and a cytoplasmic domain (Samanta et al., Cell Mol Life Sci, 72 (4): 645-658, 2015) that binds to the actin cytoskeleton through the adaptor protein afadin.

Many viruses exploit IgSF member proteins to facilitate viral tropism, attachment and subsequent entry into host cells. Several members of the Nectin family were identified as viral receptors before finding their physiological functions as cell adhesion molecules. Initially, members of the Nectin family were independently identified by multiple groups as viral entry receptors and assigned names based on the observed functions. Nectin-1, -2 and -3 were originally described as molecules homologous to the poliovirus receptor (PVR, necl-5, CD155) and as a consequence named Poliovirus Receptor Related (PRR) proteins (nectin1/PRR1/CD111, nectin2/PRR2/CD112 and nectin3/PRR3) (Reymond et al., J Biol Chem, 276 (46): 43205-15, 2001), and subsequently assigned the designations CD111, CD112 and CD113, respectively. Nectin-4 was subsequently demonstrated to recognize the measles virus hemagglutinin (MV-H) and serves as an epithelial cell receptor for measles virus entry (Samanta et al., Cell Mol Life Sci, 72 (4): 645-658, 2015).

Nectins function as cell adhesion molecules by first forming homo cis-dimers on the cell surface and then trans-dimers on adjacent cells in both a homophilic and heterophilic manner. The specificity of binding is different for each Nectin. Nectin-4 binds to itself and Nectin-1 (Reymond et al., J Biol Chem, 276 (46): 43205-15, 2001, Fabre et al., J Biol Chem, 277 (30): 27006-27013, 2002). Cell-cell contacts are thought to be initiated by an interaction between Nectins on adjacent cells. Subsequently, the cadherin-catenin complex is recruited to sites of Nectin-based intercellular adhesion and the trans-interaction of cadherins on adjacent cells occurs, thereby forming the adherens junction (Boylan et al., Oncotarget, 8 (6): 9717-9738, 2017).

The ectodomains of the Nectin proteins share between 30 and 55% amino acid sequence identity. Nectins are connected to the actin cytoskeleton afadin (an F-actin-binding protein) through a binding motif in their cytoplasmic domain, and participate in the organization of epithelial and endothelial junctions. In a complex interplay with other cell adhesion molecules (CAMs) and signal transduction molecules regulate several diverse physiological cellular activities such as movement, proliferation, survival, differentiation, polarization, and the entry of viruses.

The ability of Nectin family members to interact with additional cell surface molecules in mammals significantly expands their interaction network. Nectins are known to cis-interact with other cell surface membrane receptors, such as the platelet-derived growth factor receptor, the fibroblast growth factor receptor, the vascular endothelial growth factor receptor, the prolactin receptor, ErbB2, ErbB3, and ErbB4, and integrins, such as integrin αvβ3 and integrin α6β4, and regulate not only cell-cell adhesion but also cell migration, proliferation, differentiation, and survival (Kedashiro et al., Sci Rep, 9:18997, 2019).

Several members of the Nectin family can exert immunoregulatory functions as a consequence of a heterophilic trans-interaction with another member of the Immunoglobulin superfamily. These interactions are known to impact the functions of diverse immune cell types including natural killer (NK) cells, monocytes, dendritic cells (DCs), and T lymphocytes. Not only are several of the known nectin family interactors IgSF members, some Nectins are known to recognize common binding partners. For example, Nectin-2 and PVR both recognize CD226, TIGIT, and Nectin-3 (Duraivelan et al., Sci Rep, 10:9434, 2020).

A bioinformatics analysis using an algorithm to classify proteins into functionally related families predicted that five additional IgSF members, CD96 (TACTILE), CD226 (DNAM-1), TIGIT (WUCAM, VSTM3), CRTAM, and CD200 were functionally and evolutionarily related to Nectin and Nectin-like proteins and could represent binding partners for members of the Nectin family (Rubinstein et al., Structure, 21 (5): 766-776, 2013). To date, with the exception of CD200, all of these proteins have been reported to bind members of the Nectin-/Nectin-like family (Rubenstein, et al).

The ability of Nectin family members to interact with additional cell surface molecules significantly expands their interaction network. Several members of the Nectin family can exert immunoregulatory functions as a consequence of their heterophilic trans-interaction with another member of the IgSF. These interactions are known to impact the functions of diverse immune cell types including natural killer (NK) cells, monocytes, dendritic cells (DCs), and T lymphocytes. Not only are several of the known Nectin family partners IgSF members, some Nectins are known to recognize common binding partners. For example, Nectin-2 and PVR both recognize CD226, TIGIT and Nectin-3 (Duraivelan et al., Sci Rep, 10:9434, 2020).

### Nectin-4

Nectin-4 (also known as poliovirus-receptor-like 4, PVRL4) was first identified through a bioinformatics search using sequences from known nectin protein ectodomains to identify related sequences (Reymond et al., J Biol Chem, 276 (46): 43205-15, 2001). Human Nectin-4 was cloned from human trachea and described as an antigen with a restricted pattern of expression in normal human tissues. More specifically, it was described as an afadin-associated member of the nectin family that trans-interacts with Nectin-1, but not Nectin-2, Nectin-3, or PVR through a V-domain interaction (Reymond et al., J Biol Chem, 276 (46): 43205-15, 2001).

Reymond and colleagues identified Nectin-4 as a novel ligand for Nectin-1 (Reymond et al., J Biol Chem, 276 (46): 43205-15, 2001), based on their findings that: i) a soluble chimeric recombinant Nectin-4 ectodomain (Nectin-4-Fc) interacts with cells expressing Nectin-1 but not with cells expressing PVR/CD155, Nectin-2, or Nectin-3, and conversely Nectin-1Fc binds to cells expressing Nectin-4; ii) Nectin-1-Fc precipitates Nectin-4 expressed in COS cells and iii) reciprocal in vitro physical interactions were observed between Nectin-4-Fc and Nectin-1-Fc soluble recombinant proteins (Reymond, N et al.). A Nectin-4-Fc/Nectin-4-Fc interaction was also detected indicating that Nectin-4 possesses both homophilic and heterophilic properties.

The human Nectin-4 gene contains nine exons encoding the Nectin-4 adhesion receptor, a 55.5 kDa protein containing 510 amino acids. According to the protein knowledge database UniProtKb, Nectin-4 (Q96NY8) contains an N-terminal signal peptide (1-31 amino acids), an extracellular domain (32-349 amino acids) having three immunoglobulin-like sub-domains (V-type1 32-144 amino acids, C2-typel 148-237 amino acids, C2-type2 248-331 amino acids), a transmembrane domain (350-370 amino acids) and a cytoplasmic domain (371-510amino acids).

It has been reported that the V-like domain of Nectin-4 is sufficient to mediate its trans-interaction with Nectin-1, and that the membrane proximal Nectin-4 C-like domains contribute to increasing the affinity of the trans-interaction (Fabre et al., J Biol Chem, 277 (30): 27006-27013, 2002). Nectin-4 and Nectin-3 share a common binding region in the Nectin-1 V-like domain (Harrison et al., Nat Struct Mol Biol, 19 (9): 906-915, 2012).

It has also been reported that Nectin-4/Nectin-1 trans-interaction is blocked by an anti-Nectin-1 monoclonal antibody (R1.302) whose epitope is localized to the V-like domain of Nectin-1 (Reymond et al., J Biol Chem, 276 (46): 43205-15, 2001). Subsequent publications establish that a monoclonal antibody specific for the Ig-like V domain of Nectin-4 blocks the adhesion of an ovarian cancer cell line engineered to overexpress human Nectin-4 (NIH: OVCAR5) to Nectin-1 (Boylan et al., Oncotarget, 8 (6): 9717-9738, 2017).

Nectin-4 has been reported to be upregulated in various epithelial cell cancers, such as breast cancer (Fabre-Lafay et al., BMC Cancer, 7:73, 2007), lung cancer (Takano et al., Cancer Res, 69 (16): 6694-03, 2009, ovarian cancer (Derycke et al., Am J Clin Pathol, 5:835-845, 2010, pancreatic cancer (Nishiwada et al., J Exp Clin Cancer Res, 34 (1): 30, 2015, gallbladder cancer (Zhang et al., Cancer Lett, 375:179-189, 2016), and gastric cancer (Zhang et al., Hum Pathol, 72:107-116, 2018). These cancers frequently have copy number gains or focal amplifications of the Nectin-4 locus (Pavlova et al., Elife, 2: e00358, 2013).

Recently, evidence has accumulated, showing that Nectins contribute to tumorigenesis and functions to promote metastasis. In particular, Nectin-4 has been implicated in cancer cell adhesion, migration, proliferation and epithelial-mesenchymal transition. In breast cancer, pancreatic cancer and lung cancer, overexpression of Nectin-4, or detection or soluble Nectin-4 in patient serum has been reported to be associated with tumor progression and and/or poor survival (Fabre-Lafay et al., BMC Cancer, 7:73, 2007, Takano et al., Cancer Res, 69 (16): 6694-03, 2009, Derycke et al., Am J Clin Pathol, 5:835-845, 2010, Nishiwada et al., J Exp Clin Cancer Res, 34 (1): 30, 2015, and Lattanzio et al., Oncogenesis, 3: e118, 2014).

### Targeting Tumor Associated Antigens for Cancer Immunotherapy

In the last few years, more evidence has established that tight junctions play a role in cancer cell proliferation, transformation and metastasis. Dysregulation of claudins leads to disruption of tight junctions in epithelial cells which in turn results in loss of cell polarity and impairment of the epithelial integrity. The overexpression of Claudin 6 and/or Claudin 18.2 by tumor cells may be linked to dysregulated localization of claudins as a consequence of the dedifferentiation of tumor cells, or the requirement of rapidly growing cancerous tissues to efficiently absorb nutrients within a tumor mass with abnormal vascularization (Morin P J., Cancer Res. 1; 65 (21): 9603-6, 2005). Decreased cell-cell adhesion and increased mobility of cancer cells are suggested to be the main events of epithelial to mesenchymal transition (EMT), an important step in cancer progression and metastasis.

Nectin-4 was identified as a potential target using suppression subtractive hybridization due to its high level of mRNA expression in bladder cancer (Challita-Eid et al., Cancer Res, 76 (10): 3003-13, 2016). Nectin-4 was originally described as a tumor-specific antigen (TSA) because of early publications reporting restricted expression of Nectin-4 by endothelial cells in the human placenta (Reymond et al., J Biol Chem, 276 (46): 43205-15, 2001), a lack of expression in normal adult tissues, and re-expression in various cancer tissue including breast, ovarian, pancreatic and lung cancers (Fabre-Lafay et al., BMC Cancer, 7:73, 2007, Takano et al., Cancer Res, 69 (16): 6694-03, 2009, Derycke et al., Am J Clin Pathol, 5:835-845, 2010, Pavlova et al., Elife, 2: e00358, 2013, Nishiwada et al., J Exp Clin Cancer Res, 34 (1): 30, 2015, Challita-Eid et al., Cancer Res, 76 (10): 3003-13, 2016).

Immunohistochemical (IHC) study results using a murine antibody (M22-244b3) directed against the extracellular domain of human Nectin-4 and a panel of normal human tissue specimens (representing 36 human organs) demonstrated broader expression in normal tissues in low to moderate levels than was previously reported (Challita-Eid et al.) and identified normal tissues that may have an increased risk of eliciting on target anti-Nectin-4 toxicities. Low levels of weak to moderate homogeneous staining have been reported in human skin keratinocytes, skin appendages (sweat glands and hair follicles, and the epithelia of bladder, stomach, breast, esophagus, and salivary gland (ducts) (Challita-Eid et al., Reymond et al., J Biol Chem, 276 (46): 43205-15, 2001, Brancati et al., Am J Hum Gen, 87:265-273, 2010), suggesting that Nectin-4 is more of a tumor-associated antigen (TAA) than a TSA.

Nectin-4 is overexpressed in multiple cancers, particularly urothelial, lung, pancreatic, breast and ovarian cancer (Challita-Eid et al., Cancer Res, 76 (10): 3003-13, 2016, Fabre-Lafay et al., BMC Cancer, 7:73, 2007, Takano et al., Cancer Res, 69 (16): 6694-03, 2009, Derycke et al., Am J Clin Pathol, 5:835-845, 2010). Extensive immunohistochemical of Nectin-4 expression in a human cancer tumor microarray (TMA) representing 34 tumors representing 7 different indications (e.g., bladder, breast, pancreatic, lung, ovarian, head/neck, and esophageal cancers) established that across evaluated cancer indications, 69% of TMA specimens were positive for Nectin-4. The highest frequencies for overall expression of Nectin-4 were observed for bladder, breast and pancreatic tumors. In the ovarian, lung, head/neck and esophageal cancer samples, the prevalence of Nectin-4-positive samples with moderate to strong staining was generally lower (Chalittta-Eid et al.). The higher Nectin-4 expression levels observed in cancer, theoretically provides a therapeutic window characterized by an acceptable safety profile for anti-Nectin-4 targeted ADCs and antibody-based immunotherapies (Challita-Eid et al., Cancer Res, 76 (10): 3003-13, 2016, and Shim et al., Biomolecules, 10 (3): 360, 2020).

Early stages of epithelial cancer progression are characterized by genetic changes that confer ability to survive and proliferate in the absence of extracellular matrix anchorage. The ability of cancer cells to tolerate the loss of anchorage is critical for the survival of cancer cells and for the pathologic progression of tumorigenesis (e.g., invasion of the underlying stroma, extravasation into blood vessels and metastatic outgrowth as a distal site) (Pavlova et al., Elife, 2: e00358, 2013). Nectin-4 was identified in a gain of function screen for genes that enable cell proliferation independent of matrix anchorage in TL-HMECs (hTERT-immortalized human mammary epithelial cells transduced with SV40 Large T antigen) (Pavlova et al., Elife, 2: e00358, 2013).

Pavlova et al. further reported that Nectin-4 drives the rapid association of TL-HMECs into multicellular clusters in suspension and that antibodies directed to the extracellular domain of Nectin-4 can be used to disrupt the observed cluster formation. Cell clustering was completely abrogated in the presence of anti-Nectin-4 antibodies. Similarly, an antibody targeting the extracellular region of Nectin-1 also inhibited Nectin-4-induced cell clustering.

Pavlova et al. further demonstrated that Nectin-4 promotes clustering of tumor cells with each other by engaging Nectin-1 receptors on adjacent cells, an interaction which triggers integrin β4/SHP-2/c-Src activation in a matrix attachment independent manner. Pavlova et al. proposed a model in which tumor-specific cell-cell contacts and signaling via Nectin-4/Nectin-1 interactions provides a surrogate for cell-matrix signaling and confers a survival advantage that enables anoikis (i.e., induction of apoptosis in cells upon loss of attachment to the extracellular matrix (ECM) and neighboring cells) evasion.

The results of a study conducted to determine the biological significance of Nectin-4 in cellular functions underlying ovarian cancer progression (i.e., cell adhesion, spheroid formation, migration and proliferation) report in vitro data demonstrating that a mAb against the IgV-like domain of Nectin-4 almost completely blocked ovarian cancer cell adhesion to Nectin-1 (Boylan et al., Oncotarget, 8 (6): 9717-9738, 2017). Boylan et al. note that Pavlova used the same anti-Nectin-4 antibody in a mouse xenograft model of breast cancer and observed disruption of tumor cell adhesion and reduced tumor growth in vivo compared to tumors treated with control IgG and based on the combined results speculate that blocking Nectin-4 cell adhesion may be an important component of therapeutic efficacy of anti-Nectin-4 antibodies used for cancer immunotherapy (Boylan et al.).

Publications reporting the results of preclinical studies evaluating the use of anti-Nectin-4 ADCs as monotherapy for the treatment of Nectin-4 expressing tumors validated the clinical development of anti-Nectin-4 antibody-based immunotherapeutics. For example, AGS-22M6E ADC monotherapy was reported to inhibit the growth of tumors in four mouse xenograft models of human bladder, pancreatic, breast and lung cancer. A subsequent publication by M-Rabet et al. confirmed Nectin-4 as a therapeutic target for primary and metastatic triple negative breast cancer (TNBC) based on the observation that an ADC (N41 mAb-vcMMAE) (WO 2017/042210) prepared using a different anti-Nectin-4 antibody induced complete and durable responses in vitro and in vivo in three models of TNBC developed in immunocompromised NSG mice, against primary tumors, metastatic lesions, and local relapses (M-Rabet et al., Annals of Oncology, 28 (4): 769-776, 2017).

Bispecific Binding Proteins that Bind CD137 and a Tumor Associated Antigen

The present disclosure provides bispecific binding proteins that bind CD137 and a tumor associated antigen (TAA) or a tumor specific antigen and fragments thereof. For example, the tumor specific antigen may be any antigen that is expressed on the surface of a tumor cell in a higher amount than on non-tumor cells. In some embodiment, the tumor associated antigen may be Claudin 6, Claudin 18.2, or Nectin-4.

The bispecific binding protein that binds a TAA and CD137 may comprise (a) an antibody scaffold module comprising a first antigen-binding site that binds the TAA and a second antigen-binding site that binds the TAA; and (b) at least one first binding module comprising a third antigen-binding site that binds CD137.

In some embodiments, the bispecific binding protein that binds a tumor associated antigen and CD137 comprises: (a) an antibody scaffold module comprising a means for binding the tumor associated antigen via a first antigen-binding site and a second antigen-binding site; and (b) at least one first binding module comprising a means for binding CD137 via a third antigen-binding site.

In some embodiments, the antibody scaffold module is a Y-shaped antibody having two heavy chains and two light chains. In a further embodiment, the antibody scaffold module is an IgG including for example an IgG1, IgG2, IgG3, or lgG4. In some embodiments, the first binding module is an antibody fragment such as an scFv. In a further embodiment, the scFv is stabilized by the introduction of a disulfide bond.

In some embodiments, the first binding module binds CD137 and has agonistic activity.

In some embodiments, the antibody scaffold module is a bivalent monoclonal antibody. In some embodiments, the antibody scaffold module is a full-length antibody. In some embodiments, the antibody scaffold module is a murine or human antibody. In other embodiments, the antibody scaffold module is a chimeric, bispecific, or humanized antibody. In some embodiments, the antibody scaffold module is symmetric (e.g., a homodimer) or asymmetric (e.g., a heterodimer).

In other embodiments, the antibody scaffold module is an antibody fragment including, for example, an antibody fragment selected from the group consisting of Fab, Fab′, F(ab)₂, Fv, domain antibodies (dAbs), diabodies, triabodies, tetrabodies, miniantibodies, and single-chain antibodies (scFv). The antibody scaffold module may be a chimeric antibody or a bispecific antibody. In alternative embodiments, the antibody scaffold module may be a polypeptide(s) that contain at least a portion of an antibody that is sufficient to confer TAA selective binding to the polypeptide. is a human antibody.

In some embodiments, the antibody scaffold module comprises two heavy chain sequences both having a C-terminus and a N-terminus and two light chain sequences both having a C-terminus and a N-terminus. In some embodiments, the first binding module is covalently attached the C-terminus of one or both of the antibody scaffold module heavy chain sequences, the C-terminus of one or both of the antibody scaffold module light chain sequences, the N-terminus of one or both of the antibody scaffold module heavy chain sequences, the N-terminus of one or both of the antibody scaffold module light chain sequences, or combinations thereof. In a further embodiment, the first binding module that binds CD137 and the antibody scaffold module that binds a TAA are covalently attached to each other directly or through an interlinker. In an embodiment, the first binding module may be human or humanized.

The antibody scaffold module and first binding module may be directly conjugated (e.g., fused) or indirectly conjugated by a linker. Exemplary linkers include glycine-serine linkers including for example, 3×G4S linkers (e.g., GGGGSGGGGSGGGGS (SEQ ID NO: 64)) and 4×G4S linkers (e.g., GGGGSGGGGSGGGGSGGGGS (SEQ ID NO: 65)).

In various embodiments, the bispecific binding proteins provided herein may comprise an antibody scaffold module having substitutions or modifications of the constant region (i.e. the Fc region), including without limitation, amino acid residue substitutions, mutations and/or modifications, which result in a compound with preferred characteristics including, but not limited to: altered pharmacokinetics, increased serum half-life, increase binding affinity, reduced immunogenicity, increased production, altered Fc ligand binding to an Fc receptor (FcR), enhanced or reduced ADCC, CDC, ADCP, TDCC, altered glycosylation and/or disulfide bonds and modified binding specificity.

Several publications report the successful use of protein engineering strategies to design variant human IgG1 Fc domain (CH regions) with optimized FcgR binding profiles and activating/inhibiting (A: I) ratios suitable to optimize cell-mediated effector functions. In particular efforts have focused on increasing the affinity of the Fc domain for the low affinity receptor FcγIIIa. A number of mutations within the Fc domain have been identified that either directly or indirectly enhance binding of Fc receptors and as a result significantly enhance cellular cytotoxicity (Lazar, G. A. PNAS 103:4005-4010 (2006), Shields, R. L. et al, J. Biol. Chem. 276:6591-6604 (2001) Stewart, R. et al., Protein Engineering Design and Selection 24:671-678 (2011) (Richards, J. O. et al, Mol. Cancer Ther. 7:2517-2575 (2008).

The antibody scaffold module may comprise a Fc region (e.g., two antibody heavy chain constant regions). In some embodiments, the Fc region comprises at least one Fc silencing mutation including, for example, L234A L235A or N297A. In some embodiments, the Fc region may comprise one heavy chain constant region having a knobs-in-holes (KiH) mutation to promote dimerization of the heavy chains. Exemplary Fc constant regions for use in the antibody scaffold modules disclosed herein are set forth in SEQ ID NO: 66, SEQ ID NO: 67, SEQ ID NO: 68, SEQ ID NO: 69, and SEQ ID NO: 73. Exemplary constant regions for the light chains of the antibody scaffold modules disclosed herein are set forth in SEQ ID NO: 70 and SEQ ID NO: 71.

In an embodiment, the first binding module that binds CD137 includes CDRs derived from an anti-CD137 antibody or fragment thereof. For example, the first binding module may comprise a VH having a set of CDRs (HCDR1, HCDR2, and HCDR3) disclosed in Table 1. In an embodiment, the first binding module comprises the heavy chain HCDRs of an antibody that binds CD137 including, for example, an antibody comprising the variable heavy domain as set forth in SEQ ID NO: 23.

TABLE 1
First Binding Module VH CDR Sequences
Anti-CD137 Ab

| HCDR1 | HCDR2 | HCDR3 |
|---|---|---|
| SEQ ID NO: 39 | SEQ ID NO: 40 | SEQ ID NO: 41 |

In an embodiment, the first binding module that binds CD137 includes CDRs derived from an anti-CD137 antibody or fragment thereof. For example, the first binding module may comprise a VL having a set of CDRs (LCDR1, LCDR2, and LCDR3) disclosed in Table 1. In an embodiment, the first binding module comprises the LCDRs of an antibody that binds CD137 including, for example, an antibody comprising the variable light domain as set forth in SEQ ID NO: 24.

TABLE 2
First Binding Module VL CDR Sequences
Anti-CD137 Ab

| LCDR1 | LCDR2 | LCDR3 |
|---|---|---|
| SEQ ID NO: 42 | SEQ ID NO: 43 | SEQ ID NO: 44 |

In an embodiment, the first binding module comprises a combination of a VH and a VL having a set of complementarity-determining regions (CDR1, CDR2 and CDR3) selected from the group consisting of:
(i) VH: CDR1: SEQ ID NO: 39, CDR2: SEQ ID NO: 40, CDR3: SEQ ID NO: 41, VL: CDR1: SEQ ID NO: 42, CDR2: SEQ ID NO: 43, CDR3: SEQ ID NO: 44.

In an embodiment, the first binding module that binds CD137 includes CDRs derived from an anti-CD137 antibody or fragment thereof. For example, the first binding module may comprise a VH having a set of CDRs (HCDR1, HCDR2, and HCDR3) disclosed in Table 1 and a VL having a set of CDRs (LCDR1, LCDR2, and LCDR3) disclosed in Table 2.

In another embodiment, the first binding module comprises a VH having an amino acid sequence as set forth in SEQ ID NO: 23. In another embodiment, the first binding module comprises a VL having an amino acid sequence as set forth in SEQ ID NO: 24. In yet another embodiment, the first binding module comprises a VH having an amino acid sequence as set forth in SEQ ID NO: 23; and a VL having an amino acid sequence as set forth in SEQ ID NO: 24.

In some embodiments, the bispecific binding proteins comprise the first binding module that binds CD137 having a KD of 1-10 nM or lower. The binding association constant ka is at 1-10×10⁶ (1/Ms). The binding association constant kd is at 1-10×10⁻² (1/S).

The antibody scaffold module may comprise a set of CDRs from an antibody specific for a TAA.

In an embodiment, the antibody scaffold module binds Claudin 6 and comprises a VH having a set of CDRs (HCDR1, HCDR2, and HCDR3) disclosed in Table 3. In another embodiment, the antibody scaffold module binds Claudin 6 and comprises a VL having a set of CDRs (HCDR1, HCDR2, and HCDR3) disclosed in Table 4. In yet another embodiment, the antibody scaffold module comprises a VH having a set of CDRs (HCDR1, HCDR2, and HCDR3) disclosed in Table 3; and a VL having a set of CDRs (LCDR1, LCDR2, and LCDR3) disclosed in Table 4.

TABLE 3
Claudin 6 Antibody Scaffold VH CDR Sequences
Anti-Claudin 6 Ab

| HCDR1 | HCDR2 | HCDR3 |
|---|---|---|
| SEQ ID NO: 45 | SEQ ID NO: 46 | SEQ ID NO: 47 |
| SEQ ID NO: 51 | SEQ ID NO: 52 | SEQ ID NO: 53 |

TABLE 4
Claudin 6 Antibody Scaffold VL CDR Sequences
Anti-Claudin 6 Ab

| LCDR1 | LCDR2 | LCDR3 |
|---|---|---|
| SEQ ID NO: 48 | SEQ ID NO: 49 | SEQ ID NO: 50 |
| SEQ ID NO: 54 | SEQ ID NO: 55 | SEQ ID NO: 56 |

In an embodiment, the antibody scaffold module that binds Claudin 6 comprises a combination of a VH and a VL having a set of complementarity-determining regions (CDR1, CDR2 and CDR3) selected from the group consisting of:
(i) VH: CDR1: SEQ ID NO: 45, CDR2: SEQ ID NO: 46, CDR3: SEQ ID NO: 47, VL: CDR1: SEQ ID NO: 48, CDR2: SEQ ID NO: 49, CDR3: SEQ ID NO: 50 and;
ii) VH: CDR1: SEQ ID NO: 51, CDR2: SEQ ID NO: 52, CDR3: SEQ ID NO: 53, VL: CDR1: SEQ ID NO: 54, CDR2: SEQ ID NO: 55, CDR3: SEQ ID NO: 56.

In another embodiment, the antibody scaffold module comprises a VH having an amino acid sequence as set forth in SEQ ID NO: 25 or SEQ ID NO: 27. In another embodiment, the antibody scaffold module comprises a VL having an amino acid sequence as set forth in SEQ ID NO: 26 or SEQ ID NO: 28. In yet another embodiment, the antibody scaffold module comprises a VH having an amino acid sequence as set forth in SEQ ID NO: 25; and a VL having an amino acid sequence as set forth in SEQ ID NO: 26. In yet another embodiment, the antibody scaffold module comprises a VH having an amino acid sequence as set forth in SEQ ID NO: 27; and a VL having an amino acid sequence as set forth in SEQ ID NO: 28.

In an embodiment, the antibody scaffold module binds Claudin 18.2 and comprises a VH having a set of CDRs (HCDR1, HCDR2, and HCDR3) disclosed in Table 5. In another embodiment, the antibody scaffold module binds Claudin 18.2 and comprises a VL having a set of CDRs (HCDR1, HCDR2, and HCDR3) disclosed in Table 6. In yet another embodiment, the antibody scaffold module comprises a VH having a set of CDRs (HCDR1, HCDR2, and HCDR3) disclosed in Table 5; and a VL having a set of CDRs (LCDR1, LCDR2, and LCDR3) disclosed in Table 6.

TABLE 5
Claudin 18.2 Antibody Scaffold VH CDR Sequences
Anti-Claudin 18.2 Ab

| HCDR1 | HCDR2 | HCDR3 |
|---|---|---|
| SEQ ID NO: 33 | SEQ ID NO: 34 | SEQ ID NO: 35 |

TABLE 6
Claudin 18.2 Antibody Scaffold VL CDR Sequences
Anti-Claudin 18.2 Ab

| LCDR1 | LCDR2 | LCDR3 |
|---|---|---|
| SEQ ID NO: 36 | SEQ ID NO: 37 | SEQ ID NO: 38 |

In an embodiment, the antibody scaffold module that binds Claudin 18.2 comprises a combination of a VH and a VL having a set of complementarity-determining regions (CDR1, CDR2 and CDR3) selected from the group consisting of:
(i) VH: CDR1: SEQ ID NO: 33, CDR2: SEQ ID NO: 34, CDR3: SEQ ID NO: 35, VL: CDR1: SEQ ID NO: 36, CDR2: SEQ ID NO: 37, CDR3: SEQ ID NO: 38.

In another embodiment, the antibody scaffold module comprises a VH having an amino acid sequence as set forth in SEQ ID NO: 21. In another embodiment, the antibody scaffold module comprises a VL having an amino acid sequence as set forth in SEQ ID NO: 22. In yet another embodiment, the antibody scaffold module comprises a VH having an amino acid sequence as set forth in SEQ ID NO: 21; and a VL having an amino acid sequence as set forth in SEQ ID NO: 22.

In an embodiment, the antibody scaffold module binds Nectin-4 and comprises a VH having a set of CDRs (HCDR1, HCDR2, and HCDR3) disclosed in Table 7. In another embodiment, the antibody scaffold module binds Nectin-4 and comprises a VL having a set of CDRs (HCDR1, HCDR2, and HCDR3) disclosed in Table 8. In yet another embodiment, the antibody scaffold module comprises a VH having a set of CDRs (HCDR1, HCDR2, and HCDR3) disclosed in Table 7; and a VL having a set of CDRs (LCDR1, LCDR2, and LCDR3) disclosed in Table 8.

TABLE 7
Nectin-4 Antibody Scaffold VH CDR Sequences
Anti-Nectin-4 Ab

| HCDR1 | HCDR2 | HCDR3 |
|---|---|---|
| SEQ ID NO: 57 | SEQ ID NO: 58 | SEQ ID NO: 59 |

TABLE 8
Nectin-4 Antibody Scaffold VL CDR Sequences
Anti-Nectin-4 Ab

| LCDR1 | LCDR2 | LCDR3 |
|---|---|---|
| SEQ ID NO: 60 | SEQ ID NO: 61 | SEQ ID NO: 62 |

In an embodiment, the antibody scaffold module that binds Nectin-4 comprises a combination of a VH and a VL having a set of complementarity-determining regions (CDR1, CDR2 and CDR3) selected from the group consisting of:
(i) VH: CDR1: SEQ ID NO: 57, CDR2: SEQ ID NO: 58, CDR3: SEQ ID NO: 59, VL: CDR1: SEQ ID NO: 60, CDR2: SEQ ID NO: 61, CDR3: SEQ ID NO: 62.

In another embodiment, the antibody scaffold module comprises a VH having an amino acid sequence as set forth in SEQ ID NO: 29 or SEQ ID NO: 31. In another embodiment, the antibody scaffold module comprises a VL having an amino acid sequence as set forth in SEQ ID NO: 30 or SEQ ID NO: 32. In yet another embodiment, the antibody scaffold module comprises a VH having an amino acid sequence as set forth in SEQ ID NO: 29; and a VL having an amino acid sequence as set forth in SEQ ID NO: 30. In yet another embodiment, the antibody scaffold module comprises a VH having an amino acid sequence as set forth in SEQ ID NO: 31; and a VL having an amino acid sequence as set forth in SEQ ID NO: 32.

In another embodiment, the antibody scaffold module comprises a pair of variable heavy chain and variable light chain sequences, selected from the following combinations:
i) a variable heavy chain sequence that is 90%, 95%, or 99% identical to SEQ ID NO: 21 and a variable light chain sequence that is 90%, 95%, or 99% identical to SEQ ID NO: 22; and
ii) a variable heavy chain sequence that is 90%, 95%, or 99% identical to SEQ ID NO: 23 and a variable light chain sequence that is 90%, 95%, or 99% identical to SEQ ID NO: 24.
iii) a variable heavy chain sequence that is 90%, 95%, or 99% identical to SEQ ID NO: 25 and a variable light chain sequence that is 90%, 95%, or 99% identical to SEQ ID NO: 26.
iv) a variable heavy chain sequence that is 90%, 95%, or 99% identical to SEQ ID NO: 27 and a variable light chain sequence that is 90%, 95%, or 99% identical to SEQ ID NO: 28.
v) a variable heavy chain sequence that is 90%, 95%, or 99% identical to SEQ ID NO: 29 and a variable light chain sequence that is 90%, 95%, or 99% identical to SEQ ID NO: 30.
vi) a variable heavy chain sequence that is 90%, 95%, or 99% identical to SEQ ID NO: 31 and a variable light chain sequence that is 90%, 95%, or 99% identical to SEQ ID NO: 32. The skilled person will further understand that the variable light and variable heavy chains may be independently selected, or mixed and matched, to prepare an anti-CLDN6 antibody comprising a combination of variable heavy and variable light chain that is distinct from the pairings identified above.

In some embodiments, the bispecific binding proteins comprise one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the bispecific binding protein containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Scand Suppl 643:55-67; Sasaki et al. (1998) Adv Biophys 35:1-24). Amino acid substitutions to the bispecific binding proteins of the disclosure may be made by known methods for example by PCR mutagenesis (U.S. Pat. No. 4,683,195).

In some embodiments, the first binding module that binds to CD137 comprises a variable heavy chain sequence that comprises an amino acid sequence with at least about 95%, about 96%, about 97%, about 98%, or about 99%, sequence identity to the amino acid sequence set forth in SEQ ID NO: 23. In other embodiments, the first binding module that binds to CD137 retains the binding and/or functional activity of a binding module that binds to CD137 that comprises the variable heavy chain sequence of SEQ ID No: 23. In still further embodiments, the first binding module that binds CD137 comprises the variable heavy chain sequence of SEQ ID No: 23 and has one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NO: 23 (based on the numbering system of Kabat).

In particular embodiments, the first binding module that binds to CD137 comprises a variable heavy chain sequence with at least about 95%, about 96%, about 97%, about 98%, or about 99% sequence identity to a variable region sequence set forth in SEQ ID NO: 23, comprises one or more conservative amino acid substitutions in a framework region (based on the numbering system of Kabat), and retains the binding and/or functional activity of a first binding module that binds to CD137 and that comprises a variable heavy chain sequence as set forth in SEQ ID NO: 23 and a variable light chain sequence as set forth in SEQ ID NO: 24.

In some embodiments, the first binding module that binds to CD137 comprises a variable light chain sequence that comprises an amino acid sequence with at least about 95%, about 96%, about 97%, about 98%, or about 99%, sequence identity to the amino acid sequence set forth in SEQ ID NO: 24. In other embodiments, the first binding module that binds to CD137 retains the binding and/or functional activity of a binding module that binds to CD137 that comprises the variable light chain sequence of SEQ ID NO: 24. In still further embodiments, the first binding module that binds CD137 comprises the variable light chain sequence of SEQ ID No: 24 and has one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NO: 24 (based on the numbering system of Kabat).

In particular embodiments, the first binding module that binds to CD137 comprises a variable light chain sequence with at least about 95%, about 96%, about 97%, about 98%, or about 99% sequence identity to a variable region sequence set forth in SEQ ID NO: 24, comprises one or more conservative amino acid substitutions in a framework region (based on the numbering system of Kabat), and retains the binding and/or functional activity of a first binding module that comprises a variable heavy chain sequence as set forth in SEQ ID NO: 23 and a variable light chain sequence as set forth in SEQ ID NO: 24.

In some embodiments, the antibody scaffold module that binds to Claudin 6 comprises a variable heavy chain sequence that comprises an amino acid sequence with at least about 95%, about 96%, about 97%, about 98%, or about 99%, sequence identity to the amino acid sequence set forth in SEQ ID NOs: 25 or 27. In other embodiments, the antibody scaffold module that binds to Claudin 6 retains the binding and/or functional activity of a binding module that binds to Claudin 6 that comprises the variable heavy chain sequence of SEQ ID Nos: 25 or 27. In still further embodiments, the antibody scaffold module that binds Claudin 6 comprises the variable heavy chain sequence of SEQ ID Nos: 25 or 27 and has one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 25 or 27 (based on the numbering system of Kabat).

In particular embodiments, the antibody scaffold module that binds to Claudin 6 comprises a variable heavy chain sequence with at least about 95%, about 96%, about 97%, about 98%, or about 99% sequence identity to a variable region sequence set forth in SEQ ID NOs: 25 or 27, comprises one or more conservative amino acid substitutions in a framework region (based on the numbering system of Kabat), and retains the binding and/or functional activity of an antibody scaffold module that binds to Claudin 6 and that comprises a variable heavy chain sequence as set forth in SEQ ID NOs: 25 or 27 and a variable light chain sequence as set forth in SEQ ID NOs: 26 or 28.

In some embodiments, the antibody scaffold module that binds to Claudin 6 comprises a variable light chain sequence that comprises an amino acid sequence with at least about 95%, about 96%, about 97%, about 98%, or about 99%, sequence identity to the amino acid sequence set forth in SEQ ID NOs: 26 or 28. In other embodiments, the antibody scaffold module that binds to Claudin 6 retains the binding and/or functional activity of an antibody scaffold module that binds to Claudin 6 that comprises the variable light chain sequence of SEQ ID Nos: 26 or 28. In still further embodiments, the antibody scaffold module that binds Claudin 6 comprises the variable light chain sequence of SEQ ID Nos: 26 or 28 and has one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 26 or 28 (based on the numbering system of Kabat).

In particular embodiments, the antibody scaffold module that binds to Claudin 6 comprises a variable light chain sequence with at least about 95%, about 96%, about 97%, about 98%, or about 99% sequence identity to a variable region sequence set forth in SEQ ID NOs: 26 or 28, comprises one or more conservative amino acid substitutions in a framework region (based on the numbering system of Kabat), and retains the binding and/or functional activity of an antibody scaffold module that comprises a variable heavy chain sequence as set forth in SEQ ID NOs: 25 or 27 and a variable light chain sequence as set forth in SEQ ID NOs: 26 or 28.

In some embodiments, the antibody scaffold module that binds to Claudin 18.2 comprises a variable heavy chain sequence that comprises an amino acid sequence with at least about 95%, about 96%, about 97%, about 98%, or about 99%, sequence identity to the amino acid sequence set forth in SEQ ID NO: 21. In other embodiments, the antibody scaffold module that binds to Claudin 18.2 retains the binding and/or functional activity of a binding module that binds to Claudin 18.2 that comprises the variable heavy chain sequence of SEQ ID NO: 21. In still further embodiments, the antibody scaffold module that binds Claudin 18.2 comprises the variable heavy chain sequence of SEQ ID NO: 21 and has one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NO: 21 (based on the numbering system of Kabat).

In particular embodiments, the antibody scaffold module that binds to Claudin 18.2 comprises a variable heavy chain sequence with at least about 95%, about 96%, about 97%, about 98%, or about 99% sequence identity to a variable region sequence set forth in SEQ ID NO: 21, comprises one or more conservative amino acid substitutions in a framework region (based on the numbering system of Kabat), and retains the binding and/or functional activity of an antibody scaffold module that binds to Claudin 18.2 and that comprises a variable heavy chain sequence as set forth in SEQ ID NO: 21 and a variable light chain sequence as set forth in SEQ ID NO: 22.

In some embodiments, the antibody scaffold module that binds to Claudin 18.2 comprises a variable light chain sequence that comprises an amino acid sequence with at least about 95%, about 96%, about 97%, about 98%, or about 99%, sequence identity to the amino acid sequence set forth in SEQ ID NO: 22. In other embodiments, the antibody scaffold module that binds to Claudin 18.2 retains the binding and/or functional activity of an antibody scaffold module that binds to Claudin 18.2 that comprises the variable light chain sequence of SEQ ID NO: 22. In still further embodiments, the antibody scaffold module that binds Claudin 18.2 comprises the variable light chain sequence of SEQ ID NO: 22 and has one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NO: 22 (based on the numbering system of Kabat).

In particular embodiments, the antibody scaffold module that binds to Claudin 18.2 comprises a variable light chain sequence with at least about 95%, about 96%, about 97%, about 98%, or about 99% sequence identity to a variable region sequence set forth in SEQ ID NOs: 22, comprises one or more conservative amino acid substitutions in a framework region (based on the numbering system of Kabat), and retains the binding and/or functional activity of an antibody scaffold module that comprises a variable heavy chain sequence as set forth in SEQ ID NO: 21 and a variable light chain sequence as set forth in SEQ ID NO: 22.

In some embodiments, the antibody scaffold module that binds to Nectin-4 comprises a variable heavy chain sequence that comprises an amino acid sequence with at least about 95%, about 96%, about 97%, about 98%, or about 99%, sequence identity to the amino acid sequence set forth in SEQ ID NOs: 29 or 31. In other embodiments, the antibody scaffold module that binds to Nectin-4 retains the binding and/or functional activity of a binding module that binds to Nectin-4 that comprises the variable heavy chain sequence of SEQ ID NOs: 29 or 31. In still further embodiments, the antibody scaffold module that binds Nectin-4 comprises the variable heavy chain sequence of SEQ ID NOs: 29 or 31 and has one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 29 or 31 (based on the numbering system of Kabat).

In particular embodiments, the antibody scaffold module that binds to Nectin-4 comprises a variable heavy chain sequence with at least about 95%, about 96%, about 97%, about 98%, or about 99% sequence identity to a variable region sequence set forth in SEQ ID NOs: 29 or 31, comprises one or more conservative amino acid substitutions in a framework region (based on the numbering system of Kabat), and retains the binding and/or functional activity of an antibody scaffold module that binds to Nectin-4 and that comprises a variable heavy chain sequence as set forth in SEQ ID NOs: 29 or 31 and a variable light chain sequence as set forth in SEQ ID NOs: 30 or 32.

In some embodiments, the antibody scaffold module that binds to Nectin-4 comprises a variable light chain sequence that comprises an amino acid sequence with at least about 95%, about 96%, about 97%, about 98%, or about 99%, sequence identity to the amino acid sequence set forth in SEQ ID NOs: 30 or 32. In other embodiments, the antibody scaffold module that binds to Nectin-4 retains the binding and/or functional activity of an antibody scaffold module that binds to Nectin-4 that comprises the variable light chain sequence of SEQ ID NOs: 30 or 32. In still further embodiments, the antibody scaffold module that binds Nectin-4 comprises the variable light chain sequence of SEQ ID NOs: 30 or 32 and has one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 30 or 32 (based on the numbering system of Kabat).

In particular embodiments, the antibody scaffold module that binds to Nectin-4 comprises a variable light chain sequence with at least about 95%, about 96%, about 97%, about 98%, or about 99% sequence identity to a variable region sequence set forth in SEQ ID NOs: 30 or 32 comprises one or more conservative amino acid substitutions in a framework region (based on the numbering system of Kabat), and retains the binding and/or functional activity of an antibody scaffold module that comprises a variable heavy chain sequence as set forth in SEQ ID NOs: 29 or 31 and a variable light chain sequence as set forth in SEQ ID NOs: 30 or 32.

In some embodiments, a bispecific binding protein comprises: SEQ ID NO: 3 and SEQ ID NO: 2 (1901 Ab2), SEQ ID NO: 4 and SEQ ID NO: 5 (1901 Ab3), SEQ ID NO: 12 and SEQ ID NO: 9 (1912 Ab3), SEQ ID NO: 13 and SEQ ID NO: 11 (1912 Ab4), SEQ ID NO: 72 and SEQ ID NO: 9 (1912 Ab5), SEQ ID NO: 14 and SEQ ID NO: 15 (1925 Ab1), SEQ ID NO: 16 and SEQ ID NO: 17 (1925 Ab2), or SEQ ID NO: 18 and SEQ ID NO: 15 (1925 Ab3).

In other embodiments, a bispecific binding protein comprises SEQ ID NO: 3 and SEQ ID NO: 2 (1901 Ab2) and binds CD137 and Claudin 18.2. In other embodiments, a bispecific binding protein comprises SEQ ID NO: 4 and SEQ ID NO: 5 (1901 Ab3) and binds CD137 and Claudin 18.2. In other embodiments, a bispecific binding protein comprises SEQ ID NO: 12 and SEQ ID NO: 9 (1912 Ab3) and binds CD137 and Claudin 6. In other embodiments, a bispecific binding protein comprises SEQ ID NO: 13 and SEQ ID NO: 11 (1912 Ab4) and binds CD137 and Claudin 6. In other embodiments, a bispecific binding protein comprises SEQ ID NO: 72 and SEQ ID NO: 9 (1912 Ab5) and binds CD137 and Claudin 6. In other embodiments, a bispecific binding protein comprises SEQ ID NO: 14 and SEQ ID NO: 15 (1925 Ab1) and binds CD137 and Nectin-4. In other embodiments, a bispecific binding protein comprises SEQ ID NO: 16 and SEQ ID NO: 17 (1925 Ab2) and binds CD137 and Nectin-4. In other embodiments, a bispecific binding protein comprises SEQ ID NO: 18 and SEQ ID NO: 15 (1925 Ab3) and binds CD137 and Nectin-4.

In an embodiment, a bispecific binding protein that binds CD137 and Claudin 18.2 comprises:
i) an antibody scaffold module that binds to Claudin 18.2, wherein the antibody scaffold module is an IgG having two heavy chains and two light chains, wherein the IgG comprises an Fc region comprising two constant chains having an N- and a C-terminus; and
ii) two first binding modules that bind CD137, wherein the first binding modules are an scFv, wherein the scFv comprises a VH and a VL linked by a 4×(G4S) linker, and wherein each of the first binding modules is separately attached to the C-terminus of the Fc constant chains by a 3×(G4S) linker.

In some embodiments, the 3×(G4S) linker has an N-terminus and a C-terminus, wherein the N-terminus of the 3×(G4S) linker is attached to the C-terminus of the two Fc constant chains and the C-terminus of the 3×(G4S) linker is attached to the N-terminus of the VH in the first binding module. The scFv may be stabilized. In some embodiments, both of the two heavy chains of the antibody scaffold module, the 3×(G4S) linker, and the first binding module comprise an amino acid sequence from N to C-terminus as set forth in SEQ ID NO: 3. In a further embodiment, the antibody scaffold module comprises two light chains each having an amino acid sequence as set forth in SEQ ID NO: 2.

In an embodiment, a bispecific binding protein that binds CD137 and Claudin 18.2 comprises:
i) an antibody scaffold module that binds to Claudin 18.2, wherein the antibody scaffold module is an IgG having two heavy chains and two light chains, wherein the IgG comprises an Fc region comprising two constant chains having an N- and a C-terminus; and
ii) two first binding modules that bind CD137, wherein the first binding modules are an scFv, wherein the scFv comprises a VH and a VL linked by a 4×(G4S) linker, and wherein each of the first binding modules is separately attached to the C-terminus of the light chains by a 3×(G4S) linker.

In some embodiments, the 3×(G4S) linker has an N-terminus and a C-terminus, wherein the N-terminus of the 3× (G4S) linker is attached to the C-terminus of the two light chains and the C-terminus of the 3× (G4S) linker is attached to the N-terminus of the VH in the first binding module. The scFv may be stabilized. In some embodiments, the two light chains of the antibody scaffold module, the 3× (G4S) linker, and the first binding module each separately comprise an amino acid sequence from N to C-terminus as set forth in SEQ ID NO: 5. In further embodiment, the antibody scaffold module comprises two heavy chains each having an amino acid sequence as set forth in SEQ ID NO: 4.

In an embodiment, a bispecific binding protein that binds CD137 and Claudin 6 comprises:
i) an antibody scaffold module that binds to Claudin 6, wherein the antibody scaffold module is an IgG having two heavy chains and two light chains, wherein the IgG comprises an Fc region comprising two constant chains having an N- and a C-terminus; and
ii) two first binding modules that bind CD137, wherein the first binding modules are an scFv, wherein the scFv comprises a VH and a VL linked by a 4× (G4S) linker, and wherein each of the first binding modules is separately attached to the C-terminus of the Fc constant chains by a 3× (G4S) linker.

In some embodiments, the 3× (G4S) linker has an N-terminus and a C-terminus, wherein the N-terminus of the 3× (G4S) linker is attached to the C-terminus of the two Fc constant chains and the C-terminus of the 3× (G4S) linker is attached to the N-terminus of the VH in the first binding module. The scFv may be stabilized. In some embodiments, the two heavy chains of the antibody scaffold module, the 3× (G4S) linker, and the first binding module each separately comprise an amino acid sequence from N to C-terminus as set forth in SEQ ID NO: 12 or SEQ ID NO: 72. In further embodiment, the antibody scaffold module comprises two light chains each having an amino acid sequence as set forth in SEQ ID NO: 9.

In an embodiment, a bispecific binding protein that binds CD137 and Claudin 6 comprises:
i) an antibody scaffold module that binds to Claudin 6, wherein the antibody scaffold module is an IgG having two heavy chains and two light chains, wherein the IgG comprises a Fc region comprising two constant chains having an N- and a C-terminus; and
ii) two first binding modules that bind CD137, wherein the first binding modules are an scFv, wherein the scFv comprises a VH and a VL linked by a 4× (G4S) linker, and wherein each of the first binding modules are separately attached to the C-terminus of the light chains by a 3× (G4S) linker.

In some embodiments, the 3× (G4S) linker has an N-terminus and a C-terminus, wherein the N-terminus of the 3× (G4S) linker is attached to the C-terminus of the two light chains and the C-terminus of the 3× (G4S) linker is attached to the N-terminus of the VH in the first binding module. The scFv may be stabilized. In some embodiments, the two light chains of the antibody scaffold module, the 3× (G4S) linker, and the first binding module each separately comprise an amino acid sequence from N to C-terminus as set forth in SEQ ID NO: 11. In further embodiment, the antibody scaffold module comprises two heavy chains each having an amino acid sequence as set forth in SEQ ID NO: 13.

In an embodiment, a bispecific binding protein that binds CD137 and Nectin-4 comprises:
i) an antibody scaffold module that binds to Nectin-4, wherein the antibody scaffold module is an IgG having two heavy chains and two light chains, wherein the IgG comprises an Fc region comprising two constant chains having an N- and a C-terminus; and
ii) two first binding modules that bind CD137, wherein the first binding modules are an scFv, wherein the scFv comprises a VH and a VL linked by a 4× (G4S) linker, and wherein each of the first binding modules is separately attached to the C-terminus of the Fc constant chains by a 3× (G4S) linker.

In some embodiments, the 3× (G4S) linker has an N-terminus and a C-terminus, wherein the N-terminus of the 3× (G4S) linker is attached to the C-terminus of the two Fc constant chains and the C-terminus of the 3× (G4S) linker is attached to the N-terminus of the VH in the first binding module. The scFv may be stabilized. In some embodiments, the two heavy chains of the antibody scaffold module, the 3× (G4S) linker, and the first binding module each separately comprise an amino acid sequence from N to C-terminus as set forth in SEQ ID NO: 14. In another embodiment, the antibody scaffold module comprises two light chains each having an amino acid sequence as set forth in SEQ ID NO: 15.

In an embodiment, a bispecific binding protein that binds CD137 and Nectin-4 comprises:
i) an antibody scaffold module that binds to Nectin-4, wherein the antibody scaffold module is an IgG having two heavy chains and two light chains, wherein the IgG comprises an Fc region comprising two constant chains having an N- and a C-terminus; and
ii) two first binding modules that bind CD137, wherein the first binding modules are an scFv, wherein the scFv comprises a VH and a VL linked by a 4× (G4S) linker, and wherein each of the first binding modules is separately attached to the C-terminus of the light chains by a 3× (G4S) linker.

In some embodiments, the 3× (G4S) linker has an N-terminus and a C-terminus, wherein the N-terminus of the 3× (G4S) linker is attached to the C-terminus of the two light chains and the C-terminus of the 3× (G4S) linker is attached to the N-terminus of the VH in the first binding module. The scFv may be stabilized. In some embodiments, the two light chains of the antibody scaffold module, the 3× (G4S) linker, and the first binding module each separately comprise an amino acid sequence from N to C-terminus as set forth in SEQ ID NO: 17. In further embodiment, the antibody scaffold module comprises two heavy chains each having an amino acid sequence as set forth in SEQ ID NO: 16.

In an embodiment, a bispecific binding protein that binds CD137 and Nectin-4 comprises:
i) an antibody scaffold module that binds to Nectin-4, wherein the antibody scaffold module is an IgG having two heavy chains and two light chains, wherein the IgG comprises an Fc region comprising two constant chains having an N- and a C-terminus; and
ii) two first binding modules that bind CD137, wherein the first binding modules are an scFv, wherein the scFv comprises a VH and a VL linked by a 4× (G4S) linker, and wherein each of the first binding modules is separately attached to the N-terminus of the two heavy chains by a 4× (G4S) linker.

In some embodiments, the 4× (G4S) linker has a N-terminus and a C-terminus, wherein the C-terminus of the 4× (G4S) linker is attached to the N-terminus of the two heavy chains and the N-terminus of the 4× (G4S) linker is attached to the C-terminus of the VL in the first binding module. The scFv may be stabilized. In some embodiments, the first binding module, the 4× (G4S) linker, and the two heavy chains of the antibody scaffold module each separately comprise an amino acid sequence from N to C-terminus as set forth in SEQ ID NO: 18. In further embodiment, the antibody scaffold module comprises two light chains each having an amino acid sequence from N to C-terminus as set forth in SEQ ID NO: 15.

The therapeutic value of the bispecific binding proteins of the disclosure can be enhanced by conjugation to a cytotoxic drug or agent that improves its effectiveness and potency including, for example, a cytotoxic effector agent such as a radioisotope, a drug, or a cytotoxin.

In some embodiments, the bispecific binding protein disclosed herein exhibits one or more of the following structural and functional characteristics, alone or in combination:
(a) capable of binding to human CD137 and a tumor associated antigens (TAA);
(b) cross-reacts with cynomolgus CD137 and one of the tumor associated antigens (TAA);
(c) disrupts (e.g., reduces or prevents) human CD137L binding to CD137;
(d) exhibits fast on and fast off properties to CD137;
(e) possess TAA-dependent agonistic activity to CD137 signaling;
(f) activates T cells in TAA-dependent manner; and
(g) kills TAA expressing cells by activating CD8 T cells.

In some embodiments, the bispecific binding protein is a Claudin-6/CD137 BsAb exhibiting one or more of the following structural and functional characteristics, alone or in combination:
(a) bivalency for Claudin6 binding;
(b) fast-on/Fast-off CD137 binding kinetics;
(c) enhances Lymphocyte infiltration in tumors;
(d) promotes T cell proliferation/activation in tumors;
(e) protects T cells from exhaustion in tumors;
(f) promotes T cell memory formation from Tumor-experienced T cells;
(g) decreases Treg/CD8 ratio in the tumor microenvironment (TME); and
(h) decreases M2-like macrophages in TME.

### Methods of Producing Monoclonal Antibodies for Use as Scaffold or Binding Modules

Bispecific binding proteins that bind CD137 and a TAA may be made by any method known in the art. For example, a recipient may be immunized with soluble recombinant CD137 protein or a fragment of a CD137 peptide conjugated with a carrier protein thereof. Similarly, a recipient may be immunized with s soluble recombinant TAA protein or a fragment of a tumor-associated antigen peptide conjugated with a carrier protein thereof. Any suitable method of immunization can be used. Such methods can include adjuvants, other immune stimulants, repeat booster immunizations, and the use of one or more immunization routes. CDRs or VH/VLs obtained from antibodies may be used in the antibody scaffold module and/or first binding module.

Any suitable source of human CD137 or TAA can be used as the immunogen for the generation of the non-human or human anti-CD137 and/or TAA antibodies of the compositions and methods disclosed herein.

Different forms of CD137 and/or TAA may be used to elicit an immune response for the identification of a biologically active anti-CD137 or anti-TAA antibodies. Thus, the eliciting CD137 antigen or TAA may be a single epitope, multiple epitopes, or the entire protein alone or in combination with one or more immunogenicity enhancing agents. In some aspects, the eliciting antigen is an isolated soluble full-length protein, or a soluble protein comprising less than the full-length sequence (e.g., immunizing with a peptide comprising the extracellular domains/loops of CD137 or TAA, ECD1 and/or ECD2 alone or in combination). As used herein, the term “portion” refers to the minimal number of amino acids or nucleic acids, as appropriate, to constitute an immunogenic epitope of the antigen of interest. Any genetic vectors suitable for transformation of the cells of interest may be employed, including, but not limited to adenoviral vectors, plasmids, and non-viral vectors, such as cationic lipids.

It is desirable to prepare monoclonal antibodies (mAbs) from various mammalian hosts, such as mice, rodents, primates, humans, etc. Description of techniques for preparing such monoclonal antibodies may be found in, e.g., Sties et al. (eds.) BASIC AND CLINICAL IMMUNOLOGY (4th ed.) Lance Medical Publication, Los Altos, CA, and references cited therein; Harlow and Lane (1988) ANTIBODIES: A LABORATORY MANUAL CSH Press; Goding (1986) MONOCLONAL ANTIBODIES: PRINCIPLES AND PRACTICE (2nd ed.) Academic Press, New York, NY. Typically, spleen cells from an animal immunized with a desired antigen are immortalized, commonly by fusion with a myeloma cell. See Kohler and Milstein (196) Eur. J. Immunol. 6:511-519. Alternative methods of immortalization include transformation with Epstein Barr Virus, oncogene, or retroviruses, or other methods known in the art. See. e.g., Doyle et al. (eds. 1994 and periodic supplements) CELL AND TISSUE CULTURE: LABORATORY PROCEDURES, John Wiley and Sons, New York, NY. Colonies arising from single immortalized cells are screened for the production of antibodies of the desired specificity and affinity for the antigen, and the yield of the monoclonal antibodies produced by such cells may be enhanced by various techniques, including injection into the peritoneal cavity of a vertebrate host. Alternatively, one may isolate DNA sequences which encode a monoclonal antibody or an antigen binding fragment thereof by screening a DNA library from human B cells according, e.g., to the general protocol outlined by Huse et al. (1989) Science 246:1275-1281. Thus, antibodies may be obtained by a variety of techniques familiar to researchers skilled in the art.

Other suitable techniques involve the selection of libraries of antibodies in phage, yeast, virus or similar vector. See e.g., Huse et al. supra; and Ward et al. (1989) Nature 341:544-546. The polypeptides and antibodies disclosed herein may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,9396,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10023; or made in transgenic mice, see Nils Lonberg et al. (1994), Nature 368:856-859; and Mendez et al. (1997) Nature Genetics 15:146-156; TRANSGENIC ANIMALS AND METHODS OF USE (WO 2012/62118), Medarex, Trianni, Abgenix, Ablexis, OminiAb, Harbour and other technologies.

In some embodiments, the ability of the produced antibody to bind to CD137 or a TAA can be assessed using standard binding assays, such as surface plasmon resonance (SPR), FoteBio (BLI), Gator (BLI), ELISA, Western Blot, Immunofluorescence, flow cytometric analysis (FACS) or an internalization assay.

The antibody composition prepared from the hybridoma or host cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

### Polynucleotides, Vectors, and Host Cells

Other embodiments encompass isolated polynucleotides that comprise a sequence(s) encoding a bispecific binding protein as disclosed herein, vectors, and host cells comprising the polynucleotides, and recombinant techniques for the production of the bispecific binding protein. The isolated polynucleotides can encode any desired form of the bispecific binding protein, including its components such as the scaffold module and/or the first binding module.

In an embodiment, the isolated polynucleotide sequence encodes a first binding module that comprises a combination of a VH and a VL having a set of complementarity-determining regions (CDR1, CDR2 and CDR3) selected from VH: CDR1: SEQ ID NO: 39, CDR2: SEQ ID NO: 40, CDR3: SEQ ID NO: 41; and VL: CDR1: SEQ ID NO: 42, CDR2: SEQ ID NO: 43, CDR3: SEQ ID NO: 44.

In an embodiment, the isolated polynucleotide sequence encodes an antibody scaffold module that binds Claudin 6 and comprises a combination of a VH and a VL having a set of complementarity-determining regions (CDR1, CDR2 and CDR3) selected from VH: CDR1: SEQ ID NO: 45, CDR2: SEQ ID NO: 46, CDR3: SEQ ID NO: 47; and VL: CDR1: SEQ ID NO: 48, CDR2: SEQ ID NO: 49, CDR3: SEQ ID NO: 50.

In an embodiment, the isolated polynucleotide sequence encodes an antibody scaffold module that binds Claudin 6 and comprises a combination of a VH and a VL having a set of complementarity-determining regions (CDR1, CDR2 and CDR3) selected from VH: CDR1: SEQ ID NO: 51, CDR2: SEQ ID NO: 52, CDR3: SEQ ID NO: 53; and VL: CDR1: SEQ ID NO: 54, CDR2: SEQ ID NO: 55, CDR3: SEQ ID NO: 56.

In an embodiment, the isolated polynucleotide sequence encodes an antibody scaffold module that binds Claudin 18.2 and comprises a combination of a VH and a VL having a set of complementarity-determining regions (CDR1, CDR2 and CDR3) selected from VH: CDR1: SEQ ID NO: 33, CDR2: SEQ ID NO: 34, CDR3: SEQ ID NO: 35; and VL: CDR1: SEQ ID NO: 36, CDR2: SEQ ID NO: 37, CDR3: SEQ ID NO: 38.

In an embodiment, the isolated polynucleotide sequence encodes an antibody scaffold module that binds Nectin-4 and comprises a combination of a VH and a VL having a set of complementarity-determining regions (CDR1, CDR2 and CDR3) selected from VH: CDR1: SEQ ID NO: 57, CDR2: SEQ ID NO: 58, CDR3: SEQ ID NO: 59; and VL: CDR1: SEQ ID NO: 60, CDR2: SEQ ID NO: 61, CDR3: SEQ ID NO: 62.

In another embodiment, the isolated polynucleotide sequence encodes an antibody scaffold module that comprises a VH having an amino acid sequence as set forth in SEQ ID NO: 29 or SEQ ID NO: 31. In another embodiment, the isolated polynucleotide sequence encodes an antibody scaffold module that comprises a VL having an amino acid sequence as set forth in SEQ ID NO: 30 or SEQ ID NO: 32. In yet another embodiment, the isolated polynucleotide sequence encodes an antibody scaffold module comprising a VH having an amino acid sequence as set forth in SEQ ID NO: 29; and a VL having an amino acid sequence as set forth in SEQ ID NO: 30. In yet another embodiment, the isolated polynucleotide sequence encodes an antibody scaffold module comprises a VH having an amino acid sequence as set forth in SEQ ID NO: 31; and a VL having an amino acid sequence as set forth in SEQ ID NO: 32.

In another embodiment, the isolated polynucleotide sequence encodes an antibody scaffold module that comprises a pair of variable heavy chain and variable light chain sequences, selected from the following combinations:
i) a variable heavy chain sequence that is 90%, 95%, or 99% identical to SEQ ID NO: 21 and a variable light chain sequence that is 90%, 95%, or 99% identical to SEQ ID NO: 22; and
ii) a variable heavy chain sequence that is 90%, 95%, or 99% identical to SEQ ID NO: 23 and a variable light chain sequence that is 90%, 95%, or 99% identical to SEQ ID NO: 24.
iii) a variable heavy chain sequence that is 90%, 95%, or 99% identical to SEQ ID NO: 25 and a variable light chain sequence that is 90%, 95%, or 99% identical to SEQ ID NO: 26.
iv) a variable heavy chain sequence that is 90%, 95%, or 99% identical to SEQ ID NO: 27 and a variable light chain sequence that is 90%, 95%, or 99% identical to SEQ ID NO: 28.
v) a variable heavy chain sequence that is 90%, 95%, or 99% identical to SEQ ID NO: 29 and a variable light chain sequence that is 90%, 95%, or 99% identical to SEQ ID NO: 30.
vi) a variable heavy chain sequence that is 90%, 95%, or 99% identical to SEQ ID NO: 31 and a variable light chain sequence that is 90%, 95%, or 99% identical to SEQ ID NO: 32.

In some embodiments, the isolated polynucleotide sequence encodes a bispecific binding protein that comprises: SEQ ID NO: 3 and SEQ ID NO: 2 (1901 Ab2), SEQ ID NO: 4 and SEQ ID NO: 5 (1901 Ab3), SEQ ID NO: 12 and SEQ ID NO: 9 (1912 Ab3), SEQ ID NO: 13 and SEQ ID NO: 11 (1912 Ab4), SEQ ID NO: 72 and SEQ ID NO: 9 (1912 Ab5), SEQ ID NO: 14 and SEQ ID NO: 15 (1925 Ab1), SEQ ID NO: 16 and SEQ ID NO: 17 (1925 Ab2), or SEQ ID NO: 18 and SEQ ID NO: 15 (1925 Ab3).

In other embodiments, the isolated polynucleotide sequence encodes a bispecific binding protein that comprises SEQ ID NO: 3 and/or SEQ ID NO: 2 (1901 Ab2) and binds CD137 and Claudin 18.2. In other embodiments, the isolated polynucleotide sequence encodes a bispecific binding protein that comprises SEQ ID NO: 4 and/or SEQ ID NO: 5 (1901 Ab3) and binds CD137 and Claudin 18.2. In other embodiments, the isolated polynucleotide sequence encodes a bispecific binding protein that comprises SEQ ID NO: 12 and/or SEQ ID NO: 9 (1912 Ab3) and binds CD137 and Claudin 6. In other embodiments, the isolated polynucleotide sequence encodes a bispecific binding protein that comprises SEQ ID NO: 13 and/or SEQ ID NO: 11 (1912 Ab4) and binds CD137 and Claudin 6. In other embodiments, the isolated polynucleotide sequence encodes a bispecific binding protein that comprises SEQ ID NO: 72 and/or SEQ ID NO: 9 (1912 Ab5) and binds CD137 and Claudin 6. In other embodiments, the isolated polynucleotide sequence encodes a bispecific binding protein that comprises SEQ ID NO: 14 and/or SEQ ID NO: 15 (1925 Ab1) and binds CD137 and Nectin-4. In other embodiments, the isolated polynucleotide sequence encodes a bispecific binding protein that comprises SEQ ID NO: 16 and/or SEQ ID NO: 17 (1925 Ab2) and binds CD137 and Nectin-4. In other embodiments, the isolated polynucleotide sequence encodes a bispecific binding protein that comprises SEQ ID NO: 18 and/or SEQ ID NO: 15 (1925 Ab3) and binds CD137 and Nectin-4.

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode a bispecific binding protein of the present disclosure. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, or at least 98%, identical to the sequence of a portion or all of a nucleic acid encoding a polypeptide chain of the bispecific binding protein (e.g., a heavy chain or light chain variable region of the antibody scaffold module and/or the first binding module), or its complement. Hybridizing nucleic acids of the type described herein can be used, for example, as a cloning probe, a primer, e.g., a PCR primer, or a diagnostic probe.

The polynucleotide(s) that comprise a sequence encoding a bispecific binding protein as disclosed herein can be fused to one or more regulatory or control sequences, as known in the art, and can be contained in suitable expression vectors or cells as known in the art. Each of the polynucleotide molecules encoding the heavy or light chain variable domains of the antibody binding scaffold can be independently fused to a polynucleotide sequence encoding a constant domain, such as a human constant domain to form an antibody scaffold module. Alternatively, polynucleotides, or portions thereof, can be fused together, providing a template for the production of the first binding module.

For recombinant production, a polynucleotide encoding the bispecific binding protein (e.g., its antibody scaffold module including two heavy and two lights chains and first binding module) disclosed herein is inserted into a replicable vector for cloning (amplification of the DNA) or for expression. Many suitable vectors for expressing the bispecific binding protein are available. The vector components generally include, but are not limited to, one or more of the following: a signal sequence, an origin of replication, one or more marker genes, an enhancer element, a promoter, and a transcription termination sequence.

The bispecific binding protein (e.g., its antibody scaffold module including two heavy and two lights chains and the first binding module) can also be produced as fusion polypeptides, in which the bispecific binding protein is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the bispecific binding protein signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in the reading frame to DNA encoding the bispecific binding protein (e.g., its antibody scaffold module including two heavy and two lights chains and the first binding module).

Expression and cloning vectors contain a nucleic acid sequence that enables the vector to replicate in one or more selected host cells. Generally, in cloning vectors this sequence is one that enables the vector to replicate independently of the host chromosomal DNA and includes origins of replication or autonomously replicating sequences. Such sequences are well known for a variety of bacteria, yeast, and viruses. The origin of replication from the plasmid pBR322 is suitable for most Gram-negative bacteria, the 2-v. Plasmid origin is suitable for yeast, and various viral origins (SV40, polyoma, adenovirus, VSV, and BPV) are useful for cloning vectors in mammalian cells. Generally, the origin of the replication component is not needed for mammalian expression vectors (the SV40 origin may typically be used only because it contains the early promoter).

Expression and cloning vectors may contain a gene that encodes a selectable marker to facilitate the identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

Also provided herein are host cells that comprise one or more polynucleotides coding for the bispecific binding protein. The cells used to produce the bispecific binding proteins as disclosed herein may be cultured in a variety of media. Commercially available media such as Ham's F10 (Sigma), Minimal Essential Medium ((MEM), Sigma), RPMI-1640 (Sigma), FreeStyle™ (Gibco) and Dulbecco's Modified Eagle's Medium ((DMEM), Sigma) are suitable for culturing the host cells. Any of these or other media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin), trace elements (such as inorganic compounds usually present at final concentrations in the micromolar or lower range), and glucose or an equivalent energy source. Any other necessary supplements may also be included at appropriate concentrations that would be known to those skilled in the art. The culture conditions, such as temperature, pH, and the like, include those previously used with the cell selected for expression, and will be apparent to those skilled in the art.

### Non-Therapeutic Uses

The bispecific binding proteins described herein are useful as affinity purification agents. In this process, a bispecific binding protein is immobilized on a solid phase such a Protein A resin, using methods well known in the art. The immobilized bispecific binding protein is contacted with a sample containing CD137 and a TAA protein (or a fragment thereof) to be purified, and thereafter the support is washed with a suitable solvent that will remove substantially all the material in the sample except the CD137 and TAA protein, which is bound to the immobilized bispecific binding protein. Finally, the support is washed with another suitable solvent that will release the CD137 and TAA protein from the bispecific binding protein.

The bispecific binding proteins disclosed herein are also useful in diagnostic assays to detect and/or quantify CD137 and/or TAA protein, for example, detecting CD137 and/or TAA expression in specific cells, tissues, or serum. The bispecific binding proteins can be used diagnostically to, for example, monitor the development or progression of a disease as part of a clinical testing procedure to, e.g., determine the efficacy of a given treatment and/or prevention regimen. Detection can be facilitated by coupling the bispecific binding protein to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to bispecific binding proteins for use as diagnostics according to the present disclosure.

The bispecific binding proteins can be used in methods for diagnosing a CD137 and/or TAA-associated disorder (e.g., a disorder characterized by abnormal expression of CD137 and/or TAA) or to determine if a subject has an increased risk of developing a CD137 and/or TAA-associated disorder. Such methods include contacting a biological sample from a subject with a bispecific binding protein disclosed herein and detecting binding of the molecule to CD137 and/or TAA. By “biological sample” is intended any biological sample obtained from an individual, cell line, tissue culture, or other source of cells potentially expressing CD137 and/or TAA. Methods for obtaining tissue biopsies and body fluids from mammals are well known in the art.

In some embodiments, the method can further comprise comparing the level of CD137 and/or TAA in a patient sample to a control sample (e.g., a subject that does not have a CD137 and/or TAA-associated disorder) to determine if the patient has a CD137 and/or TAA-associated disorder or is at risk of developing a CD137 and/or TAA-associated disorder.

It will be advantageous in some embodiments, for example, for diagnostic purposes to label a bispecific binding protein with a detectable moiety. Numerous detectable labels are available, including radioisotopes, fluorescent labels, enzyme substrate labels and the like. The label may be indirectly conjugated with the bispecific binding protein using various known techniques. For example, the bispecific binding protein can be conjugated with biotin and any of the three broad categories of labels mentioned above can be conjugated with avidin, or vice versa. Biotin binds selectively to avidin and thus, the label can be conjugated with the bispecific binding protein in this indirect manner. Alternatively, to achieve indirect conjugation of the label with the bispecific binding protein, the bispecific binding protein can be conjugated with a small hapten (such as digoxin) and one of the different types of labels mentioned above is conjugated with an anti-hapten antibody (e.g., anti-digoxin antibody). Thus, indirect conjugation of the label with the bispecific binding protein can be achieved.

Exemplary radioisotopes labels include ³⁵S, ¹⁴C, ¹²⁵I, ³H, and ¹³¹I. The bispecific binding protein can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available. The fluorescent labels can be conjugated to the bispecific binding protein via known techniques, such as those disclosed in Current Protocols in Immunology, for example. Fluorescence can be quantified using a fluorimeter.

There are various well-characterized enzyme-substrate labels known in the art (see, e.g., U.S. Pat. No. 4,275,149). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to proteinaceous molecules are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic Press, N. Y., 73:147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3,5,5-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

In another embodiment, a bispecific binding protein disclosed herein is used unlabeled and detected with a labeled antibody that binds the bispecific binding protein.

The bispecific binding proteins described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

The bispecific binding protein disclosed herein can be used to inhibit the binding of CD137 and/or TAA to its respective receptor. Such methods comprise administering a bispecific binding protein disclosed herein to a cell (e.g., a mammalian cell) or cellular environment, whereby signaling mediated by the receptor is inhibited. These methods can be performed in vitro or in vivo. By “cellular environment” is intended the tissue, medium, or extracellular matrix surrounding a cell.

### Compositions and Methods of Treatment

The disclosure also provides compositions including, for example, pharmaceutical compositions that comprise a bispecific binding protein as disclosed herein. Such compositions have numerous therapeutic uses for the treatment, prevention, or amelioration of diseases or disorders such as cancer.

Activation of CD137 in TME by TAA-CD137 antibodies can enhance the immune response to cancer cells in patients. Cancers whose growth may be inhibited by the disclosed bispecific antibodies include cancers typically responsive to immunotherapy and those that are not typically responsive to immunotherapy, including immune checkpoint resistant tumors. Cancers can be solid tumors or liquid tumors.

Non limiting examples of the cancers for treatment include bone cancer, skin cancer, uterine cancer, squamous cell carcinoma, small-cell lung cancer (SCLC), non-small cell lung cancer (NSCLC), glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, cervical cancer, stomach cancer, bladder cancer, hepatoma, breast cancer, colon carcinoma, and head and neck cancer, germ cell tumor, melanoma, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the ureter, carcinoma of the renal pelvis, primary CNS lymphoma, spinal axis tumor, brain cancer, brain stem glioma, pituitary adenoma, Kaposi's sarcoma, epidermoid cancer, all types of leukemias, lymphomas, and myelomas, such as acute leukemia (ALL), acute myelogenous leukemia (AML), chronic lymphocytic leukemia (CLL), and chronic myelogenous leukemia (CML), undifferentiated AML (MO), myeloblasts leukemia (MI), lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/TALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC); myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; as well as any combinations of said cancers.

The antibodies described herein may also be used for the treatment of metastatic cancers, unresectable and/or refractory cancers (e.g., cancers refractory to previous immunotherapy), and recurrent cancers. In certain embodiments, a TAA-CD137 Ab is administered to patients having cancer that exhibited an inadequate response to prior treatment, e.g., prior treatment with an Immuno-oncology drug, or patients having cancer that is refractory or resistant, either intrinsically refractory or resistant (e.g., refractory to a PD-1 pathway antagonist) or a wherein the resistance or refractory state is acquired. For example, subjects who are not responsive or not sufficiently responsive to a first therapy or who see disease progression following treatment, e.g., anti-PD-1 treatment, may be treated by administration of a TAA-CD137 antibody alone or in combination with another therapy (e.g., with an anti-PD-1 therapy). In certain embodiments, a TAA-CD137 antibody is administered to patients who have not previously received (i.e., been treated with) an immuno-oncology agent, e.g., a PD-1 pathway antagonist. A TAA-CD137 antibody may be administered with a standard of care treatment. A TAA-CD137 antibody may be administered as maintenance therapy, e.g., a therapy that is intended to prevent the occurrence or recurrence of tumors. An anti-GITR antibody may be administered with another treatment, e.g., radiation, surgery, or chemotherapy.

In humans, some tumors have been shown to be immunogenic such as melanomas. By lowering the threshold of T cell activation via CD137 activation, the tumor responses in the host can be activated, allowing treatment of non-immunogenic tumors or those having limited immunogenicity

In some embodiments, compositions are provided including, for example, pharmaceutical compositions that comprise a bispecific binding protein that binds CD137 and a tumor associated antigen for use as a therapeutic drug for the treatment of patients having cancer. In a particular embodiment, the compositions described herein are administered to cancer patients to kill tumor cells. For example, the compositions described herein can be used to treat a patient with a solid tumor characterized by the presence of cancer cells expressing or overexpressing a tumor associated antigen. In some aspects, the disclosed compositions can be used to treat breast, lung, ovarian, testicular, pancreatic, gastric, gallbladder and urothelial cancer.

The present disclosure also provides methods for the treatment or prevention of cancer comprising administering a composition or formulation that comprises a bispecific binding protein disclosed herein, and optionally another immune-based therapy, to a subject in need thereof.

The disclosed bispecific binding proteins are also useful in methods of treatment of cancer, either alone (e.g., as monotherapies) or in combination with other immunotherapeutic agents and/or a chemotherapy.

The bispecific binding proteins can be administered either alone or in combination with other compositions that are useful for treating an immune-mediated inflammatory disorder or an autoimmune disease.

In some aspects, a composition, e.g., a pharmaceutical composition is provided that comprises one or more bispecific binding proteins disclosed herein. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of the active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the bispecific binding proteins, may be coated in a material to protect the compound from the action of acids and other natural conditions that may inactivate the compound.

A composition can be administered by a variety of methods known in the art. As will be appreciated by the skilled artisan, the route and/or mode of administration will vary depending upon the desired results. The bispecific binding proteins can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

Dosage levels of the bispecific binding proteins in the pharmaceutical compositions may be varied so as to obtain an amount of the bispecific binding proteins which is effective to achieve the desired therapeutic response for a particular subject, composition, and mode of administration, without being toxic to the subject. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions described herein may be administered in effective amounts. An “effective amount” refers to the amount which achieves a desired reaction or the desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease.

In some aspects, the compositions described herein are administered to patients, e.g., in vivo, to treat or prevent a variety of disorders such as those described herein. Preferred patients include human patients having disorders that can be corrected or ameliorated by administering the bispecific binding proteins disclosed herein.

In some aspects, conventional viral and non-viral based gene transfer methods can be used to introduce nucleic acids encoding the bispecific binding proteins, as described herein, in mammalian cells or target tissues. Such methods can be used to administer nucleic acids encoding the bispecific binding proteins to cells in vitro. In some embodiments, the nucleic acids encoding the bispecific binding proteins are administered for in vivo or ex vivo gene therapy uses. In other embodiments, gene delivery techniques are used to study the activity of the bispecific binding proteins in cell based or animal models. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding the bispecific binding proteins include lipofection, microinjection, biolistics, virosomes, liposomes, immunoliposomes, polycation or lipid: nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid: nucleic acid complexes, including targeted liposomes such as immunolipid complexes, is well known to one of skill in the art.

The use of RNA or DNA viral based systems for the delivery of nucleic acids encoding the bispecific binding proteins described herein take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of the bispecific binding proteins the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In one treatment method, pharmaceutical compositions comprising the bispecific binding protein that binds CD137 and a tumor associated antigen can further comprise a therapeutic or toxic agent, either conjugated or unconjugated to the bispecific binding protein that binds CD137 and a tumor associated antigen. In a particular embodiment a bispecific binding protein that binds CD137 and a tumor associated antigen is used to target an ADC with a cytotoxic payload to tumors expressing and/or overexpressing a tumor associated antigen.

The broad scope of this disclosure is best understood with reference to the following examples, which are not intended to limit the disclosures to the specific embodiments. The specific embodiments described herein are offered by way of example only, and the disclosure is to be limited by the terms of the appended claims, along with the full scope of the equivalents to which such claims are entitled.

### EXAMPLES

### General Methods

Methods for protein purification including immunoprecipitation, chromatography, and electrophoresis are described. See, e.g., Coligan et al. (2000) Current Protocols in Protein Science, Vol. 1, John Wiley and Sons, Inc., New York. Chemical analysis, chemical modification, post-translational modification, production of fusion proteins, and glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra.

Hybridoma or cell culture supernatant containing antibody proteins as disclosed herein was purified via HiTrap protein G column (GE, cat. No. 17040401) according to the manufacturer's procedures. Briefly, the supernatant was equilibrated with DPBS (Gibco, cat. No. 14190-136) for 5 CV and loaded via syringe/infusion pump (Legato 200, KDS) at ambient temperature and 3 minute residence time. The column was washed with 5 CV of DPBS and elution was performed with 4 CV of pH 2.8 elution buffer (Fisher Scientific, cat. No. PI21004). Elution was fractionated, and fractions were neutralized with IM Tris-HCL, pH 8.5 (Fisher Scientific, cat No. 50-843-270) and assayed by A280 (DropSense96, Trinean). Peak fractions were pooled, and buffer was exchanged into DPBS. Centrifugal filters (EMD Millipore, cat. No. UFC803024) were equilibrated in DPBS at 4,000×g for 2 mins. Purified sample was loaded, DPBS was added and the sample was spun at 4,000×g for 5-10 minute spins until total DPBS volume reached≥6 DV. The final pool was analyzed by A280.

Standard methods in molecular biology are described. See, e.g., Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N. Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Stable cell lines expressing target TSA/TAA as disclosed herein were generated by transfecting a selected host cell (i.e., CHO-K1, HEK293T) with pcDNA3.1-based plasmids expressing TSA/TAA proteins as disclosed herein using electroporation-based transfection. Geneticin was used to select the integrated cells. After 7-10 days of geneticin selection, stable clones were isolated by FACS. After expansion, the stable clones were further confirmed for expression of the TSA/TAA targets by flow cytometry.

An in-house control anti-CD137 antibody based on the anti-CD137 antibody (Urelumab) referred to herein as “Urelumab-NR” was prepared based on the publicly available information published in U.S. Pat. No. 7,288,638 (VH SEQ ID NO: 3 and VL SEQ ID NO: 6 therein).

The sequences for the heavy and light chain variable regions for hybridoma clones were determined as described below. Total RNA was extracted from 1-2×10⁶ hybridoma cells using the RNeasy Plus Mini Kit from Qiagen (Germantown, MD, USA). CDNA was generated by performing 5′ RACE reactions using the SMARTer RACE 5′/3′ Kit from Takara (Mountainview, CA, USA). PCR was performed using the Q5 High-Fidelity DNA Polymerase from NEB (Ipswich, MA, USA) to amplify the variable regions from the heavy and light chains using the Takara Universal Primer Mix in combination with gene specific primers for the 3′ mouse constant region of the appropriate immunoglobulin. The amplified variable regions for the heavy and light chains were run on 2% agarose gels, the appropriate bands excised and then gel purified using the Mini Elute Gel Extraction Kit from Qiagen. The purified PCR products were cloned using the Zero Blunt PCR Cloning Kit from Invitrogen (Carlsbad, CA, USA), transformed into Stellar Competent E. Coli cells from Takara and plated onto LB Agar+50 μg/ml kanamycin plates. Direct colony Sanger sequencing was performed by GeneWiz (South Plainfield, NJ, USA). The resulting nucleotide sequences were analyzed using IMGT V-QUEST to identify productive rearrangements and analyze translated protein sequences. CDR determination was based on Kabat numbering.

Recombinant monoclonal or bispecific binding proteins were expressed and purified as follows: respective heavy or light chains were PCR amplified or synthesized and cloned into a pcDNA3.4-based expression vector, which harbors the constant region derived from human IgG1 (Uniprot P01857) or human Kappa light chain (UniProt P01834) or human Lambda light chain (UniProt P0DOY2). Paired heavy chain- and light chain-expressing plasmids were transfected into Expi293 cells (Thermo Fisher Scientific) following provider's Expi293 expression system protocol. Five days after transfection culture supernatants were collected by centrifugation. Antibodies were purified by 1-step affinity purification using Protein A column and buffer exchanged to 20 mM Sodium Acetate, pH 5.0 or PBS pH 7.2.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard techniques for characterizing ligand/receptor interactions are available. See, e.g., Coligan et al. (2001) Current Protocols in Immunology, Vol. 4, John Wiley, Inc., New York. Standard methods of antibody functional characterization appropriate for the characterization of antibodies with particular mechanisms of action are also well known to those of skill in the art.

Software packages and databases for determining, e.g., antigenic fragments, leader sequences, protein folding, functional domains, CDR annotation, glycosylation sites, and sequence alignments, are available.

Reference sequences utilized herein are illustrated in Table 9

TABLE 9
Reference Sequences

| SEQ | | |
|---|---|---|
| ID | | |
| NO: | NCBI Ref. Seq | Amino Acid Sequence |
| SEQ | NP_001002026.1 | MAVTACQGLGFVVSLIGIAGIIAAT |
| ID | Human | CMDQWSTQDLYNNPVTAVFNYQGLW |
| NO: | Claudin-18 | RSCVRESSGFTECRGYFTLLGLPAM |
| 72 | isoform 2 | LQAVRALMIVGIVLGAIGLLVSIFA |
| | | LKCIRIGSMEDSAKANMTLTSGIMF |
| | | IVSGLCAIAGVSVFANMLVTNFWMS |
| | | TANMYTGMGGMVQTVQTRYTFGAAL |
| | | FVGWVAGGLTLIGGVMMCIACRGLA |
| | | PEETNYKAVSYHASGHSVAYKPGGF |
| | | KASTGFGSNTKNKKIYDGGARTEDE |
| | | VQSYPSKHDYV |
| SEQ | XP_001114708.1 | MAVTACQGLGFVVSLIGIAGIIAAT |
| ID | Rhesus | CMDQWSTQDLYNNPVTAVFNYQGLW |
| NO: | Claudin-18 | RSCVRESSGFTECRGYFTLLGLPAM |
| 73 | isoform X2 | LQAVRALMIVGIVLGAIGLLVSIFA |
| | | LKCIRIGSMEDSAKANMTLTSGIMF |
| | | IVSGLCAIAGVSVFANMLVTNFWMS |
| | | TANMYTGMGGMVQTVQTRYTFGAAL |
| | | FVGWVAGGLTLIGGVMMCIACRGLA |
| | | PEETNYKAVSYHASGHSVAYKPGGF |
| | | KASTGFGSNTKNKKTYDGGAHTEDE |
| | | LQSYPSKHDYV |
| SEQ | XP_032766108.1 | MSVTACQGLGFVVSLIGFAGIIAAT |
| ID | Rat Claudin- | CMDQWSTQDLYNNPVTAVFNYQGLW |
| NO: | 18 isoform X2 | RSCVRESSGFTECRGYFTLLGLPAM |
| 74 | | LQAVRALMIVGIVLGVIGILVSIFA |
| | | LKCIRIGSMDDSAKAKMTLTSGIMF |
| | | IISGVCAIIGVSVFANMLVTNFWMS |
| | | TANMYSGMGGMVQTVQTRYTFGAAL |
| | | FVGWIAGGLTLIGGVMMCIACRGLT |
| | | PDDRNFKAVSYHASGQNVAYKPGGF |
| | | KASTGFGSNARNKKIYDGGARTEDD |
| | | EQSHPTKYDYV |
| SEQ | NP_067018.2 | MASAGMQILGVVLTLLGWVNGLVSC |
| ID | Human Claudin- | ALPMWKVTAFIGNSIVVAQVVWEGL |
| NO: | 6 precursor | WMSCVVQSTGQMQCKVYDSLLALPQ |
| 75 | | DLQAARALCVIALLVALFGLLVYLA |
| | | GAKCTTCVEEKDSKARLVLTSGIVF |
| | | VISGVLTLIPVCWTAHAIIRDFYNP |
| | | LVAEAQKRELGASLYLGWAASGLLL |
| | | LGGGLLCCTCPSGGSQGPSHYMARY |
| | | STSAPAISRGPSEYPTKNYV |
| SEQ | XP_005591080.1 | MASAGMQILGVVLTLLGWVNGLVSC |
| ID | Cyno Claudin- | ALPMWKVTAFIGNSIVVAQVVWEGL |
| NO: | 6 +predicted+ | WMSCVVQSTGQMQCKVYDSLLALPQ |
| 76 | | DLQAARALCVIALLVALFGLLVYLA |
| | | GAKCTTCVEEKDSKARLVLTSGIVF |
| | | VISGVLTLIPVCWTAHAIIRDFYNP |
| | | LVAEAQKRELGASLYLGWAASGLLL |
| | | LGGGLLCCTCPSGGSRGPSHYMARY |
| | | STSAPAISRGPSEYPTKNYV |
| SEQ | NP_061247.1 | MASTGLQILGIVLTLLGWVNALVSC |
| ID | Murine claudin- | ALPMWKVTAFIGNSIVVAQMVWEGL |
| NO: | 6 precursor | WMSCVVQSTGQMQCKVYDSLLALPQ |
| 77 | | DLQAARALCVVTLLIVLLGLLVYLA |
| | | GAKCTTCVEDRNSKSRLVLISGIIF |
| | | VISGVLTLIPVCWTAHSIIQDFYNP |
| | | LVADAQKRELGASLYLGWAASGLLL |
| | | LGGGLLCCACSSGGTQGPRHYMACY |
| | | STSVPHSRGPSEYPTKNYV |
| SEQ | NP_112178.2 | MPLSLGAEMWGPEAWLLLLLLLASF |
| ID | Human Nectin-4 | TGRCPAGELETSDVVTVVLGQDAKL |
| NO: | precursor | PCFYRGDSGEQVGQVAWARVDAGEG |
| 78 | | AQELALLHSKYGLHVSPAYEGRVEQ |
| | | PPPPRNPLDGSVLLRNAVQADEGEY |
| | | ECRVSTFPAGSFQARLRLRVLVPPL |
| | | PSLNPGPALEEGQGLTLAASCTAEG |
| | | SPAPSVTWDTEVKGTTSSRSFKHSR |
| | | SAAVTSEFHLVPSRSMNGQPLTCVV |
| | | SHPGLLQDQRITHILHVSFLAEASV |
| | | RGLEDQNLWHIGREGAMLKCLSEGQ |
| | | PPPSYNWTRLDGPLPSGVRVDGDTL |
| | | GFPPLTTEHSGIYVCHVSNEFSSRD |
| | | SQVTVDVLDPQEDSGKQVDLVSASV |
| | | VVVGVIAALLFCLLVVVVVLMSRYH |
| | | RRKAQQMTQKYEEELTLTRENSIRR |
| | | LHSHHTDPRSQPEESVGLRAEGHPD |
| | | SLKDNSSCSVMSEEPEGRSYSTLTT |
| | | VREIETQTELLSPGSGRAEEEEDQD |
| | | EGIKQAMNHFVQENGTLRAKPTGNG |
| | | IYINGRGHLV |
| SEQ | XP_005541277.1 | MPLSLGAEMWGPEAWLLLLLLLASF |
| ID | Cyno Nectin-4 | TGRCPAGELETSDVVTVVLGQDAKL |
| NO: | [predicted] | PCFYRGDSGEQVGQVAWARADAGEG |
| 79 | | AQELALLHSKYGLHVSPAYEGRVEQ |
| | | PPPPRNPLDGSVLLRNAVQADEGEY |
| | | ECRVSTFPAGSFQARLRLRVLVPPL |
| | | PSLNPGPALEEGQGLTLAASCTAEG |
| | | SPAPSVTWDTEVKGTTSSRSFKHSR |
| | | SAAVTSEFHLVPSRSMNGQPLTCVV |
| | | SHPGLLQDQRITHILHVSFLAEASV |
| | | RGLEDQNLWHVGREGAMLKCLSEGQ |
| | | PPPSYNWTRLDGPLPSGVRVDGDTL |
| | | GFPPLTTEHSGIYVCHVSNEFSSRD |
| | | SQVTVDVLDPQEDSGKQVDLVSASV |
| | | VVVGVIAALLFCLLVVVVVLMSRYH |
| | | RRKAQQMTQKYEEELTLTRENSIRR |
| | | LHSHHTDPRSQPEESVGLRAEGHPD |
| | | SLKDNSSCSVMSEEPEGRSYSTLTT |
| | | VREIETQTELLSPGSGRTEEEEDQD |
| | | EGIKQAMNHFVQENGTLRAKPTGNG |
| | | IYINGRGHLV |
| SEQ | NP_001102546. | MPLSLGAEMWGPEAWLLLLFLASFT |
| ID | 1 Rat Nectin-4 | GRYSAGELETSDLVTVVLGQDAKLP |
| NO: | precursor | CFYRGDPDEQVGQVAWARVDPNEGT |
| 80 | | RELALLHSKYGLHVSPAYEDRVEQP |
| | | PPPRDPLDGSILLRNAVQADEGEYE |
| | | CRVSTFPAGSFQARMRLRVLVPPLP |
| | | SLNPGPPLEEGQGLTLAASCTAEGS |
| | | PAPSVTWDTEVKGTQSSRSFKHSRS |
| | | AAVTSEFHLVPSRSMNGQPLTCVVS |
| | | HPGLLQDQRITHTLQVAFLAEASVR |
| | | GLEDQNLWHVGREGATLKCLSEGQP |
| | | PPKYNWTRLDGPLPSGVRVKGDTLG |
| | | FPPLTTEHSGVYVCHVSNELSSRAS |
| | | QVTVEVLDPEDPGKQVDLVSASVVV |
| | | VGVIAALLFCLLVVVVVLMSRYHRR |
| | | KAQQMTQKYEEELTLTRENSIRRLH |
| | | SHHTDPRSQPEESVGLRAEGHPDSL |
| | | KDNSSCSVMSEEPEGRSYSTLTTVR |
| | | EIETQTELLSPGSGRTEEEDDQDEG |
| | | IKQAMNHFVQENGTLRAKPTGNGIY |
| | | INGRGHLV |
| SEQ | NP_082169.2 | MPLSLGAEMWGPEAWLRLLFLASFT |
| ID | Murine Nectin-4 | GQYSAGELETSDVVTVVLGQDAKLP |
| NO: | isoform a | CFYRGDPDEQVGQVAWARVDPNEGI |
| 81 | | RELALLHSKYGLHVNPAYEDRVEQP |
| | | PPPRDPLDGSVLLRNAVQADEGEYE |
| | | CRVSTFPAGSFQARMRLRVLVPPLP |
| | | SLNPGPPLEEGQGLTLAASCTAEGS |
| | | PAPSVTWDTEVKGTQSSRSFTHPRS |
| | | AAVTSEFHLVPSRSMNGQPLTCVVS |
| | | HPGLLQDRRITHTLQVAFLAEASVR |
| | | GLEDQNLWQVGREGATLKCLSEGQP |
| | | PPKYNWTRLDGPLPSGVRVKGDTLG |
| | | FPPLTTEHSGVYVCHVSNELSSRDS |
| | | QVTVEVLDPEDPGKQVDLVSASVII |
| | | VGVIAALLFCLLVVVVVLMSRYHRR |
| | | KAQQMTQKYEEELTLTRENSIRRLH |
| | | SHHSDPRSQPEESVGLRAEGHPDSL |
| | | KDNSSCSVMSEEPEGRSYSTLTTVR |
| | | EIETQTELLSPGSGRTEEDDDQDEG |
| | | IKQAMNHFVQENGTLRAKPTGNGIY |
| | | INGRGHLV |

### Example 1: Generation of Binding Proteins that Bind CD137 and Claudin 6, Claudin 18.2, or Nectin-4

Fully human anti-human CD137, Claudin 6, and Claudin 18.2, antibodies were generated by immunizing human Ig transgenic mice, Trianni mice that express human antibody VH and VL genes (see, e.g., WO 2013/063391, TRIANNI® mice).

Immunization-TRIANNI mice described above were immunized by injection either with recombinant human proteins, stable cell lines expressing target proteins, or DNA via intraperitoneally (IP), subcutaneously (SC), the base of tail or footpad injections.

Mouse anti-Nectin-4 antibodies were generated by immunizing Balb/c mice with recombinant human Nectin-4 protein either intraperitoneally (IP) subcutaneously (SC), or base of tail or footpad injections.

The immune response was monitored by retroorbital bleeds. The plasma was screened by ELISA, flow cytometry (FACS) or Imaging (as described below). Mice with sufficient anti-CD137, Claudin 6, Claudin 18.2, or Nectin-4 titers were used for fusions. Mice were boosted intraperitoneally, at the base of the tail, footpad or intravenously with the immunogen before sacrifice and removal of the spleen and lymph nodes.

To select mice producing antibodies that bound CD137, Claudin 6, Claudin 18.2, or Nectin-4, sera from immunized mice were screened by ELISA, FACS or imaging for binding to human CD137, Claudin 6, Claudin 18.2, or Nectin-4 protein, respectively.

For ELISA, briefly, an ELISA plate coated with recombinant human CD137, or Nectin-4 was incubated with dilutions of serum from immunized mice for one hour at room temperature, the assay plate was washed, and specific antibody binding was detected with HRP-labeled anti-mouse IgG antibody (Jackson ImmunoResearch, catalog number: 115-036-071) after one hour incubation at room temperature, washed, and followed by ABTS substrate (Moss, catalog number: ABTS-1000) incubation for 30 minutes at room temperature. The plate was read using an ELISA plate reader (Biotek).

For FACS, briefly, CD137, Claudin 6, Claudin 18.2, or Nectin-4-expressing HEK293T or CHO-K1 cells or parental HEK293T or CHO-K1 cells were incubated with dilutions of serum from immunized mice for 2 hours at 4° C. Cells were fixed with 2% PFA (Alfa Aesar, catalog number: J61899) for 15 minutes at 4° C. and then washed. Specific antibody binding was detected with Alexa 647 labeled goat anti-mouse IgG antibody (ThermoFisher Scientific, catalog number: A21235) after one-hour incubation at 4° C. Flow cytometric analyses were performed on a flow cytometry instrument (Intellicyte, IQue plus, Sartorius).

In addition, mice serum was tested by imaging. Briefly, CD137, Claudin 6, Claudin 18.2, or Nectin-4-expressing HEK293T or CHO-K1 cells were incubated with dilutions of serum from immunized mice. Cells were washed, fixed with paraformaldehyde, washed, specific antibody binding was detected with secondary Alexa488 goat anti-mouse antibody and Hoechst (Invitrogen). Plates were scanned and analyzed on an imaging machine (Cytation 5, Biotek).

To generate hybridomas producing human antibodies to CD137, Claudin 6, Claudin 18.2, or Nectin-4, splenocytes and lymph node cells were isolated from an immunized mouse and fused to an appropriate immortalized cell line, such as a mouse myeloma cell line. The resulting hybridomas were screened for the production of antigen-specific antibodies. For example, single cell suspensions of splenocytes, lymph node cells from immunized mice were fused to an equal number of Sp2/0 non-secreting mouse IgG myeloma cells (ATCC, CRL 1581) by electrofusion. Cells were plated in flat bottom 96-well tissue culture plates, followed by about one week of incubation in selection medium (HAT medium), then switched to hybridoma culture media. Approximately 10-14 days after cell plating, supernatants from individual wells were screened by ELISA, Imaging or FACS as described above. The antibody secreting hybridomas were transferred to 24-well plates, screened again, and if still positive for anti-CD137, Claudin 6, Claudin 18.2, or Nectin-4, the positive hybridomas were subcloned by sorting using a single cell sorter. The subclones were screened again by ELISA, Imaging or FACS as described above. The stable subclones were then cultured in vitro to generate small amounts of antibodies for purification and characterization.

A murine anti-Nectin-4 antibody was humanized by CDR grafting. Briefly, the VH and VL of the 1925Ab4 was used as query respectively to search against human antibody germline sequences for the most similar human framework regions. Murine CDRs (based on Kabat numbering) were grafted into the identified human antibody frameworks. Several pairs of the humanized VH and VL variants were expressed and purified, and one pair (1925Ab4 VH (Hz) and 1925Ab4 VL (Hz) with the highest binding affinity to human Nectin-4 was used to construct bispecific antibodies.

### Example 2: Molecular Design and Production of a TAA/CD137 Bispecific (BsAb_A)

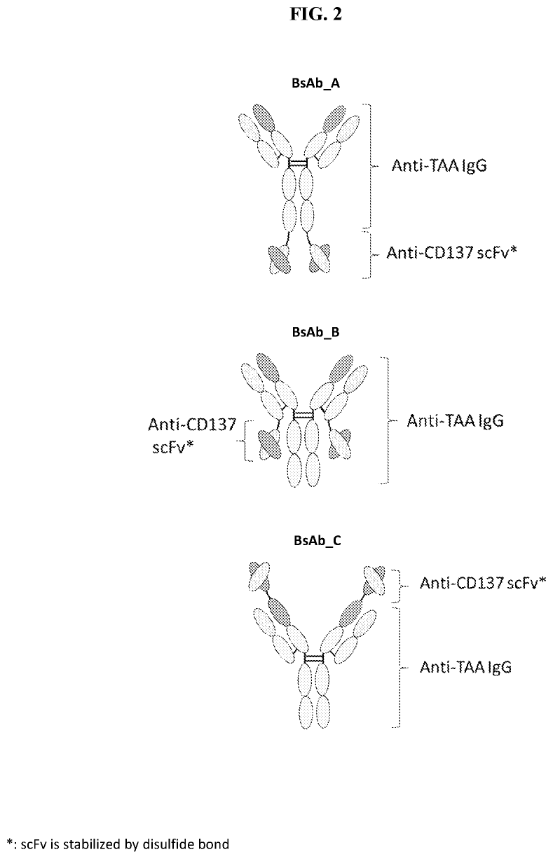

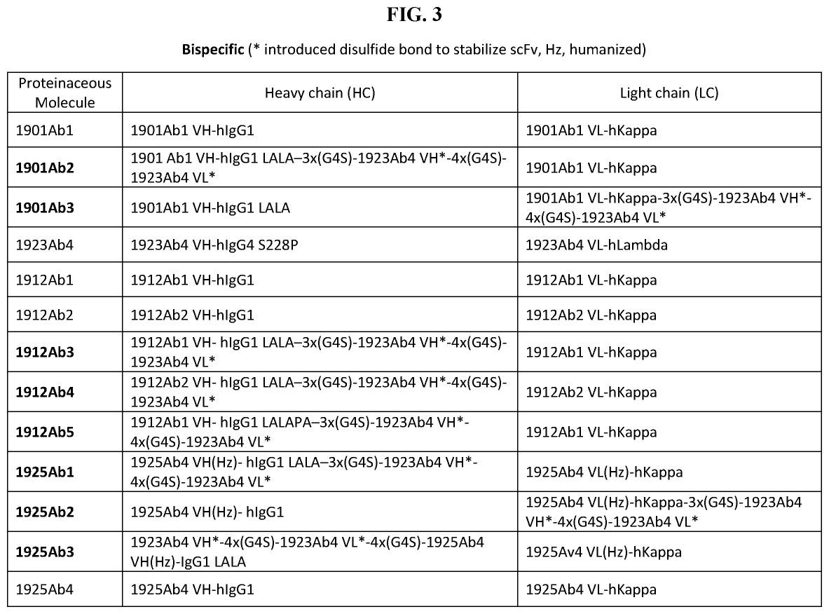

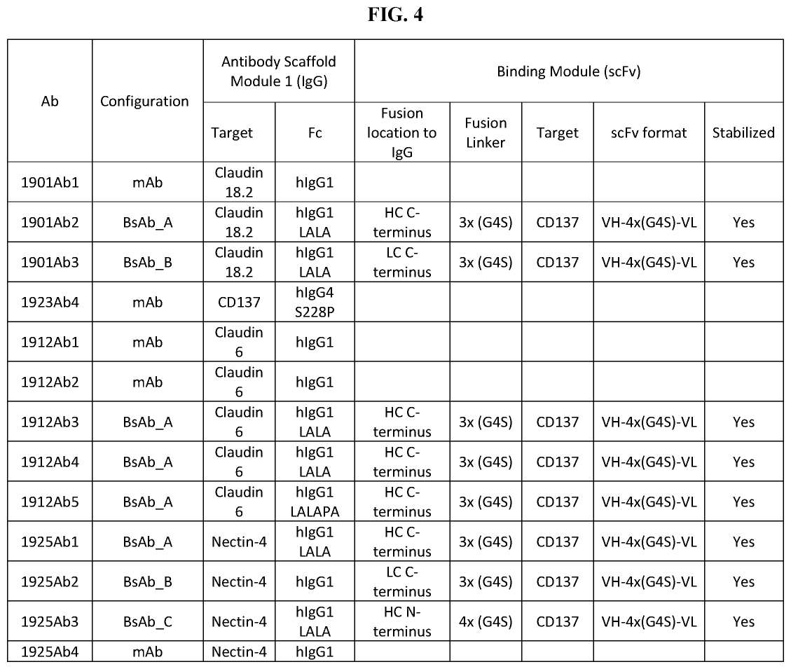

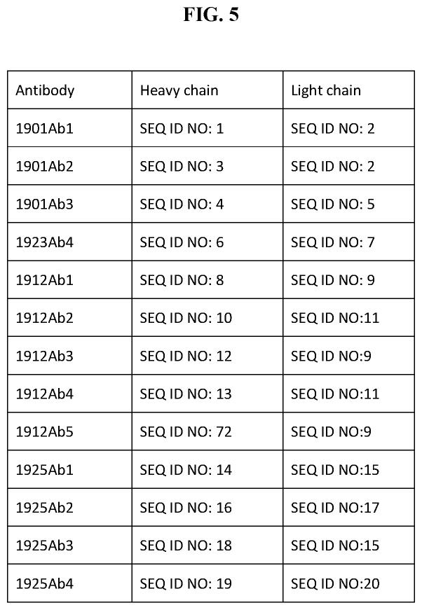

As a representative example of a binding protein that binds TAA, a symmetrical bispecific (Claudin 6× CD137) characterized by the molecular format depicted in FIG. 2 BsAb_A comprising the subunit/components summarized in FIGS. 3 and 4 was prepared:

### 1912Ab3

1. Heavy Chain: SEQ ID NO: 12 comprising the components: heavy chain of anti-Claudin 6 antibody, linker and anti-CD137 scFv (VH-VL with CC) (N→C); and
2. Light Chain: SEQ ID NO: 9 comprising an anti-Claudin 6 antibody light chain

A DNA segment 1 having a polynucleotide sequence encoding the heavy chain component of 1912Ab3 (SEQ ID NO: 12) was inserted into an expression vector, and a DNA segment 2 having a polynucleotide sequence encoding the light chain of 1912Ab3 (SEQ ID NO: 9) was inserted in the expression vector.

### 1912Ab5

1. Heavy Chain: SEQ ID NO: 72 comprising the components: heavy chain of anti-Claudin 6 antibody, linker and anti-CD137 scFv (VH-VL with CC) (N→C); and
2. Light Chain: SEQ ID NO: 9 comprising an anti-Claudin 6 antibody light chain.

In an alternative example, a DNA segment 1 having a polynucleotide sequence encoding the heavy chain component of 1912Ab5 (SEQ ID NO: 72) was inserted into an expression vector, and a DNA segment 2 having a polynucleotide sequence encoding the light chain of 1912Ab5 (SEQ ID NO:9) was inserted in the expression vector.

The constructed expression vectors were transiently expressed in Expi293 cells (ThermoFisher), cultured in Expi293 Expression medium under the condition of 37° C. for 5 days in a CO2 incubator. The bispecific antibody was purified from the cell culture supernatant by recombinant protein A affinity chromatography (Hitrap Mabselect SuRe, GE) and second step purification by Ion exchange chromatography or gel filtration chromatography if necessary. SDS-PAGE (BiRad), size exclusion HPLC (Agilent, 1100 series) analysis with SE-HPLC column (TOSO, G3000SWXL) and CE-SDS (SCIEX, PA800 Plus) were performed to detect and confirm the size and purity of bispecific antibody. Purified proteins were buffer-exchanged into the desired buffer and concentrated by ultrafiltration using an Amicon Ultra 15 30K device, and protein concentrations were estimated using dropsense (Unchained Lab). The transient transfection could be used in a two-vector system or with a one-vector system that contains both heavy and light chain components in one single vector. Alternatively, the bispecific antibody could be purified from the supernatant of stable CHO expression cell lines.

### Example 3: Molecular Design and Production of a TAA/CD137 Bispecific (BsAb_B)

As a representative example of a binding protein that binds TAA, a symmetrical bispecific (Claudin 18.2× CD137) characterized by the molecular format depicted in FIG. 2 BsAb_B comprising the subunit/components summarized in FIGS. 3 and 4 was prepared:

### 1901Ab3

1. Heavy Chain: SEQ ID NO: 4 comprising the components: heavy chain of anti-Claudin 18.2 antibody; and
2. Light Chain: SEQ ID NO: 5 comprising an anti-Claudin 18.2 antibody light chain, linker and anti-CD137 scFv (VH-VL with CC) (N→C)

A DNA segment 1 having a polynucleotide sequence encoding the heavy chain component of 1901Ab3 (SEQ ID NO: 4) was inserted into an expression vector, and a DNA segment 2 having a polynucleotide sequence encoding the light chain of 1901Ab3 (SEQ ID NO: 5) was inserted in the expression vector.

The constructed expression vectors were transiently expressed in Expi293 cells (ThermoFisher), cultured in Expi293 Expression medium under the condition of 37° C. for 5 days in a CO2 incubator. The bispecific antibody was purified from the cell culture supernatant by recombinant protein A affinity chromatography (Hitrap Mabselect SuRe, GE) and second step purification by Ion exchange chromatography or gel filtration chromatography if necessary. SDS-PAGE (BiRad), size exclusion HPLC (Agilent, 1100 series) analysis with SE-HPLC column (TOSO, G3000SWXL) and CE-SDS (SCIEX, PA800 Plus) were performed to detect and confirm the size and purity of bispecific antibody. Purified proteins were buffer-exchanged into the desired buffer and concentrated by ultrafiltration using an Amicon Ultra 15 30K device, and protein concentrations were estimated using dropsense (Unchained Lab). The transient transfection could be used in a two-vector system or with a one-vector system that contains both heavy and light chain components in one single vector. Alternatively, the bispecific antibody could be purified from the supernatant of stable CHO expression cell lines.

### Example 4: Molecular Design and Production of a TAA/CD137 Bispecific (BsAb_C)

As a representative example of a binding protein that binds TAA, a symmetrical bispecific (Nectin-4× CD137) characterized by the molecular format depicted in FIG. 2 BsAb_C comprising the subunit/components summarized in FIGS. 3 and 4 was prepared:

### 1925Ab3

1. Heavy Chain: SEQ ID NO: 18 comprising the components: anti-CD137 scFv (VH-VL with CC) (N→C), linker, and heavy chain of a humanized anti-Nectin-4 antibody; and
2. Light Chain: SEQ ID NO: 15 comprising a humanized Nectin-4 antibody light chain

A DNA segment 1 having a polynucleotide sequence encoding the heavy chain component of 1925Ab3 (SEQ ID NO: 18) was inserted into an expression vector, and a DNA segment 2 having a polynucleotide sequence encoding the light chain of 1925Ab3 (SEQ ID NO: 15) was inserted in the expression vector.

The constructed expression vectors were transiently expressed in Expi293 cells (ThermoFisher), cultured in Expi293 Expression medium under the condition of 37° C. for 5 days in a CO2 incubator. The bispecific antibody was purified from the cell culture supernatant by recombinant protein A affinity chromatography (Hitrap Mabselect SuRe, GE) and second step purification by Ion exchange chromatography or gel filtration chromatography if necessary. SDS-PAGE (BiRad), size exclusion HPLC (Agilent, 1100 series) analysis with SE-HPLC column (TOSO, G3000SWXL) and CE-SDS (SCIEX, PA800 Plus) were performed to detect and confirm the size and purity of bispecific antibody. Purified proteins were buffer-exchanged into the desired buffer and concentrated by ultrafiltration using an Amicon Ultra 15 30K device, and protein concentrations were estimated using dropsense (Unchained Lab). The transient transfection could be used in a two-vector system or with a one-vector system that contains both heavy and light chain components in one single vector. Alternatively, the bispecific antibody could be purified from the supernatant of stable CHO expression cell lines.

### Example 5: Binding of CLDN6/CD137 BsAbs to Claudin 6 on the Cell Surface

Bispecific CLDN6/CD137 binding proteins were generated, produced, and purified as described in Example 4. To examine the binding activity of BsAbs 1912Ab3 and 1912Ab4 to Claudin 6, an immunofluorescence binding assay was performed using NEC8 WT cells expressing endogenous human Claudin 6 on the cell surface or NEC8 Claudin 6 KO cells. The NEC8 Claudin 6 KO cells were generated by CRISPR gene editing technology. These cells were cultured in RPMI with 10% FBS. On the day of the experiment, the cells were collected, washed, and stained with the BsAbs 1912Ab3 and 1912Ab4, and mAbs 1912Ab1 and 1912Ab2 at 4° C. for 2 hours followed by fixing cells for 15 minutes at room temperature. The fixed cells were washed with PBS three times following by staining at room temperature for 1 hour with Alexa Fluor® 488 Goat Anti-Human IgG antibody (Invitrogen, Cat #: A-11013) for detection. The binding signal was assessed by quantifying the fluorescence intensity using iQue Screener PLUS (Sartorius, MI).

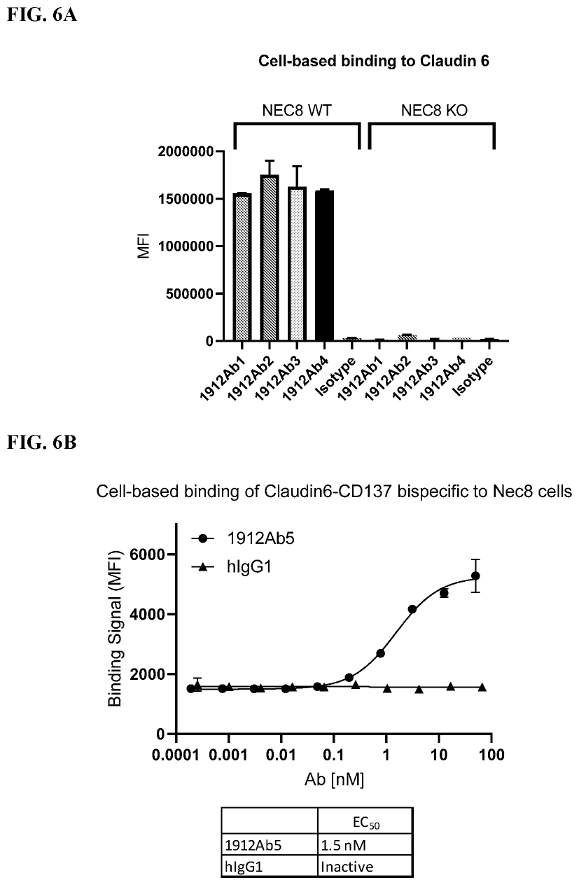

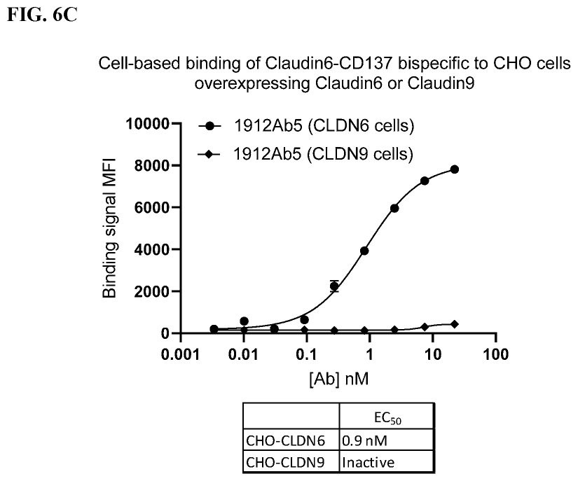

As shown in FIG. 6A, at the concentration of 10 μg/ml, the disclosed bispecific binding proteins including 1912Ab3 and 1912Ab4 bound similarly to human Claudin 6 on the cell surface of NEC8 cells compared to the monospecific control antibodies, 1912Ab1 and 1912Ab2. No binding was detected when Claudin 6 gene was deleted by CRISPR gene editing technology. The result confirmed that the binding of bispecific binding proteins on NEC8 WT cells is specific through Claudin 6 expressing on the cell surface of NEC8 cells. The concentration-dependent binding curve of 1912Ab5 to Claudin6 is shown in FIG. 6B. 1912Ab5 binds to NEC8 cells with a binding EC₅₀ value of 1.5 nM.

Because of the high homology between Claudin6 and Claudin9 and the normal cell expressing profile of Claudin9, a highly selective Claudin6 antibody is desirable for treating cancer and minimizing safety issues. To evaluate the binding selectivity of Claudin6 over Claudin9, two CHO cell lines overexpressing either Claudin6 or Claudin9 were used in an image based cell binding assay. As shown in FIG. 6C, 1912Ab5 binds to CHO-Claudin6 cells without bindng to CHO-Claudin9 cells.

### Example 6: Binding of CLDN6/CD137 BsAbs to CD137

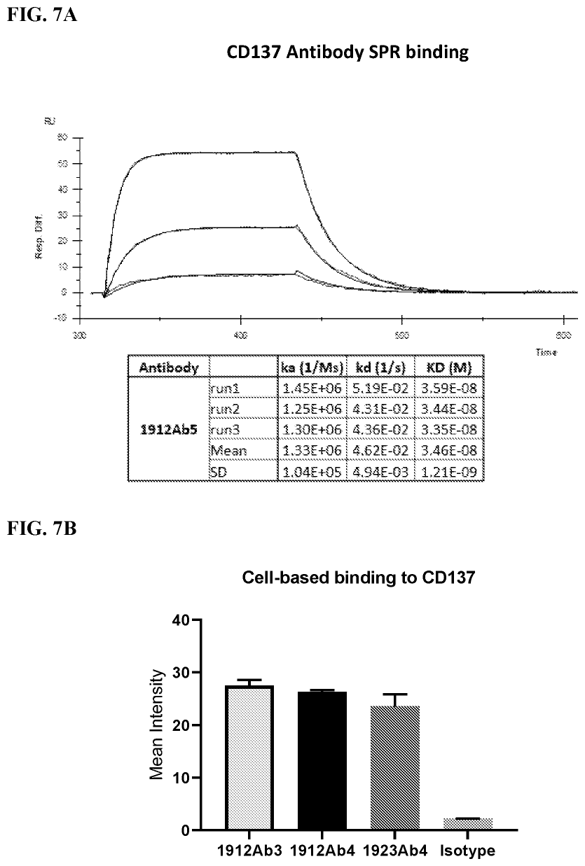

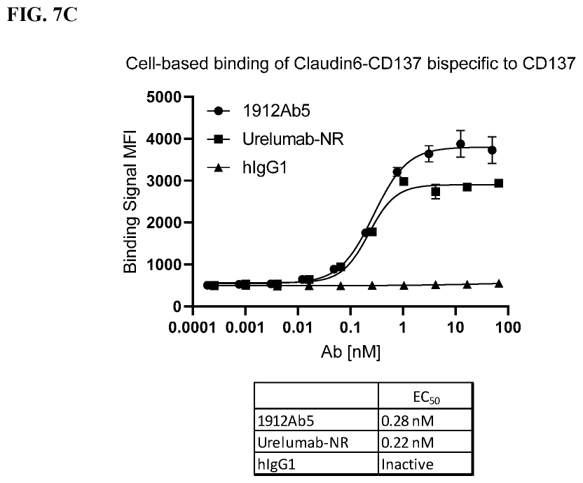

The Binding of Claudin6-CD137 BsAbs to CD137 was measured by a SPR assay and an immunofluorescence imaging assay. As shown in FIG. 7A, 1912Ab5 has a desired fast-on fast-off kinetics when binding to human CD137. From three experiments, 1912Ab5 had an average ka value of 1.33E+06 (1/Ms), and an average kd value of 4.62E-02 (1/s). The average KD was 3.46E+08M. The intermittent binding could potentially lower the risk of over-stimulating T cells and causing T cell exhaustion.

HEK293T cells stably transfected with human CD137 expression construct were used in the cell-based binding assay to evaluate the CD137 binding affinity. The cells were plated in complete media containing DMEM with 10% FBS, then incubated overnight at 37° C. Cells were stained with the testing antibodies at 4° C. for 2 hours followed by fixing cells for 15 minutes at room temperature. The fixed cells were washed with PBS three times following by staining at room temperature for 1 hour with Alexa Fluor® 488 Goat Anti-Human IgG (H+L) secondary antibody (Invitrogen, Cat #: A-11013) for detection. The binding signal was assessed by imaging the cells and quantifying the fluorescence intensity using Cytation Imager (Biotek, VT).

The result shown in FIG. 7B indicates that bispecific antibodies 1912Ab3 and 1912Ab4, and the monospecific control antibody 1923Ab4, at the concentration of 10 μg/ml, bound to human CD137 at a similar level. The concentration-dependent binding curve of 1912Ab5 is shown in FIG. 7C. 1912Ab5 binds to HEK293-Claudin6 cells with a binding EC₅₀ value of 0.28 nM, the benchmark control Urelumab-NR binding EC₅₀ was 0.22 nM.

### Example 7: Claudin 6 Dependent Activation of CD137 Signaling

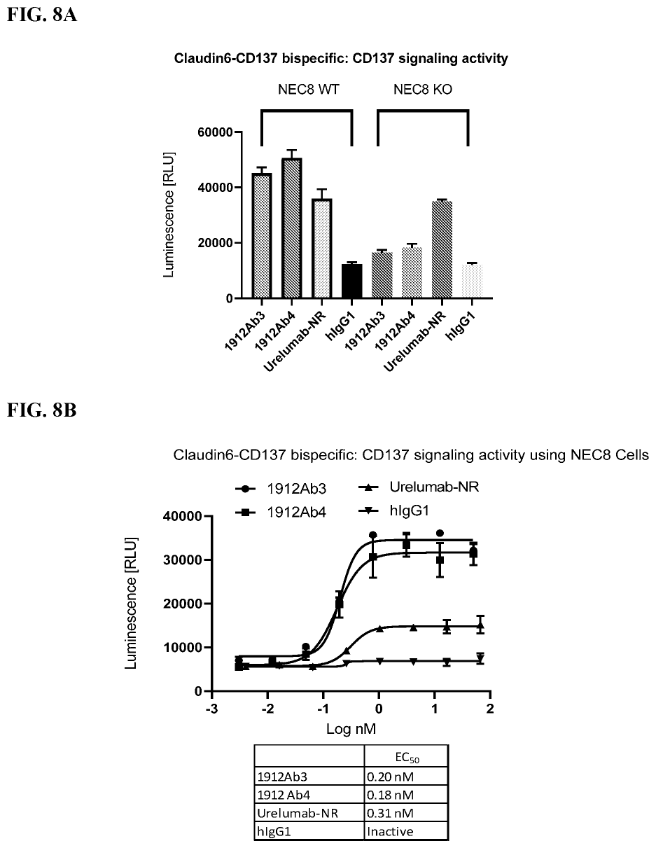

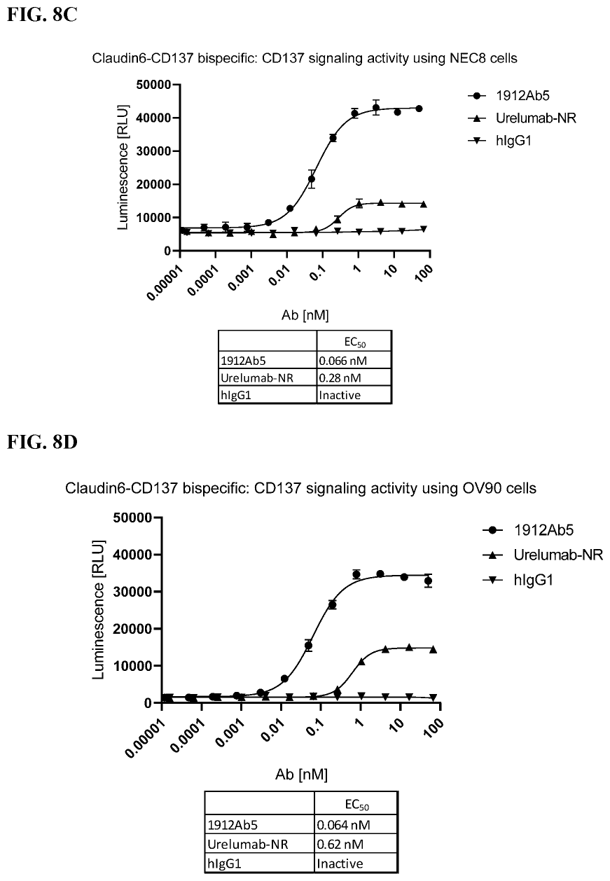

The CLDN6/CD137 BsAbs were evaluated for their ability to induce Claudin 6 dependent CD137 agonism. In brief, Jurkat T reporter cell line stably expressing CD137 and containing NFKB-Luc report was used to quantify CD137 signaling and NEC8 WT cells, which expressed endogenous Claudin 6 on the cell surface, was used as target cell to provide Claudin 6. NEC8 Claudin 6 KO cells were used as a negative control to show the Claudin 6 dependency. The disclosed antibodies 1912Ab3 and 1912Ab4 are bispecific antibodies that bind to both Claudin 6 and CD137. Monospecific antibody Urelumab-NR only binds to CD137 and is used as a control antibody. The Jurkat T reporter cells were co-cultured with either NEC8 WT or Claudin 6 KO cells and were stimulated with the disclosed binding proteins for 16 hours at 37° C. with 5% CO₂. ONE-Glo™ luciferase reagent (Promega, Cat #: E6130) was added and the plate was incubated at room temperature for 10 minutes. The luminescence signal was measured by a Synergy Neo2 plate reader (Biotek) and data was analyzed by GraphPad Prism. FIG. 8A demonstrates that only Urelumab-NR activated CD137 signaling in both NEC8 WT and Claudin 6 KO target cells. 1912Ab3 and 1912Ab4 induced stronger CD137 signaling than Urelumab-NR in the presence of NEC8 WT cells. Only background activity was detected when in the Claudin 6 knock-out NEC8 cells.

The dose response curves of 1912Ab3, 1912Ab4 and Urelumab-NR to induce CD137 signaling in the presence of NEC8 WT cells were shown in FIG. 8B. The EC₅₀ values (potency) of 1912Ab3, 1912Ab4 and Urelumab-NR were 0.20 nM, 0.18 nM and 0.31 nM, respectively. Both 1912Ab3 and 1912Ab4 demonstrated better efficacy (higher Emax) than Urelumab-NR.

Similarly, 1912Ab5 and Urelumab-NR were evaluated in the CD137 signaling assay using either NEC8 cells (FIG. 8C) or OV90 cells (FIG. 8D). As shown in the figures, 1912Ab5 induced a dose-dependent CD137 signaling with EC₅₀ values (potency) of 0.066 nM and 0.064 nM, respectively. The control antibody Urelumab-NR showed EC₅₀ values of 0.28 nM and 0.62 nM, respectively. 1912Ab5 demonstrated stronger agonism in the T cell CD137 signaling than Urelumab-NR.

### Example 8: Claudin 6 Dependent Activation of CD8 T Cells

A co-culture experiment was used to measure T cell activation by Claudin6-CD137 BsAbs. CD8 T cells from a healthy donor and NEC8 cells were used as effector and target cells, respectively. These two cells were co-cultured in RPMI1640 media supplemented with 10% FBS and 0.5 ug/ml of mouse anti-hCD3 clone OKT3 (Biolegend, Cat #: 317325). The disclosed binding proteins were added to stimulate T cells. The disclosed antibodies 1912Ab3 and 1912Ab4 are bispecific antibodies that bind to both Claudin 6 and CD137. Monospecific antibody Urelumab-NR only binds to CD137 and is used as a control antibody. The plate was incubated for 3 days at 37° C. with 5% CO₂. After 72 hours of incubation, supernatants were collected and used to measure the secreted IFNγ by AlphaLISA (PerkinElmer, Cat #: AL217C/F) using protocols according to the manufacturer's instruction. The amount of IFNγ represents T cell activation.

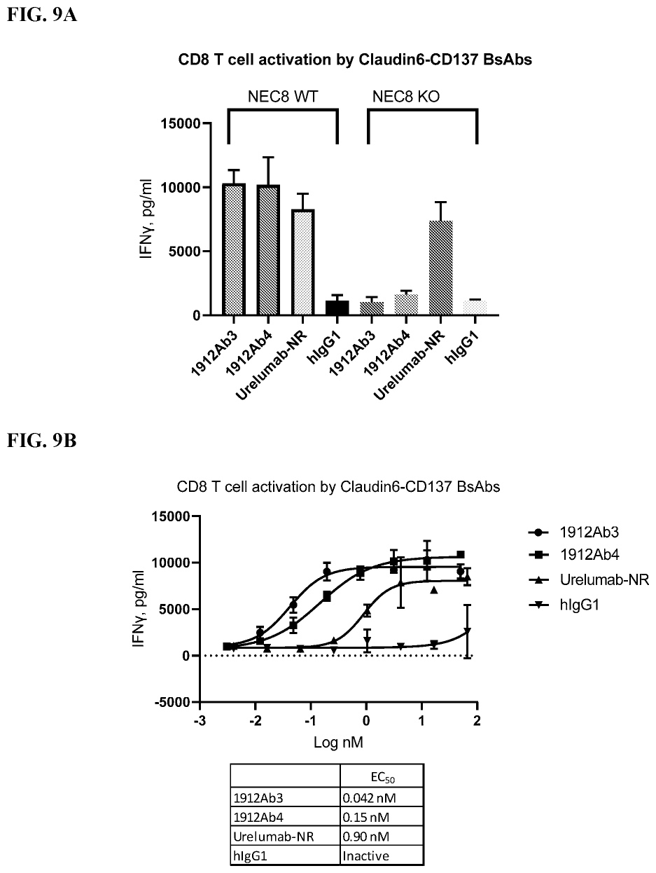

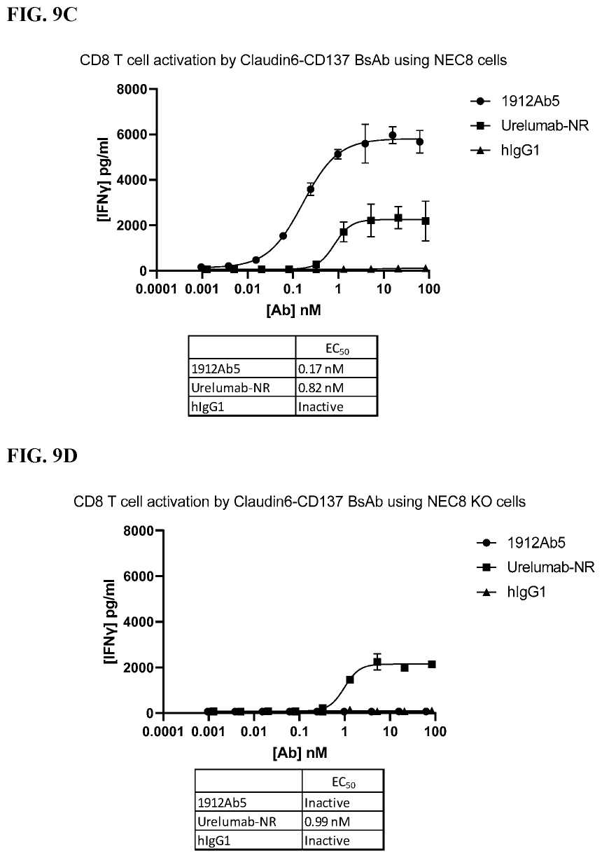

FIG. 9A shows that Urelumab-NR stimulates T cell activation independent of Claudin 6 expression. A similar level of IFNγ was detected when CD8 T cells were co-cultured with NEC8 WT or NEC8 Claudin 6 KO cells. However, 1912Ab3 and 1912Ab4 only stimulate CD8 T cell activation in the presence of NEC8 WT cells but not NEC8 Claudin 6 KO cells. This result confirms the Claudin 6 dependent T cell activating activity of the disclosed bispecific binding proteins.

The dose-response curves of 1912Ab3, 1912Ab4 and Urelumab-NR to induce CD8 T cell activation in the presence of NEC8 WT cells are shown in FIG. 9B. The EC₅₀ values (potency) of 1912Ab3, 1912Ab4 and Urelumab-NR were 0.042 nM, 0.15 nM and 0.9 nM, respectively. Both 1912Ab3 and 1912Ab4 demonstrated better potency and efficacy (higher Emax) than Urelumab-NR to induce IFNγ production, a hallmark of T cell activation.

Similarly, 1912Ab5 and Urelumab-NR were evaluated in the T cell activation assay using either NEC8 cells (FIG. 9C) or NEC8 Claudin6 KO cells (FIG. 9D). As shown in the figures, 1912Ab5 induced dose-dependent CD137 signaling with EC₅₀ values (potency) of 0.17 nM only in the presence of NEC8 wild-type cells. In contrast, the control antibody Urelumab-NR showed activity in both NEC8 wild type and NEC8 Claudin6 KO cells studies, with EC₅₀ values of 0.82 nM and 0.99 nM, respectively, indicating its activity is independent of the Claudin6 expression. 1912Ab5 demonstrated a higher level of agonism in the T cell activation study than Urelumab-NR only when the targeted tumor antigen was available.

### Example 9: Tumor Killing Activity of CD8 T Cells Induced by BsAbs CLDN6/CD137

A co-culture experiment was performed to evaluate the T cell-derived tumor killing activity (TDCC) mediated by BsAbs 1912Ab3 and 1912Ab4. In brief, CD8 T cells from a healthy donor were pre-activated with ImmunoCult™ Human CD3/CD28 T Cell Activator (Stemcell, Cat #: 10971) for 2 days. The activated cells were washed to removed CD3/CD28 activator. The activated CD8 T cells were then co-cultured with NEC8 tumor cells stably transfected with GFP expression construct and treated with the disclosed bispecific binding proteins for 108 hours. The disclosed antibodies 1912Ab3 and 1912Ab4 are bispecific antibodies which bind to both Claudin 6 and CD137. The number of cells were measured by area of green fluorescent cells, which was measured using Cytation (Biotek, VT). The percentage of killing was calculated by the following formula.

% of killing=(area of GFP cells from well without binding protein treatment-area of GFP cells from well treated with binding protein)/area of GFP cells from well without binding protein treatment*100%

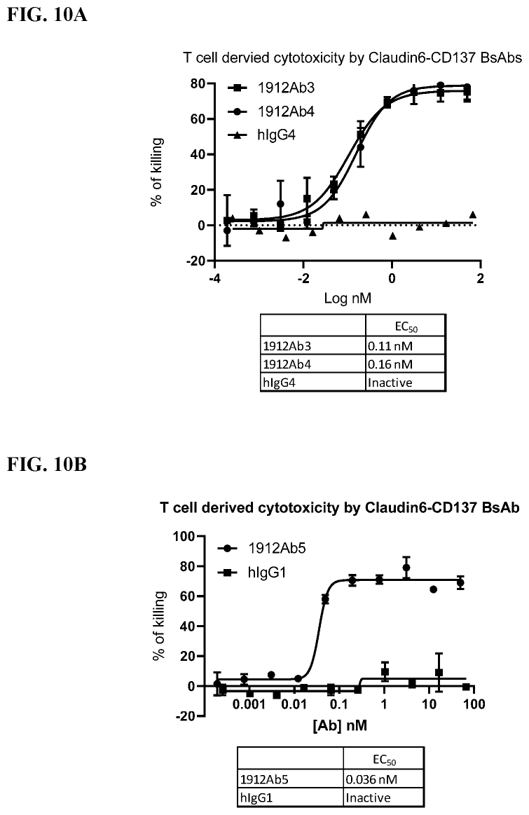

FIG. 10A demonstrates that 1912Ab3 and 1912Ab4 induced strong T cell-mediated cytotoxicity. Around 80% of tumor cells were killed by the CD8 T cells upon 108 hours of treatment with the BsAbs. The EC₅₀ values of 1912Ab3 and 1912Ab4 were 0.11 nM and 0.16 nM, respectively.

To evaluate the TDCC effect of BsAb 1912Ab5, a similar co-culture experiment was performed. In brief, CD8 T cells from a healthy donor were co-cultured with ovarian cancer cell line OV90 cells stably transfected with GFP in the presence of mouse anti-hCD3 clone OKT3 (Biolegend, Cat #: 317325). The co-cultured cells were treated with BsAb 1912Ab5 or control for 144 hours. The disclosed antibody 1912Ab5 is a bispecific antibody that binds to both Claudin 6 and CD137. The number of live cells was measured using Cytation (Biotek, VT). The percentage of killing was calculated by the following formula:

% of killing=(area of GFP cells from well without binding protein treatment-area of GFP cells from well treated with binding protein)/area of GFP cells from well without binding protein treatment*100%

FIG. 10B demonstrates that 1912Ab5 induced strong T cell-mediated cytotoxicity. Around 70% of tumor cells were killed by the CD8 T cells upon 144 hours of treatment with the BsAb. The EC₅₀ value of 1912Ab5 was 0.036 nM.

### Example 10: Effect of CLDN6/CD137 BsAbs on Tumor Growth in a Subcutaneous, Syngeneic MC38-hClaudin 6 Mouse Tumor Model in Humanized B-h4-1BB Mice

Female B-h4-1BB mice (Biocytogen), 6-8 weeks of age, with a body weight between 16-20 g, were acclimated for 7 days prior to study enrollment. The MC38 murine colon carcinoma cell line was genetically modified to overexpress human Claudin 6. Cells were maintained in vitro as monolayer culture in DMEM supplemented with 10% heat inactivated FBS at 37° C. in an atmosphere of 5%. Cells were harvested and 5×10⁵ cells in 100 μl of PBS were subcutaneously implanted into the right front flank for tumor development. On day 7, tumor-bearing mice were randomly enrolled into 3 study groups with the mean tumor size approximately 100-150 mm³. Each group consisted of 6 mice. Tumor size was measured two times weekly in two dimensions using a caliper, and the volume is expressed in mm³ using the formula: V=0.5 a×b² where a and b are the long and short dimensions of the tumor, respectively. On day 7, 11, 14 and 18, mice were treated with an intraperitoneal injection of 5 mg/kg of 1912Ab3, 1912Ab4 or PBS as a negative control. The study was terminated on day 28.

FIG. 11A shows the tumor growth curves for three treatment groups. Both 1912Ab3 and 1912Ab4 significantly inhibited tumor growth compared to vehicle control. All mice injected with 1912Ab3, and 5 out of 6 mice injected with 1912Ab4 showed complete tumor remission on day 28.

Live toxicity has been monitored by mearing the ALT and AST activity in mouse serum from a day 21 serum sample. As shown in FIG. 11B, the ALT level has no significant increase comparing the treated groups with the control group. Similarly, as shown in FIG. 11C, the AST level has no significant increase from the treated groups, indicating a low risk of antibody-derived hepatotoxicity.

To evaluate if bsAbs Claudin6/CD137 can induce tumor immunity in the mice that had complete tumor remission. Four mice were previously treated by 1912Ab3, and four mice treated by 1912Ab were re-challenged by MC38-Claudin6 tumor again 45 days after the last dose of the previous treatment. Four naïve mice were used in this study as the control group. As shown in FIG. 11D, all of the naïve mice developed tumors; however, none of the previously treated mice with complete tumor remission developed a tumor after the rechallenge.

A dose titration study was conducted to explore the efficacious dose of antibody 1912Ab5. Female B-h4-1BB mice from Biocytogen (Boston, MA) were inoculated with 5×10⁵ viable MC38 cells subcutaneously. When the tumor size reached approximately 100 mm³, the mice were randomized into 3 groups, and treatment by intraperitoneal injection was initiated. Group 1 received vehicle control; group 2 received 0.3 mpk 1912Ab5 antibody; group 3 received 1 mpk 1912Ab5 antibody; and group 4 received 3 mpk 1912Ab5 antibody. Treatment was administered twice a week for 2 weeks.

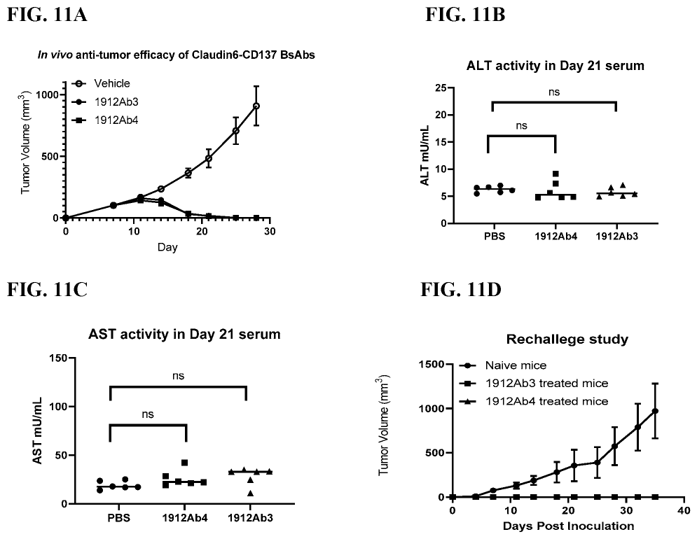

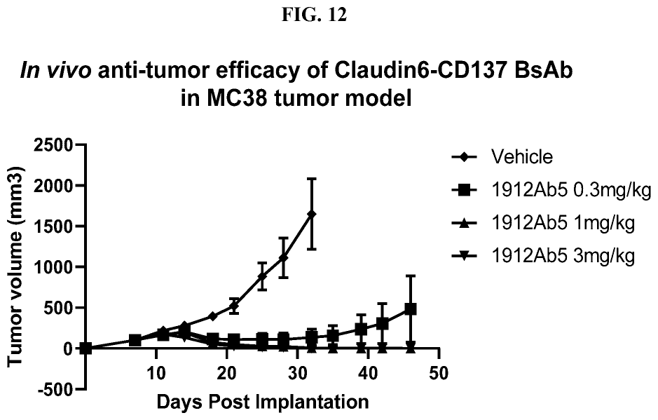

As shown in FIG. 12, single-agent 1912Ab5 demonstrated potent efficacy. At the dose of 0.3 mpk exhibited 97.7% tumor growth inhibition (TGI) on day 32 post tumor inoculation; 1912Ab5 at 1 mpk exhibited 106.2% tumor growth inhibition (TGI) on day 32 post tumor inoculation, and 1912Ab5 at 3 mpk exhibited 106.4% tumor growth inhibition (TGI) on day 32 post tumor inoculation;

A follow-up study was conducted to compare the potency of Ab 1912Ab5 with a benchmark CD137 Ab Urelumab-NR. Female B-h4-1BB mice from Biocytogen (Boston, MA) were inoculated with 5×10⁵ viable MC38 cells subcutaneously. When the tumor size reached approximately 100 mm³, the mice were randomized into 3 groups, and treatment by intraperitoneal injection was initiated. Group 1 received vehicle control; group 2 received 0.1 mpk 1912Ab5 antibody, and group 3 received 0.1 mpk Urelumab-NR. Treatment was administered twice a week for 2 weeks.

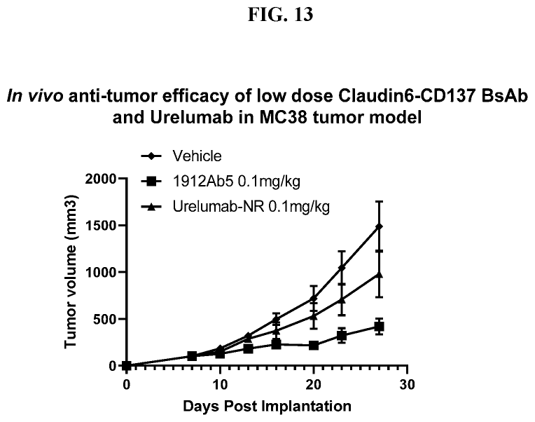

As shown in FIG. 13, single agent 1912Ab5 demonstrated superior efficacy compared to the benchmark antibody Urelumab-NR. At the dose of 0.1 mpk, 1912Ab5 exhibited 77.2% tumor growth inhibition (TGI) on day 27 post tumor inoculation; while Urelumab-NR at 0.1 mpk only exhibited 36.6% tumor growth inhibition (TGI).

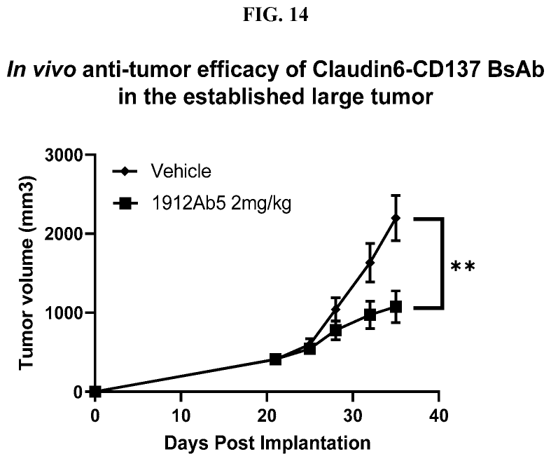

To explore whether Claudin6/CD137 bispecific Ab can be used for treating established large tumors. Female B-h4-1BB mice from Biocytogen (Boston, MA) were inoculated with 5×10⁵ viable MC38 cells subcutaneously. When the tumor size reached approximately 400 mm³, the mice were randomized into 2 groups and treated twice a week for 1 week. Group 1 received vehicle control; group 2 received two doses of 2 mpk 1912Ab5 antibody. As shown in FIG. 14, 1912Ab5 demonstrated potent efficacy-62.7% tumor growth inhibition (TGI) on day 35 post tumor inoculation.

### Example 11: Immune Contexture Analysis of Claudin6-CD137 Antibody-Treated Tumors

A multiplex fluorescent immunohistochemistry (IHC) study and a tumor infiltrated lymphocyte (TIL) analysis were conducted to evaluate the immune cell content in the tumors after Claudin6/CD137 bsAb treatment.

Female B-h4-1BB mice from Biocytogen (Boston, MA) were inoculated with 5×105 viable MC38 cells subcutaneously. When the tumor size reached approximately 100 mm3, the mice were randomized into 2 groups, 8 mice per group, treated twice on day 15 and day 19. Group 1 was treated by vehicle control, and group 2 was treated by 1 mpk 1912Ab5. On day 26 post tumor inoculation, the mice were euthanized and fresh tumors were taken for IHC and Til studies.

Two tumors from each treatment group were formalin-fixed and paraffin-embedded.

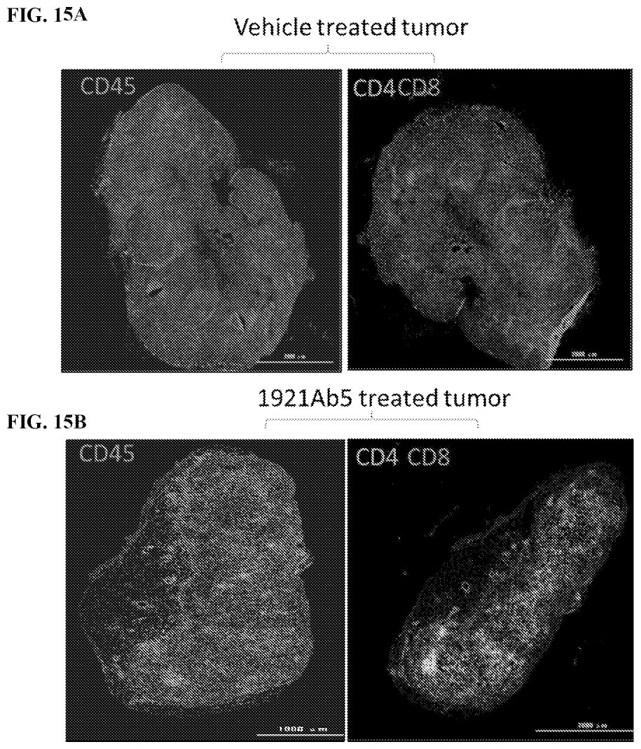

Fluorescent IHC was conducted with 5 mm of FFPE tissue sections. Following deparaffinization, slides were stained by primary antibodies detecting CD45, CD3, CD4 and CD8 for multiplexed immune cell profiling. Representative images are shown in FIG. 15. The 1912Ab5 treated tumors had significantly increased lymphocyte infiltration, and CD4 and CD8 T cell infiltration (FIG. 15B) as compared to vehicle control (FIG. 15A).

A tumor infiltrated lymphocyte analysis was performed using 6 fresh tumors from each treatment group. The enzymatic-based method was used for dissociating tumors. The cells from the digested tumors were filtered, washed, and used for the multiplexed flow cytometry. T cell population was gated by live CD45+CD3+; CD4T cells were gated by live CD45+CD3+CD4+CD8−; CD8 T cells were gated by live CD45+CD3+CD4-CD8+; exhausted T cells were gated by live CD45+CD3+ Tim-3+; central memory T cells were gated by CD45+CD3+CD8+CD44high CD62Lhigh; resident memory T cells were gated by live CD45+CD3+CD8+CD69+CD103+; and M2-like macrophage cells were gated by live CD45+CD11b+F4/80++CD206+.

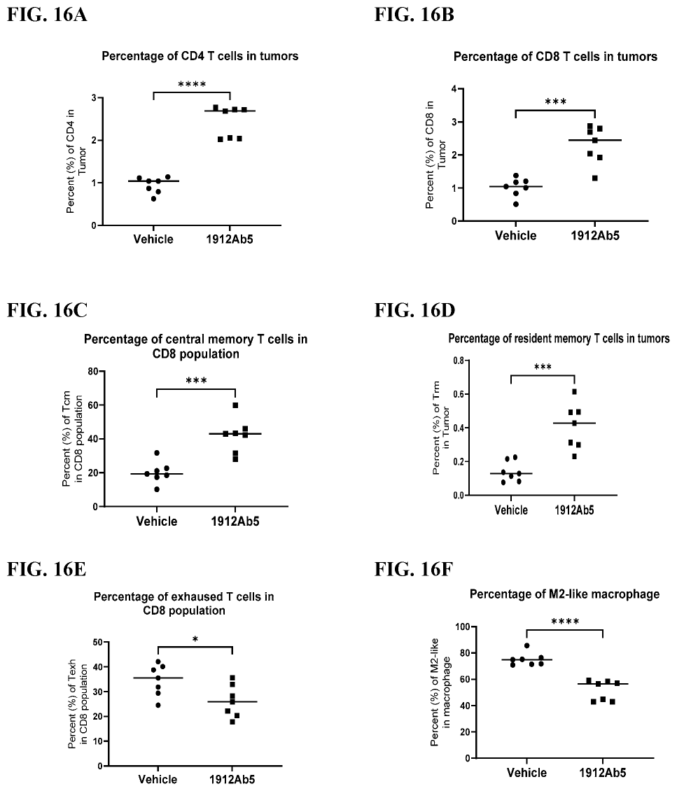

As shown in FIG. 16, in all 1912Ab5 treated tumors, both of the CD4 cells (FIG. 16A) and CD8 cells (FIG. 16B) increased, suggesting an enhanced T-cell derived tumor-killing. Moreover, the T memory cells, central memory T cells (FIG. 16C) and resident memory T cells (FIG. 16D) increased significantly, together with the tumor immunity observed in 1912Ab5 treated animals (FIG. 11D) suggesting the Claudin6-CD137 bsAb treatment may promote anti-tumor memory formation. Additionally, the exhaust T cell decrease (FIG. 16E) M2-like macrophage reduction (FIG. 16F) indicates the tumor microenvironment modulating effect of the Claudin6-CD137 bsAb.

Based on the data disclosed herein, it is anticipated that the anti-tumor effect of CLDN6/CD137 BsAb treatment will result in a modulated TME, the increased lymphocyte infiltration will turn a suppressive TME into an inflammatory TME. The tumor cell-dependent CD137 activation would specifically enhance the tumor experienced T cell activation and promote T memory formation, the fast on fast off kinetics of the bsAb can help reduce T cell exhaustion. The reduced M2 macrophages will reduce suppressive cytokine release in TME hence improving T cell activation.

### Example 12: Evaluation of a Claudin6-CD137 Antibody in a PD1 Resistant Model B16F10

To predict the therapeutic potential of Claudin6/CD137 antibody treatment in PD1 resistant patients, a PD1 resistant tumor model B16F10 melanoma model was used to evaluate the efficacy of antibody 1912Ab5. Six to seven-week-old female homozygous B-h4-1BB mice from Biocytogen (Boston, MA) were injected with 1×10⁵ viable B16-F10 cells subcutaneously into the right flank. When the tumor size reached between 75 and 100 mm³, the mice were randomized into two groups, and treatment by intraperitoneal injection was initiated. Group 1 received vehicle control; group 2 received 3 mpk 1912Ab5 antibody. Treatment was administered twice a week for 2 weeks.

Body weights were measured twice weekly. Tumor volumes were determined at different time points using the formula V=½*L×W×W, where L is the long dimension and W is the short dimension of the xenograft. Any mice with tumors over 2500 mm³ were sacrificed. The survival of the mice was monitored up to 27 days post tumor implantation.

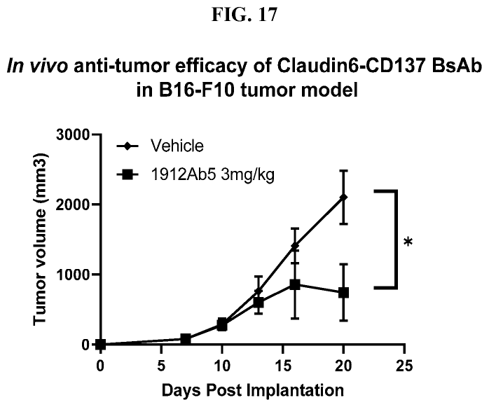

As shown in FIG. 17, mice from the 3 mpk 1912Ab5 antibody treated group had a TGI value of 67.1% on day 20 post tumor implantation and demonstrated significant efficacy.

### Example 13: Binding of CLDN18.2/CD137 BsAbs to Claudin 18.2 on the Cell Surface

Bispecific CLDN18.2/CD137 antibodies were generated, produced, and purified as described in Example 3. To examine the binding activity of these binding proteins to Claudin 18.2, NUGC4 cells endogenously expressing human Claudin 18.2 were used in an immunofluorescence binding assay. The cells were cultured in RPMI media with 10% FBS. On the experiment day, the cells were collected, washed, and stained with the BsAbs 1901Ab2 and 1901Ab3, and monospecific anti-CLDN18.2 control antibody 1901Ab1 at 4° C. for 2 hours, followed by fixing cells with paraformaldehyde for 15 minutes at room temperature. The monoclonal antibody 1901Ab1 specifically binds to Claudin 18.2, The fixed cells were then washed with PBS three times following by staining the cells at room temperature for 1 hour with Alexa Fluor® 488 Goat Anti-Human IgG antibody (Invitrogen, Cat #: A-11013) for detection. The binding signal was assessed by quantifying the fluorescence intensity using iQue Screener PLUS (Sartorius, MI).

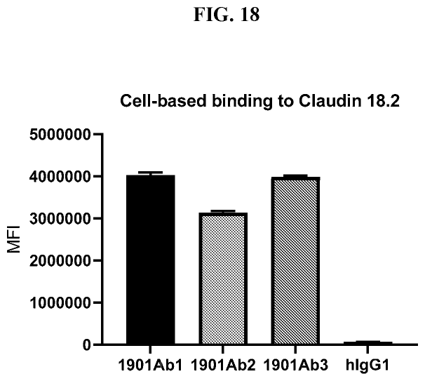

As shown in FIG. 18, at the concentration of 10 μg/ml, the disclosed bispecific binding proteins including 1901Ab2 and 1901Ab3 bound similarly to human Claudin 18.2 on NUGC4 cells compared to the control monoclonal antibody 1901Ab1.

### Example 14: Binding of CLDN18/CD137 BsAbs to CD137 on the Cell Surface

The binding affinity of the CLDN18.2/CD137 BsAbs 1901Ab2 and 1901Ab3 was evaluated using an immunofluorescence imaging assay. Briefly, HEK293T-huCD137 cells stably expressing human CD137 were plated in complete media containing DMEM with 10% FBS, then incubated overnight at 37° C. Cells were bound with the BsAbs at 4° C. for 2 hours, followed by fixing cells with paraformaldehyde for 15 minutes at room temperature. Monospecific antibody 1923 Ab4 only binds to CD137 and is used as a control antibody. The fixed cells were washed with PBS three times followed by staining at room temperature for 1 hour with Alexa Fluor® 488 Goat Anti-Human IgG antibody (Invitrogen, Cat #: A-11013) for detection. The binding signal was assessed by imaging the cells and quantifying the fluorescence intensity using Cytation Imager (Biotek, VT).

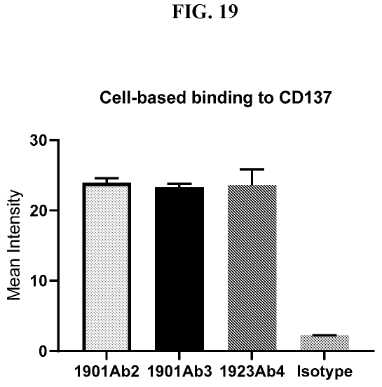

As demonstrated in FIG. 19, at the concentration of 10 μg/ml, the disclosed bispecific binding proteins 1901Ab2 and 1901Ab3 bound similarly to human CD137 on the cell surface as their monoclonal antibody control 1923Ab4.

### Example 15: Target Cell-Dependent Activation of CD137 Signaling by CLDN18.2/CD137 BsAbs

The anti-CLDN18.2/CD137 BsAbs 1901Ab2 and 1901Ab3 were also evaluated for their ability to induce target cell-dependent CD137 agonism. In brief, Jurkat T reporter cell line stably expressing CD137 and containing NFKB-luc report was used to quantify CD137 signaling. NUGC4 cells, which expressed endogenous Claudin 18.2 on the cell surface, were used as target cells. The Jurkat T reporter cells were co-cultured with or without NUGC4 target cells and were stimulated with the disclosed binding proteins for 16 hours at 37° C. with 5% CO₂. ONE-Glo™ luciferase reagent (Promega, Cat #: E6130) was then added, and the plate was incubated at room temperature for 10 minutes. Monospecific antibody Urelumab-NR (generated by NovaRock Biotherapeutics based on publicly available sequence information) only binds to CD137 and is used as a control antibody. The luminescence signal was measured by Synergy Neo2 plate reader (Biotek) and data was analyzed by GraphPad Prism.

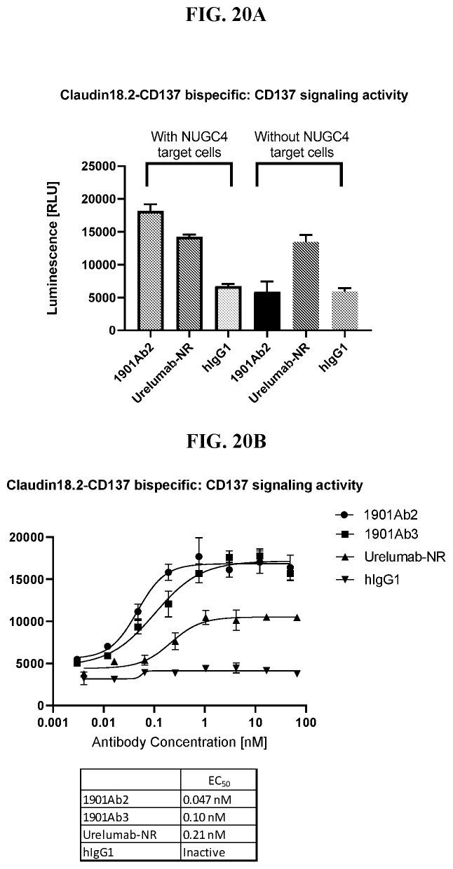

FIG. 20A demonstrates that, as expected, Urelumab-NR activated CD137 signaling independent of the presence of NUGC4 target cells. In contrast, 1901Ab2 induced CD137 signaling only in the presence of NUGC4 target cells. Furthermore, 1901 Ab2 induced more robust CD137 signaling than Urelumab-NR in the presence of NUGC4 target cells. This result confirms that 1901Ab2 only showed CD137 agonism when the binding protein engaged Claudin 18.2 expressing on the cell surface of NUGC4 cells. No agonistic activity of 1901Ab2 was detected in the absence of NUGC4 cells.

The dose-response curves of 1901Ab2, 1901Ab3 and Urelumab-NR to induce CD137 signaling in the presence of NUGC4 cells were shown in FIG. 20B. The EC₅₀ values (potency) of 1901Ab2, 1901Ab3 and Urelumab-NR were 0.047 nM, 0.10 nM and 0.21 nM, respectively. Both 1901Ab2 and 1901 Ab3 demonstrated better EC₅₀ value and higher signaling strength (Eₘₐₓ) than Urelumab-NR to induce CD137 signaling.

### Example 16: Activation of CD8 T Cells by CLDN18.2/CD137 BsAbs

A co-culture experiment was used to measure T cell activation by the BsAbs 1901Ab2 and 1901Ab3 in the presence of TCR signaling. CD8 T cells from a healthy donor and NUGC4 cells were used as effector and target cells. These two cells were cultured together in RPMI1640 media supplemented with 10% FBS and 0.5 ug/ml of mouse anti-hCD3 clone OKT3 (Biolegend, Cat #: 317325) provides TCR signaling. The disclosed binding proteins were added to stimulate T cells. The plate was incubated for 3 days at 37° C. with 5% CO₂. After 72 hours of incubation, supernatants were collected and used to measure the secreted IFNγ by AlphaLISA (PerkinElmer, Cat #: AL217C/F) using protocols according to the manufacturer's instruction. The amount of IFNγ represents T cell activation.

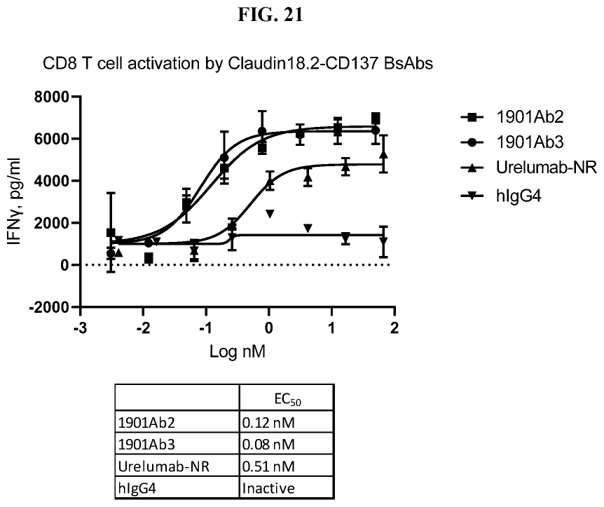

The dose-response curves of 1901Ab2, 1901Ab3 and Urelumab-NR to induce CD8 T cell activation in the presence of NUGC4 cells are shown in FIG. 21. The EC₅₀ values of 1901Ab2, 1901Ab3 and Urelumab-NR were 0.081 nM, 0.12 nM and 0.51 nM, respectively. Both 1901Ab2 and 1901Ab3 demonstrated better potency and higher Emax than Urelumab-NR to induce IFNγ production, which is a hallmark of T cell activation.

### Example 17: CD8 T Cell Derived Tumor Killing Activity Induced by CLDN18.2/CD137 BsAbs

A co-culture experiment was performed to measure the tumor-killing activity of CD8 T cells treated with the CLDN18.2/CD137 BsAbs 1901Ab2 and 1901Ab3. In brief, CD8 T cells from a healthy donor were pre-activated with ImmunoCult™ Human CD3/CD28 T Cell Activator (Stemcell, Cat #: 10971) for 2 days. Next, the activated cells were washed to removed CD3/CD28 activator. The activated CD8 T cells were then co-cultured with NUGC4 tumor cells stably transfected with GFP and treated with the disclosed bispecific binding proteins for 96 hours. The number of cells was measured by green fluorescent intensity using Cytation (Biotek, VT). The percentage of killing was calculated by the following formula:

% of killing=(GFP signal from well without binding protein treatment-GFP signal from well treated with antibody)/GFP signal from well without binding protein treatment*100%

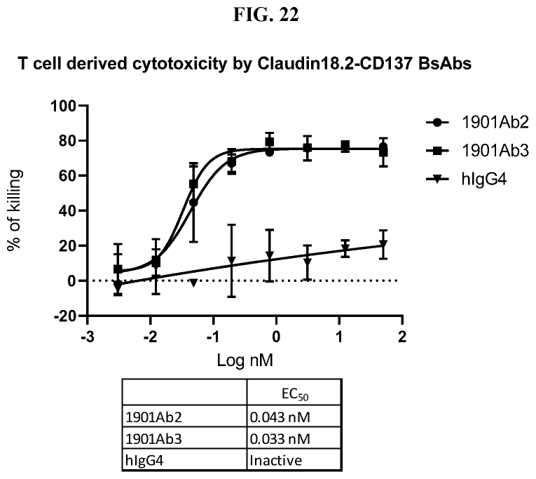

FIG. 22 demonstrates that 1901Ab2 and 1901Ab3 induced strong T cell-mediated cytotoxicity. Around 75% of tumor cells were killed by CD8 T cells upon 96 hours of treatment with the disclosed bispecific binding proteins. The EC₅₀ values of 1901Ab2 and 1901Ab3 are 0.043 nM and 0.033 nM, respectively.

### Example 18: Effect of CLDN18.2/CD137 BsAb 1901Ab2 on Tumor Growth in a Subcutaneous, Syngeneic MC38-hClaudin 18.2 Mouse Tumor Model in Humanized B-h4-1BB Mice

Female B-h4-1BB mice (Biocytogen), 6-8 weeks of age, with bodyweight between 16-20 g, were acclimated for 7 days prior to study enrollment. The MC38 murine colon carcinoma cell line was genetically modified to overexpress human Claudin 18.2. Cells are maintained in vitro as a monolayer culture in DMEM supplemented with 10% heat-inactivated FBS at 37° C. in an atmosphere of 5%. Cells were harvested and 5×10⁵ cells in 100 μl of PBS were subcutaneously implanted into the right front flank for tumor development. On day 7, tumor-bearing mice were randomly enrolled into 3 study groups (each group comprising 6 mice) with the mean tumor size approximately 100-150 mm³. Tumor size was measured two times weekly in two dimensions using a caliper, and the volume is expressed in mm³ using the formula: V=0.5 a×b² where a and b are the long and short dimensions of the tumor, respectively. On days 7, 10, 14, and 17, mice were treated by 5 mg/kg 1901Ab2 or PBS control by intraperitoneal injection. The study was terminated on day 34.

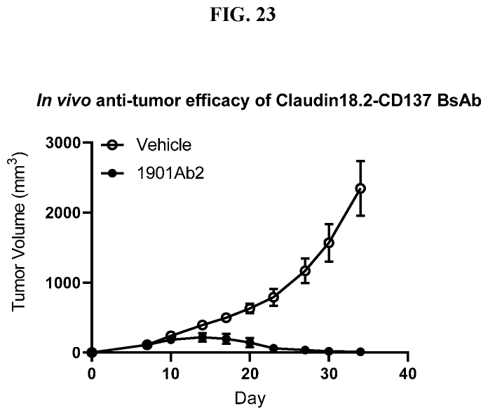

FIG. 23 shows the tumor growth curves for two treatment groups. 1901Ab2 significantly inhibited tumor growth compared to PBS treatment control. 4 out of 6 mice injected with 1901Ab2 showed complete tumor remission on day 34.

### Example 19: Binding of Nectin-4/CD137 BsAbs to Nectin 4 on the Cell Surface

Bispecific Nectin-4/CD137 binding proteins were generated, produced, and purified as described in Example 4. To examine the binding activity of the BsAbs 1925Ab1, 1925Ab2 and 1925Ab3 to Nectin 4, CHO cells expressing human Nectin 4 were used in an immunofluorescence binding assay. The cells were cultured in F12K media with 10% FBS. On the day of the experiment, the cells were collected, washed, and stained with the binding proteins at 4° C. for 2 hours, followed by fixing cells with paraformaldehyde for 15 minutes at room temperature. The disclosed antibodies 1925Ab1, 1925Ab2 and 1925Ab3 are bispecific antibodies that bind to both Nectin-4 and CD137. Monospecific antibody 1925Ab4 (parental murine antibody) only binds to Nectin-4 and is used as a control antibody. The fixed cells were then washed with PBS three times, followed by staining the cells at room temperature for 1 hour with Alexa Fluor® 488 Goat Anti-Human IgG antibody (Invitrogen, Cat #: A-11013) for detection. The binding signal was assessed by quantifying the fluorescence intensity using iQue Screener PLUS (Sartorius, MI).

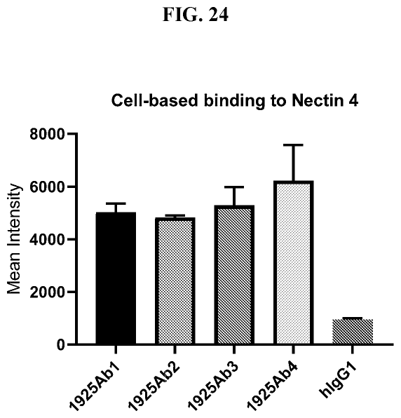

As shown in FIG. 24, at the concentration of 10 μg/ml, the disclosed bispecific binding proteins, including 1925Ab1, 1925Ab2 and 1925Ab3 bound similarly to human Nectin 4 on CHO cells compared to the control monoclonal antibody 1925Ab4.

### Example 20: Binding of Nectin-4/CD137 BsAbs to CD137 on the Cell Surface

The binding affinity of BsAbs 1925Ab1, 1925Ab2 and 1925Ab3 was evaluated using an immunofluorescence imaging assay. The HEK293T-huCD137 cells stably expressing human CD137 were plated in complete media containing DMEM with 10% FBS, then incubated overnight at 37° C. Cells were bound with the disclosed binding proteins at 4° C. for 2 hours followed by fixing cells with paraformaldehyde for 15 minutes at room temperature. The disclosed antibodies 1925Ab1, 1925Ab2 and 1925Ab3 are bispecific antibodies that bind to both Nectin-4 and CD137. Monospecific anti-CD137 antibody 1923Ab4 only binds to CD137 and is used as a control antibody. The fixed cells were washed with PBS three times, followed by staining at room temperature for 1 hour with Alexa Fluor® 488 Goat Anti-Human IgG antibody (Invitrogen, Cat #: A-11013) for detection. The binding signal was assessed by imaging the cells and quantifying the fluorescence intensity using Cytation Imager (Biotek, VT).

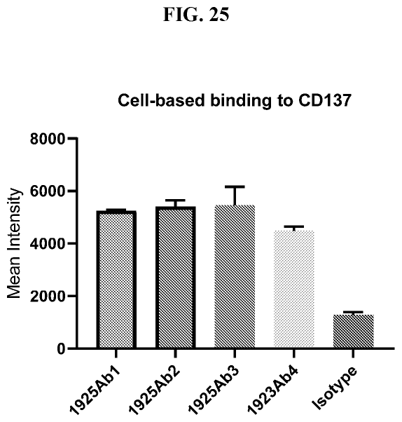

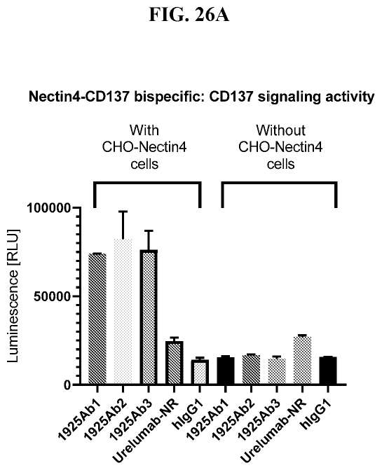

As demonstrated in FIG. 25, at the concentration of 10 μg/ml, the disclosed bispecific binding proteins including 1925Ab1, 1925Ab2 and 1925Ab3 bound similarly to human CD137 on the cell surface as their monoclonal antibody control 1923Ab4.

### Example 21: Target Cell-Dependent Activation of CD137 Signaling by Nectin-4/CD137 BsAbs

The Nectin-4/CD137 BsAbs 1925Ab1, 1925Ab2, 1925Ab3 were evaluated for their ability to induce target cell-dependent CD137 agonism. In brief, Jurkat T reporter cell line stably expressing CD137 and containing NFKB-luc report was used to quantify CD137 signaling. CHO cells stably transfected with human Nectin 4 (CHO-Nectin 4) were used as target cells. The Jurkat T reporter cells were co-cultured with or without CHO-Nectin 4 target cells and were stimulated with the disclosed binding proteins for 16 hours at 37° C. with 5% CO₂. ONE-Glo™ luciferase reagent (Promega, Cat #: E6130) was then added, and the plate was incubated at room temperature for 10 minutes. The disclosed antibodies 1925Ab1, 1925Ab2 and 1925Ab3 are bispecific antibodies that bind to both Nectin-4 and CD137. Monospecific antibody Urelumab-NR only binds to CD137 and is used as a control antibody. The luminescence signal was measured by Synergy Neo2 plate reader (Biotek) and data was analyzed by GraphPad Prism.

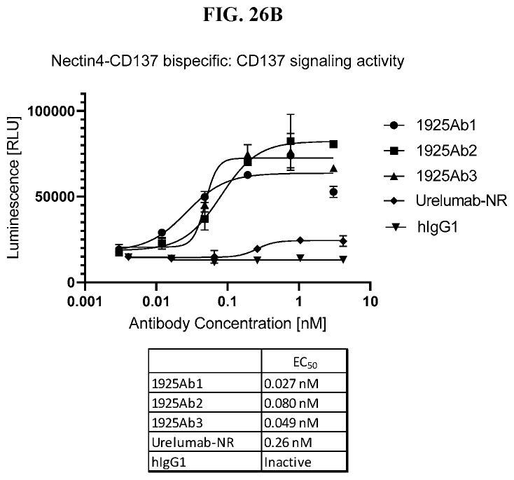

Urelumab-NR was generated by NovaRock Biotherapeutics based on the publicly available sequence of Urelumab. FIG. 26A demonstrates that, as expected, Urelumab-NR activated CD137 signaling independent of the presence of CHO-Nectin 4 target cells. In contrast, 1925Ab1, 1925Ab2 and 1925Ab3 induced CD137 signaling only in the presence of CHO-Nectin 4 target cells. Furthermore, 1925Ab1, 1925Ab2 and 1925Ab3 induced more robust CD137 signaling than Urelumab-NR in the presence of NUGC4 target cells. This result confirms that the disclosed bispecific binding proteins only showed CD137 agonism when the binding proteins engaged Nectin 4 expressing on the cell surface of CHO cells. No agonistic activity of these disclosed bispecific binding proteins was detected in the absence of CHO-Nectin 4 cells.

The dose-response curves of 1925Ab1, 1925Ab2, 1925Ab3 and Urelumab-NR to induce CD137 signaling in the presence of NUGC4 cells were shown in FIG. 26B. The EC₅₀ values (potency) of 1925Ab1, 1925Ab2, 1925Ab3 and Urelumab-NR were 0.027 nM, 0.080 nM, 0.049 nM and 0.26 nM, respectively. The disclosed bispecific binding proteins including 1925Ab1, 1925Ab2 and 1925Ab3 demonstrated better EC₅₀ value and higher signaling strength (Eₘₐₓ) than Urelumab-NR to induce CD137 signaling.

### Example 22: Evaluation of the Antibody-Derived Immune Cell Infiltration in Mouse Liver

Liver toxicity has been a know side effect of some of the earlier CD137 agonist antibody therapeutics. Urelumab was reported to induce inflammatory hepatotoxicity at doses≥0.3 mg/kg, with a maximum tolerated dose (MTD) of 0.1 mg/kg, limiting its therapeutic window (Segal N H, et al. Clin Cancer Res. 2017; 23 (8): 1929-1936). Studies have shown that liver toxicity by Urelumab is from liver inflammation, indicated by immune cell infiltration in the liver and significantly elevated serum ALT levels (Zhang H, et al. Journal for ImmunoTherapy of Cancer 2020; 8).

Bispecific antibodies that bind to CD137 and a tumor associated antigen (TAA) offer the advantages of potent co-stimulation targeted to the tumor microenvironment (TME) and a diminished risk of liver inflammation/hepatotoxicity, which can broaden the therapeutic window.

To demonstrate the lower liver toxicity of the disclosed bispecific TAA-CD137 antibodies, female B-h4-1BB mice (Biocytogen), 6-8 weeks of age, with a body weight between 16-20 g, were acclimated for 7 days prior to study enrollment. B-h4-1BB mice were randomly enrolled into three study groups. Each group consisted of 6 mice. On day 0, 3, 7 and 10, mice were treated with an intraperitoneal injection of 10 mg/kg of Urelumab-NR, 1912Ab5 or PBS as a negative control. The study was terminated on day 13. The livers from each treatment group were formalin-fixed and paraffin-embedded. Fluorescent IHC was conducted with 5 mm of FFPE tissue sections. Following deparaffinization, slides were stained by primary antibodies detecting CD4, CD8 T cells and macrophages for immune cell identification.

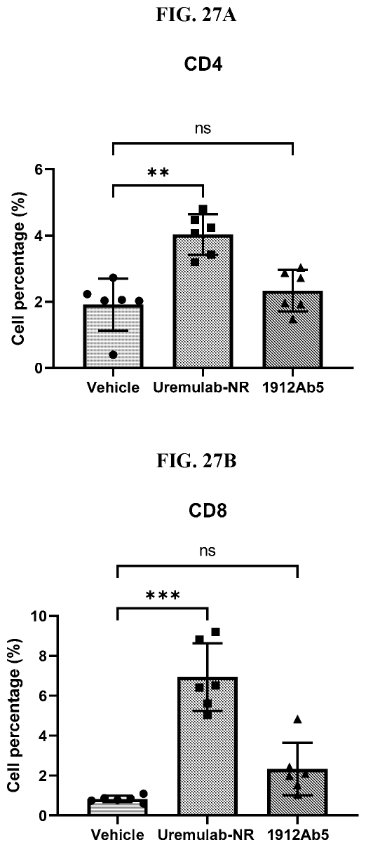

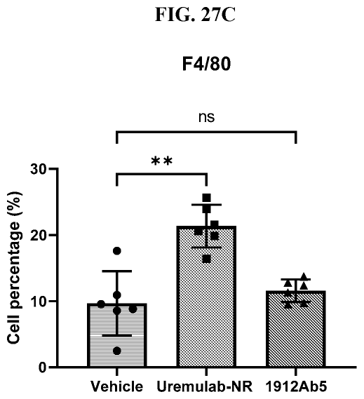

In FIG. 27, mouse CD4 (A), CD8 (B) T cell, and mouse macrophages (C) infiltration increased significantly only in the Urelumab-NR treated mice but not in the 1912Ab5 treated mice.

Consistent with Example 10, FIGS. 11B and 11C, the lack of immune cell infiltration induced by 1912Ab5 indicates a low risk of CLDN6/CD137 bsAb-derived hepatotoxicity.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A bispecific binding protein that binds a tumor associated antigen and CD137 comprising:
(a) an antibody scaffold module comprising a first antigen-binding site that binds the tumor associated antigen and a second antigen-binding site that binds the tumor associated antigen;
(b) at least one first binding module comprising a third antigen-binding site that binds CD137;
wherein the tumor associate antigen is Claudin 6, Claudin 18.2, or Nectin 4.

2.-6. (canceled)

7. The bispecific binding protein of claim 1, wherein the first antigen-binding site and the second antigen-binding site both bind Claudin 6 and comprise:
(i) a heavy chain variable region sequence comprising CDR1: SEQ ID NO: 45, CDR2: SEQ ID NO: 46, and CDR3: SEQ ID NO: 47; and a light chain variable region sequence comprising CDR1: SEQ ID NO: 48, CDR2: SEQ ID NO: 49, and CDR3: SEQ ID NO: 50; or
(ii) a heavy chain variable region sequence comprising CDR1: SEQ ID NO: 51, CDR2: SEQ ID NO: 52, and CDR3: SEQ ID NO: 53; and a light chain variable region sequence comprising CDR1: SEQ ID NO: 54, CDR2: SEQ ID NO: 55, and CDR3: SEQ ID NO: 56.

8. (canceled)

9. The bispecific binding protein of claim 7, wherein the first antigen-binding site and the second antigen-binding site both bind Claudin 6 and comprise:
(i) a heavy chain variable region sequence as set forth in SEQ ID NO: 25 and a light chain variable region sequence as set forth in SEQ ID NO: 26; or
(ii) a heavy chain variable region sequence as set forth in SEQ ID NO: 27 and a light chain variable region sequence as set forth in SEQ ID NO: 28.

10. The bispecific binding protein of claim 1, wherein the first antigen-binding site and the second antigen-binding site both bind Claudin 18.2 and comprise:
a heavy chain variable region sequence comprising CDR1: SEQ ID NO: 33, CDR2: SEQ ID NO: 34, and CDR3: SEQ ID NO: 35; and a light chain variable region sequence comprising CDR1: SEQ ID NO: 36, CDR2: SEQ ID NO: 37, and CDR3: SEQ ID NO: 38.

11. The bispecific binding protein of claim 10, wherein the first antigen-binding site and the second antigen-binding site both bind Claudin 18.2 and comprise a heavy chain variable region sequence as set forth in SEQ ID NO: 21; and a light chain variable region sequence as set forth in SEQ ID NO: 22.

12. The bispecific binding protein of claim 1, wherein the first antigen-binding site and the second antigen-binding site both bind Nectin-4 and comprise:
a heavy chain variable region sequence comprising CDR1: SEQ ID NO: 57, CDR2: SEQ ID NO: 58, and CDR3: SEQ ID NO: 59; and a light chain variable region sequence comprising CDR1: SEQ ID NO: 60, CDR2: SEQ ID NO: 61, and CDR3: SEQ ID NO: 62.

13. (canceled)

14. The bispecific binding protein of claim 12, wherein the first antigen-binding site and the second antigen-binding site both bind Nectin-4 and comprise:
(i) a heavy chain variable region sequence as set forth in SEQ ID NO: 29 and a light chain variable region sequence as set forth in SEQ ID NO: 30; or
(ii) a heavy chain variable region sequence as set forth in SEQ ID NO: 31 and a light chain variable region sequence as set forth in SEQ ID NO: 32.

15. (canceled)

16. (canceled)

17. The bispecific binding protein of claim 1, wherein the first binding module is an antibody fragment.

18. (canceled)

19. (canceled)

20. The bispecific binding protein of claim 1, wherein the first binding module is an scFV that binds CD137.

21. (canceled)

22. The bispecific binding protein of claim 1, wherein the first binding module and the antibody scaffold module are covalently attached to each other through an interlinker having a sequence as set forth in SEQ ID NO: 64 or SEQ ID NO: 65.

23.-25. (canceled)

26. The bispecific binding protein of claim 1, wherein the first binding module binds CD137 and comprises:
a heavy chain variable region sequence comprising CDR1: SEQ ID NO: 39, CDR2: SEQ ID NO: 40, and CDR3: SEQ ID NO: 41; and a light chain variable region sequence comprising CDR1: SEQ ID NO: 42, CDR2: SEQ ID NO: 43, and CDR3: SEQ ID NO: 44.

27. The bispecific binding protein of claim 26, wherein the first binding module bind CD137 and comprises: a heavy chain variable region sequence as set forth in SEQ ID NO: 23; and a light chain variable region sequence as set forth in SEQ ID NO: 24.

28. The bispecific binding protein of claim 1, wherein the bispecific binding protein comprises two first binding modules that bind CD137 and wherein:
the first antigen-binding site and the second antigen-binding site both bind Claudin 18.2 and comprise a heavy chain variable region sequence as set forth in SEQ ID NO: 21, and a light chain variable region sequence as set forth in SEQ ID NO: 22;
the first binding modules each comprise a heavy chain variable region sequence as set forth in SEQ ID NO: 23, and a light chain variable region sequence as set forth in SEQ ID NO: 24,
wherein the first binding modules are separately attached to the C-terminus of each heavy chain in the antibody scaffold module by a glycine-serine linker.

29.-32. (canceled)

33. The bispecific binding protein of claim 1, wherein the bispecific binding protein comprises two first binding modules that bind CD137 and wherein:
the first antigen-binding site and the second antigen-binding site both bind Claudin 18.2 and comprise a heavy chain variable region sequence as set forth in SEQ ID NO: 21, and a light chain variable region sequence as set forth in SEQ ID NO: 22;
the first binding modules each comprise a heavy chain variable region sequence as set forth in SEQ ID NO: 23, and a light chain variable region sequence as set forth in SEQ ID NO: 24,
wherein the first binding modules are separately attached to the C-terminus of each light chain in the antibody scaffold module by a glycine-serine linker.

34.-37. (canceled)

38. The bispecific binding protein of claim 1, wherein the bispecific binding protein comprises two first binding modules that bind CD137 and wherein:
the first antigen-binding site and the second antigen-binding site both bind Claudin 6 and comprise a heavy chain variable region sequence as set forth in SEQ ID NOs: 25 or 27, and a light chain variable region sequence as set forth in SEQ ID NOs: 26 or 28;
the first binding modules each comprise a heavy chain variable region sequence as set forth in SEQ ID NO: 23, and a light chain variable region sequence as set forth in SEQ ID NO: 24,
wherein the first binding modules are separately attached to the C-terminus of each heavy chain in the antibody scaffold module by a glycine-serine linker.

39.-42. (canceled)

43. The bispecific binding protein of claim 1, wherein the bispecific binding protein comprises two first binding modules that bind CD137 and wherein:
the first antigen-binding site and the second antigen-binding site both bind Nectin-4 and comprise a heavy chain variable region sequence as set forth in SEQ ID NOs: 29 or 31, and a light chain variable region sequence as set forth in SEQ ID NOs: 30 or 32;
the first binding modules each comprise a heavy chain variable region sequence as set forth in SEQ ID NO: 23, and a light chain variable region sequence as set forth in SEQ ID NO: 24,
wherein the first binding modules are separately attached to the C-terminus of each light chain in the antibody scaffold module by a glycine-serine linker.

44.-47. (canceled)

48. The bispecific binding protein of claim 1, wherein the bispecific binding protein comprises two first binding modules that bind CD137 and wherein:
the first antigen-binding site and the second antigen-binding site both bind Nectin-4 and comprise a heavy chain variable region sequence as set forth in SEQ ID NOs: 29 or 31, and a light chain variable region sequence as set forth in SEQ ID NOs: 30 or 32;
the first binding modules each comprise a heavy chain variable region sequence as set forth in SEQ ID NO: 23, and a light chain variable region sequence as set forth in SEQ ID NO: 24,
wherein the first binding modules are separately attached to the N-terminus of each heavy chain in the antibody scaffold module by a glycine-serine linker.

49-52. (canceled)

53. The bispecific binding protein of claim 1, wherein the bispecific binding protein comprises two first binding modules that bind CD137 and wherein:
the first antigen-binding site and the second antigen-binding site both bind Nectin-4 and comprise a heavy chain variable region sequence as set forth in SEQ ID NOs: 29 or 31, and a light chain variable region sequence as set forth in SEQ ID NOs: 30 or 32;
the first binding modules each comprise a heavy chain variable region sequence as set forth in SEQ ID NO: 23, and a light chain variable region sequence as set forth in SEQ ID NO: 24,
wherein the first binding modules are separately attached to the C-terminus of each heavy chain in the antibody scaffold module by a glycine-serine linker.

54.-57. (canceled)

58. The bispecific binding protein of claim 1, wherein the antibody scaffold module further comprises a constant region.

59. (canceled)

60. (canceled)

61. The binding protein of claim 58, wherein the constant region comprises SEQ ID NO: 66, SEQ ID NO: 67, SEQ ID NO: 68, SEQ ID NO: 69 or SEQ ID NO: 73.

62. A pharmaceutical composition comprising the bispecific binding protein of claim 1 and a pharmaceutically acceptable carrier.

63. A method of treating or preventing cancer, the method comprising administering the bispecific binding protein of claim 1 to a patient in need thereof.

64. A nucleic acid composition comprising one or more nucleic acids encoding the bispecific binding protein of claim 1.

65. An expression vector composition comprising one or more expression vectors comprising the nucleic acid composition of claim 64.

66. A host cell comprising the expression vector composition of claim 65.

67. A method for the production of the bispecific binding protein of claim 1, the method comprising culturing the host cell of claim 66.
